(19) United States
(12) Patent Application Publication
(10) Pub. No.: US 2021/0403954 A1
(43) Pub. Date: Dec. 30, 2021

(54) METHOD OF PRODUCING ISOPRENOID COMPOUND

(71) Applicant: Ajinomoto Co., Inc., Tokyo (JP)

(72) Inventors: Yoshinori TAJIMA, Kawasaki-shi (JP); Hiroaki RACHI, Kawasaki-shi (JP); Yosuke NISHIO, Kawasaki-shi (JP); Zhanna Iosifovna KATASHKINA, Moscow (RU); Valerievich Bylino OLEG, Moscow (RU)

(73) Assignee: Ajinomoto Co., Inc., Tokyo (JP)

(21) Appl. No.: 17/328,608

(22) Filed: May 24, 2021

Related U.S. Application Data
(62) Division of application No. 15/604,772, filed on May 25, 2017, now abandoned.
Application No. 15/604,772 is a continuation of application No. PCT/JP2015/083498, filed on Nov. 27, 2015.

(30) Foreign Application Priority Data
Nov. 28, 2014 (RU) 2014-148144
Sep. 25, 2015 (RU) 2015-141007

(51) Int. Cl. C12P 5/00; C12P 5/02; C08F 36/08

(52) U.S. Cl. CPC C12P 5/007; C08F 36/08; C12P 5/026; C12P 5/00

(57) ABSTRACT

Producing an isoprenoid compound by:
1) culturing an isoprenoid compound-forming microorganism in the presence of a growth promoting agent at a sufficient concentration to grow the isoprenoid compound-forming microorganism;
2) decreasing a concentration of the growth promoting agent to induce formation of the isoprenoid compound by the isoprenoid compound-forming microorganism; and
3) culturing the isoprenoid compound-forming microorganism to form the isoprenoid compound,
is characterized in that the growth phase of the isoprenoid compound-forming microorganism is separated from the formation phase of the isoprenoid compound.

### CROSS REFERENCES TO RELATED APPLICATIONS

This application is a division of U.S. patent application Ser. No. 15/604,772, filed on May 25, 2017, which was a continuation of International Patent Application No. PCT/JP2015/083498, filed on Nov. 27, 2015, and claims priority to Russian Federation Patent Application No. 2014148144, filed on Nov. 28, 2014, and Russian Federation Patent Application No. 2015141007, filed on Sep. 25, 2015, all of which are incorporated herein by reference in their entireties.

### BACKGROUND OF THE INVENTION

### Field of the Invention

The present invention relates to methods for producing an isoprenoid compound such as an isoprene monomer, and the like.

### Discussion of the Background

Natural rubber is a very important raw material in the industries for production of tire and rubber. While its demand will be expanded in future due to motorization mainly in emerging countries, it is not easy to increase agricultural farms in view of regulation for deforestation and competition with palm plantations. Thus, it is difficult to anticipate a yield increase of the natural rubber, and the balance of the demand and supply of the natural rubber is predicted to become tight. Synthesized polyisoprene is available as a material in place of the natural rubber. Its raw material is an isoprene monomer (herein also referred to as “isoprene”). The isoprene (2-methyl-1,3-butadiene) is mainly obtained by extracting from a C5 fraction obtained by cracking of naphtha. However in recent years, with the use of light feed crackers, an amount of produced isoprene tends to decrease and its supply is concerned. Also in recent years, since variation of oil price influences greatly, it is requested to establish a system in which isoprene derived from non-oil sources is produced inexpensively in order to ensure the stable supply of isoprene.

Concerning such a demand, a method in which isoprene is produced by a fermentation method using an inducer and a microorganism to which an ability to produce isoprene was given has been known (see WO2009/076676, which is incorporated herein by reference in its entirety).

A method utilizing an environmental factor such as light or temperature in place of the inducer has been also known. For example, a method of producing isoprene utilizing light (see Pia Lindberg, Sungsoon Park, Anastasios Melis, Metabolic Engineering 12 (2010): 70-79, which is incorporated herein by reference in its entirety) and a method of producing an objective protein (e.g., interferon-γ, insulin) utilizing temperature (see Norma A Valdez-Cruz, Luis Caspeta, Néstor O Pérez, Octavio T Ramirez, Mauricio A Trujillo-Roldán, Microbial Cell Factories 2010, 9: 18, which is incorporated herein by reference in its entirety) have been known.

As described in WO2009/076676; Pia Lindberg, Sungsoon Park, Anastasios Melis, Metabolic Engineering 12 (2010): 70-79; Norma A Valdez-Cruz, Luis Caspeta, Néstor O Pérez, Octavio T Ramirez, Mauricio A Trujillo-Roldán, Microbial Cell Factories 2010, 9: 18; TL Sivy, R Fall, T N Rosenstiel, Biosci. Biotechnol. Biochem., January 2011; 75(12): 2376-83; and Martin V, Pitera D, Withers S, Newman J, Keasling J, Nat. Biotechnol., 21, 796-802, 2003, all of which are incorporated herein by reference in their entireties, when isoprenoid compounds including isoprene are produced in a microorganism, a mevalonate (MVA) pathway or a methylerythritol (MEP) pathway is utilized. If a metabolic flux in the MVA pathway is increased in order to enhance a quantity of production of isoprenoid compounds, growth of a microorganism may be inhibited (see TL Sivy, R Fall, T N Rosenstiel, Biosci. Biotechnol. Biochem., January 2011; 75(12): 2376-83 and Martin V, Pitera D, Withers S, Newman J, Keasling J, Nat. Biotechnol., 21, 796-802, 2003). A method of separating a growth phase of the microorganism from a formation phase of a substance can be effective to avoid growth of the microorganism. A concept that a period for the growth of a microorganism is separated from a period for producing a substance has been known in the production of the substance by a fermentation method (see Fermentation Handbook edited by Working Group for Fermentation and Metabolism, Japan Bioindustry Association, 2001, which is incorporated herein by reference in its entirety). To accomplish this concept, a way to transfer from the growth phase of the microorganism to the formation phase of the substance and a way to continue to produce the substance in the formation phase of the substance are required. An example in which a microorganism that produces isoprene by the addition of an inducer is grown under a condition with no addition of the inducer and a sufficient amount of microbial cells are acquired followed by adding the inducer to form isoprene has been known in isoprene fermentation (see WO2009/076676). IPTG (Isopropyl β-D-1-thiogalactopyranoside), tetracycline and the like can be utilized as the inducer. When IPTG or tetracycline is present in a culture tank, a microorganism continues to have an activity to produce isoprene, but before long, IPTG or tetracycline is decomposed and consequently the microorganism becomes less able to keep the activity to produce isoprene. Thus, IPTG or tetracycline must be added continuously. However, these inducers are expensive and increase production cost of isoprene. Therefore, it is desirable to construct a production process not using the inducer.

A technique for inducing the production of isoprene by using a promoter, a transcription activity of which is increased under strong light and increasing light intensity has been reported in blue-green algae and the like (see Pia Lindberg, Sungsoon Park, Anastasios Melis, Metabolic Engineering 12 (2010): 70-79) as a method of transferring to the formation phase of isoprene without using the inducer. Microorganisms in which this method is available are limited to microorganisms having a metabolic switch function of a light response type and microorganisms that can perform photosynthesis.

As an example of producing a substance without using the inducer, which is the case different from isoprene, it has been reported that when a protein such as interferon-γ or insulin is produced in Escherichia coli by the fermentation method, elevation of culture temperature (from 30° C. to 42° C.) enables the transfer from the growth phase of the microorganism to the formation phase of the protein (see Norma A Valdez-Cruz, Luis Caspeta, Néstor O Pérez, Octavio T Ramirez, Mauricio A Trujillo-Roldán, Microbial Cell Factories 2010, 9: 18). While this method is effective for the production of the protein, the culture temperature at 42° C. differs from the temperature at which an optimal metabolic rate of E. coli is obtained thus reducing a consumption rate of a substrate such as glucose. If this technique is applied to the isoprene fermentation, a fermentation process uses two culture temperature conditions. It is required therefore to culture under a condition that differs from microbial cell growth and the optimal metabolic rate of a host at which isoprene fermentation is utilized. As a result, throughout the fermentation production processes, it is concerned that the isoprene productivity under the culture condition at high temperature is reduced compared with a fermentation production process in which the microorganism is cultured at temperature at which the optimal metabolic rate is obtained.

Thus, as described above, there remains a need for a method of transferring the microorganism to the formation phase of isoprene without using an inducer and a method of keeping the activity to produce the isoprene in the formation phase thereof.

### SUMMARY OF THE INVENTION

Accordingly, it is one object of the present invention to provide novel methods for producing an isoprenoid compound such as isoprene.

It is another object of the present invention to provide novel methods for producing an isoprenoid compound such as isoprene by fermentation of a microorganism, in which the microorganism is transferred to the formation phase of isoprene without using an inducer.

It is another object of the present invention to provide novel methods for producing an isoprenoid compound such as isoprene by fermentation of a microorganism, in which the activity of producing the isoprenoid compound is maintained in the formation phase.

These and other objects, which will become apparent during the following detailed description, have been achieved by the inventors' discovery that an isoprenoid compound such as an isoprene can be efficiently formed by first growing an isoprenoid compound-forming microorganism under the sufficient concentration of a growth promoting agent and then reducing the concentration of the growth promoting agent. The present inventors have also found that the formation of the isoprenoid compound can be attained under a culture control condition where an oxygen consumption rate of a fermenting microorganism is nearly equal to an oxygen supply rate to a fermentation jar, and when phosphate is present at a certain concentration or below in a culture medium.

Thus, the present invention provides the following:

(1) A method of producing an isoprenoid compound, comprising:

1) culturing an isoprenoid compound-forming microorganism in the presence of a growth promoting agent at a sufficient concentration to grow the isoprenoid compound-forming microorganism;

2) decreasing the sufficient concentration of the growth promoting agent to induce formation of the isoprenoid compound by the isoprenoid compound-forming microorganism; and

3) culturing the isoprenoid compound-forming microorganism to form the isoprenoid compound.

(2) The method described above, wherein the isoprenoid compound-forming microorganism has an ability to form the isoprenoid compound depending on a promoter which is inversely dependent on the growth promoting agent.

(3) The method described above, wherein the growth promoting agent is oxygen.

(4) The method described above, wherein the isoprenoid compound-forming microorganism is an aerobic microorganism.

(5) The method described above, wherein the promoter is a microaerobically inducible promoter.

(6) The method described above, wherein the microaerobically inducible promoter is a promoter of the gene encoding a lactate dehydrogenase, or a promoter of the gene encoding an α-acetolactate decarboxylase.

(7) The method described above, wherein the isoprenoid compound is a monoterpene, and the isoprenoid compound-forming microorganism is a monoterpene-forming microorganism.

(8) The method described above, wherein the isoprenoid compound is a limonene, and the isoprenoid compound-forming microorganism is a limonene-forming microorganism.

(9) The method described above, wherein the isoprenoid compound is a linanol, and the isoprenoid compound-forming microorganism is a linanol-forming microorganism.

(10) The method described above, wherein the isoprenoid compound is an isoprene monomer, and the isoprenoid compound-forming microorganism is an isoprene-forming microorganism.

(11) A method of producing an isoprenoid compound, comprising:

1′) supplying oxygen into a liquid phase containing the isoprenoid compound-forming microorganism in a system comprising a gas phase and the liquid phase to grow the isoprenoid compound-forming microorganism in the presence of dissolved oxygen at a sufficient concentration;

2′) decreasing the dissolved oxygen concentration in the liquid phase to induce formation of the isoprenoid compound by the isoprenoid compound-forming microorganism in the liquid phase;

3′) culturing the isoprenoid compound-forming microorganism in the liquid phase to form the isoprenoid compound; and

4′) collecting the isoprenoid compound.

(12) The method described above, wherein the isoprenoid compound is an isoprene.

(13) The method described above, wherein an oxygen concentration in the gas phase is controlled depending on the dissolved oxygen concentration in the liquid phase, and wherein the dissolved oxygen concentration in the liquid phase is decreased to avoid isoprene burst in the gas phase.

(14) The method described above, wherein the dissolved oxygen concentration in the liquid phase is 0.34 ppm or less in 2′) and 3′).

(15) The method described above, wherein an isoprene-forming rate in an induction phase is 80 ppm/vvm/h or less.

(16) The method described above, wherein the growth promoting agent is a phosphorus compound.

(17) The method described above, wherein the promoter is a phosphorus deficiency-inducible promoter.

(18) The method described above, wherein the phosphorus deficiency-inducible promoter is a promoter of the gene encoding an acid phosphatase, or a promoter of the gene encoding a phosphorus uptake carrier.

(19) The method described above, wherein formation of the isoprenoid by the isoprenoid-forming microorganism is induced in the presence of a total phosphorus at concentration of 50 mg/L or less.

(20) The method described above, wherein the isoprenoid-forming rate in the induction phase is 600 ppm/vvm/h or less.

(21) The method described above, wherein the isoprenoid compound is an isoprene.

(22) The method described above, wherein the isoprenoid compound-forming microorganism has an ability to synthesize dimethylallyl diphosphate via a methylerythritol phosphate pathway.

(23) The method described above, wherein the isoprenoid compound-forming microorganism has the ability to synthesize dimethylallyl diphosphate via a mevalonate pathway.

(24) The method described above, wherein the isoprenoid compound-forming microorganism is a microorganism belonging to the family Enterobacteriaceae.

(25) The method described above, wherein the isoprenoid compound-forming microorganism is a microorganism belonging to the genus Pantoea, Enterobacter, or Escherichia.

(26) The method described above, wherein the isoprenoid compound-forming microorganism is Pantoea ananatis, Enterobacter aerogenes or Escherichia coli.

(27) A method of producing an isoprene polymer comprising:

(I) forming the isoprene monomer by the method according to any one of claims 10 to 26; and

(II) polymerizing the isoprene monomer to form the isoprene polymer.

(28) A polymer derived from isoprene produced by the method described above.

(29) A rubber composition comprising the polymer described above.

(30) A tire manufactured by using the rubber composition described above.

### Effect of the Invention

According to the present invention, it is possible to produce an isoprenoid compound inexpensively by separating the growth phase of the microorganism and the formation phase of the isoprenoid compound without using an expensive inducer.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 indicates measurement with time of dissolved oxygen (DO) concentrations in cultivations of an arabinose-inducible isoprenoid compound-forming microorganism (GI08/Para) and microaerobically inducible isoprenoid compound-forming microorganism (GI08/Para);

FIG. 2A and FIG. 2B indicate growth (FIG. 2A) and amount of formed isoprene (FIG. 2B) (mg/batch) in cultivations of an arabinose-inducible isoprenoid compound-forming microorganism (GI08/Para) and a microaerobically inducible isoprenoid compound-forming microorganism (GI08/Para);

FIG. 3 indicates measurement with time of the dissolved oxygen (DO) concentrations in cultivation of microaerobically inducible isoprenoid compound-forming microorganisms (GI08/Para);

FIG. 4A and FIG. 4B indicate the growth (FIG. 4A) and amount of formed isoprene (FIG. 4B) (mg/batch) in cultivation of microaerobically inducible isoprenoid compound-forming microorganisms (GI08/Para) under various concentration conditions of dissolved oxygen;

FIG. 5 indicates a pAH162-Para-mvaES plasmid possessing an mvaES operon derived from E. faecalis under control of E. coli Para promoter and a repressor gene araC;

FIG. 6 indicates a map of pAH162-mvaES;

FIG. 7 indicates a plasmid for chromosome fixation of pAH162-MCS-mvaES.

FIG. 8A, FIG. 8B, and FIG. 8C indicate a set of plasmids for chromosome fixation which possess an mvaES gene under transcription control of (FIG. 8A) P_{lldD}, (FIG. 8B) P_{phoC}, or (FIG. 8C) P_{pstS};

FIG. 9 indicates an outline for construction of a pAH162-λattL-KmR-λattR vector;

FIG. 10 indicates a pAH162-Ptac expression vector for chromosome fixation;

FIG. 11 indicates codon optimization in a KDyI operon obtained by chemical synthesis;

FIG. 12A and FIG. 12B indicate plasmids pAH162-Tc-Ptac-KDyI (FIG. 12A) and pAH162-Km-Ptac-KDyI (FIG. 12B) for chromosome fixation, which retain the KDyI operon with codon optimization;

FIG. 13 indicates a plasmid for chromosome fixation, which retains a mevalonate kinase gene derived from M. paludicola;

FIG. 14A, FIG. 14B, and FIG. 14C indicate maps of genome modifications of (FIG. 14A) ΔampC::attBₚₕᵢ₈₀, (FIG. 14B) ΔampH::attBₚₕᵢ₈₀, and (FIG. 14C) Δcrt::attBₚₕᵢ₈₀;

FIG. 15A and FIG. 15B indicate maps of genome modifications of (FIG. 15A) Δcrt::pAH162-P_{tac}-mvk(X) and (FIG. 15B) Δcrt::P_{tac}-mvk(X);

FIG. 16A, FIG. 16B, and FIG. 16C indicate maps of chromosome modifications of (FIG. 16A) ΔampH::pAH162-Km-P_{tac}-KDyI, (FIG. 16B) ΔampC::pAH162-Km-P_{tac}-KDyI and (FIG. 16C) ΔampC::P_{tac}-KDyI;

FIG. 17A and FIG. 17B indicate maps of chromosome modifications of (FIG. 17A) ΔampH::pAH162-Px-mvaES and (FIG. 17B) ΔampC::pAH162-Px-mvaES;

FIG. 18 indicates measurement with time of phosphorus concentrations in cultures of an arabinose inducible isoprenoid compound-forming microorganism (SWITCH-Para/ispSM) and a phosphorus deficient isoprenoid compound-forming microorganism (SWITCH-PphoC/ispSM, SWITCH-PpstS/ispSM);

FIG. 19A and FIG. 19B indicate growth (FIG. 19A) and amounts of produced isoprene (FIG. 19B) (mg/batch) in cultures of an arabinose inducible isoprenoid compound-forming microorganism (SWITCH-Para/ispSM) and a phosphorus deficient isoprenoid compound-forming microorganism (SWITCH-PphoC/ispSM, SWITCH-PpstS/ispSM);

FIG. 20 indicates isoprene concentrations (ppm) of fermentation gas in cultures of an arabinose inducible isoprenoid compound-forming microorganism (SWITCH-Para/ispSM) and a phosphorus deficient type isoprenoid compound-forming microorganism (SWITCH-PphoC/ispSM, SWITCH-PpstS/ispSM);

FIG. 21 indicates measurement with time of dissolved oxygen (DO) concentrations in cultures of the arabinose inducible isoprenoid compound-forming microorganism (SWITCH-Para/ispSM) and a microaerobically inducible isoprenoid compound-forming microorganism (SWITCH-lld/ispSM);

FIG. 22A and FIG. 22B indicate growth (FIG. 22A) amounts of formed isoprene (FIG. 22B) (mg/batch) in cultures of an arabinose inducible isoprenoid compound-forming microorganism (SWITCH-Para/ispSM) and a microaerobically inducible isoprenoid compound-forming microorganism (SWITCH-lld/ispSM);

FIG. 23 indicates isoprene concentrations (ppm) of fermentation gas in cultures of an arabinose inducible isoprenoid compound-forming microorganism (SWITCH-Para/ispSM) and a microaerobically inducible isoprenoid compound-forming microorganism (SWITCH-lld/ispSM);

FIG. 24 indicates a burst limit of isoprene and a critical oxygen concentration in a gas phase (headspace) (extract from U.S. Pat. No. 8,420,360B2, which is incorporated herein by reference in its entirety);

FIG. 25A and FIG. 25B indicate growth (FIG. 25A) and amounts of formed isoprene (FIG. 25B) (mg/batch) in cultures of a phosphorus deficient type isoprenoid compound-forming microorganism in which glucose dehydrogenase (gcd) gene is disrupted (SWITCH-PphoC Δgcd/ispSM); and

FIG. 26 indicates changes in accumulated gluconate in culture broth over time in a phosphorus deficient type isoprenoid compound-forming microorganism in which gcd gene is disrupted (SWITCH-PphoC Δgcd/ispSM).

### DETAILED DESCRIPTION OF THE PREFERRED EMBODIMENTS

The present invention provides a method of producing an isoprenoid compound.

The isoprenoid compound includes one or more isoprene units which have the molecular formula (C₅H₈)ₙ. The precursor of the isoprene unit may be isopentenyl pyrophosphate or dimethylallyl pyrophosphate. More than 30,000 kinds of isoprenoid compounds have been identified and new compounds have been identified. Isoprenoids are also known as terpenoids. The difference between terpenes and terpenoids is that terpenes are hydrocarbons, whereas terpenoids may contain additional functional groups. Terpenes are classified by the number of isoprene units in the molecule: hemiterpenes (C5), monoterpenes (C10), sesquiterpenes (C15), diterpenes (C20), sesterterpenes (C25), triterpenes (C30), sesquarterpenes (C35), tetraterpenes (C40), polyterpenes, norisoprenoids, for example. Examples of monoterpenes include pinene, nerol, citral, camphor, menthol, limonene, and linalool. Examples of sesquiterpenes include nerolidol and farnesol. Examples of diterpenes include phytol and vitamin A1. Squalene is an example of a triterpene, and carotene (provitamin A1) is a tetraterpene (see Nature Chemical Biology 2, 674-681 (2006), Nature Chemical Biology 5, 283-291 (2009) Nature Reviews Microbiology 3, 937-947 (2005), Adv Biochem Eng Biotechmol (DOI: 10.1007/10_2014_288, all of which are incorporated herein by reference in their entireties). Preferably, the isoprenoid compound is an isoprene (monomer).

The method of the present invention comprises the following 1) to 3):

1) culturing an isoprenoid compound-forming microorganism in the presence of a growth promoting agent at a sufficient concentration to grow the isoprenoid compound-forming microorganism;

2) decreasing the concentration of the growth promoting agent to induce formation of the isoprenoid compound by the isoprene-forming microorganism; and

3) culturing the isoprenoid compound-forming microorganism to form the isoprenoid compound.

IPP (isopentenyl diphosphate) or DMAPP (dimethylallyl diphosphate) that is a substrate of isoprene synthesis has been known to be a precursor of peptide glycan and an electron acceptor, such as menaquinone and the like, and to be essential for growth of microorganisms (see Fujisaki et al., J. Biochem., 1986; 99: 1137-1146, which is incorporated herein by reference in its entirety.). In the method of the present invention, in the light of efficient production of an isoprenoid compound, step 1) corresponding to a growth phase of a microorganism and step 3) corresponding to a formation phase of the isoprenoid compound are separated. The method also comprises step 2) corresponding to an induction phase of isoprenoid compound formation for transferring the growth phase of the microorganism to the formation phase of the isoprenoid compound.

In the present invention, the growth promoting agent refer to a factor essential for the growth of a microorganism or a factor having an activity of promoting the growth of the microorganism, which can be consumed by the microorganism, the consumption of which causes reduction of its amount in a culture medium, consequently lost or reduction of the growth of the microorganism. For example, when the growth promoting agent in a certain amount is used, a microorganism continues to grow until the growth promoting agent in that amount is consumed, but once the growth promoting agent is entirely consumed, the microorganism cannot grow or the growth rate can decrease. Therefore, the degree of the growth of the microorganism can be regulated by the growth promoting agent. Examples of such a growth promoting agent may include substances such as oxygen (gas); minerals such as ions of iron, magnesium, potassium and calcium; phosphorus compounds such as monophosphoric acid, diphosphoric acid and polyphosphoric acid, or salt thereof; nitrogen compounds such as ammonia, nitrate, nitrite, nitrogen (gas), and urea; sulfur compounds such as ammonium sulfate and thiosulfuric acid; and nutrients such as vitamins (e.g., vitamin A, vitamin D, vitamin E, vitamin K, vitamin B1, vitamin B2, vitamin B6, vitamin B12, niacin, pantothenic acid, biotin, ascorbic acid), and amino acids (e.g., alanine, arginine, asparagine, aspartic acid, cysteine, glutamine, glutamic acid, glycine, histidine, leucine, isoleucine, lysine, methionine, phenylalanine, proline, serine, threonine, tryptophan, tyrosine, valine, selenocysteine). One growth promoting agent may be used or two or more growth promoting agents may be used in combination in the method of the present invention.

In the present invention, the isoprenoid compound-forming microorganism refers to a microorganism having an ability to grow depending on the growth promoting agent and an ability to form an isoprenoid compound depending on a promoter which is inversely dependent on the growth promoting agent, to which an ability to synthesize the isoprenoid compound by an enzymatic reaction has been given. The isoprenoid compound-forming microorganism can grow in the presence of the growth promoting agent at concentration sufficient for the growth of the isoprenoid compound-forming microorganism. Here, the “sufficient concentration” can refer to that the growth promoting agent is used at concentration which is effective for the growth of the isoprenoid compound-forming microorganism. The expression “ability to form an(the) isoprenoid compound depending on a promoter which is inversely depending on the growth promoting agent” can mean that the isoprenoid compound cannot be formed or a forming efficiency of the isoprenoid compound is low in the presence of the growth promoting agent at relatively high concentration whereas the isoprenoid compound can be formed or the forming efficiency of the isoprenoid compound is high in the presence of the growth promoting agent at relatively low concentration or in the absence of the growth promoting agent. Therefore, the isoprenoid compound-forming microorganism used in the present invention can grow well but cannot form the isoprenoid compound or exhibits low forming efficiency of the isoprenoid compound in the presence of the growth promoting agent at sufficient concentration. The isoprenoid compound-forming microorganism cannot grow well but can form the isoprenoid compound and exhibits high forming efficiency of the isoprenoid compound in the presence of the growth promoting agent at insufficient concentration or in the absence of the growth promoting agent. Preferably, the isoprenoid compound-forming microorganism is an isoprene-forming microorganism.

In such an isoprenoid compound-forming microorganism, a gene encoding an isoprenoid compound-synthetic enzyme can be present under the control of a promoter which is inversely dependent on the growth promoting agent. The expression “promoter which is inversely dependent on the growth promoting agent” can mean a promoter not having at all or having low transcription activity in the presence of the growth promoting agent at relatively high concentration but having some or high transcription activity in the presence of the growth promoting agent at relatively low concentration or in the absence of the growth promoting agent. Therefore, the promoter which is inversely dependent on the growth promoting agent can suppress the expression of the gene encoding an isoprenoid compound-synthetic enzyme in the presence of the growth promoting agent at a concentration sufficient for the growth of the isoprenoid compound-forming microorganism whereas it can promote the expression of the gene encoding an isoprenoid compound-synthetic enzyme in the presence of the growth promoting agent at the concentration insufficient for the growth of the isoprenoid compound-forming microorganism or in the absence of the growth promoting agent. Specifically, the isoprenoid compound-forming microorganism is a microorganism transformed with an expression vector comprising the gene encoding an isoprenoid compound-synthetic enzyme present under the control of the promoter which is inversely dependent on the growth promoting agent. The gene encoding an isoprenoid compound-synthetic enzyme refers to one or more genes encoding one or more enzymes involved in a synthesis of an isoprenoid compound. Examples of the gene encoding an isoprenoid compound-synthetic enzyme include an isoprene synthase gene, geranyl diphosphate synthase gene, a farnesyl diphosphate synthase gene, a linalool synthase gene, an amorpha-4,11-diene synthase gene, a beta-caryophyllene synthase gene, a germacrene A synthase gene, a 8-epicedrol synthase gene, a valencene synthase gene, a (+)-delta-cadinene synthase gene, a germacrene C synthase gene, a (E)-beta-farnesene synthase gene, a casbene synthase gene, a vetispiradiene synthase gene, a 5-epi-aristolochene synthase gene, an aristolochene synthase gene, an alpha-humulene synthase gene, an (E,E)-alpha-farnesene synthase gene, a (−)-beta-pinene synthase gene, a gamma-terpinene synthase gene, a limonene cyclase gene, a linalool synthase gene, a 1,8-cineole synthase gene, a (+)-sabinene synthase gene, an E-alpha-bisabolene synthase gene, a (+)-bornyl diphosphate synthase gene, a levopimaradiene synthase gene, an abietadiene synthase gene, an isopimaradiene synthase gene, a (E)-gamma-bisabolene synthase gene, a taxadiene synthase gene, a copalyl pyrophosphate synthase gene, a kaurene synthase gene, a longifolene synthase gene, a gamma-humulene synthase gene, a Delta-selinene synthase gene, a beta-phellandrene synthase gene, a limonene synthase gene, a myrcene synthase gene, a terpinolene synthase gene, a (−)-camphene synthase gene, a (+)-3-carene synthase gene, a syn-copalyl diphosphate synthase gene, an alpha-terpineol synthase gene, a syn-pimara-7,15-diene synthase gene, an ent-sandaracopimaradiene synthase gene, a sterner-13-ene synthase gene, a E-beta-ocimene gene, a S-linalool synthase gene, a geraniol synthase gene, an epi-cedrol synthase gene, an alpha-zingiberene synthase gene, a guaiadiene synthase gene, a cascarilladiene synthase gene, a cis-muuroladiene synthase gene, an aphidicolan-16b-ol synthase gene, an elizabethatriene synthase gene, a santalol synthase gene, a patchoulol synthase gene, a zinzanol synthase gene, a cedrol synthase gene, a sclareol synthase gene, a copalol synthase gene, a manool synthase gene, a limonene monooxygenase gene, a carveol dehydrogenase gene, and the isoprene synthase gene, geranyl diphosphate synthase gene, farnesyl diphosphate synthase gene, linalool synthase gene and limonene synthase gene are preferred.

For example, when the growth promoting agent is oxygen, a microaerobically inducible promoter can be utilized. The microaerobically inducible promoter can refer to a promoter that can promote the expression of a downstream gene under a microaerophilic condition. In general, the saturated concentration of dissolved oxygen is 7.22 ppm (under the air condition: 760 mmHg, 33° C., 20.9% oxygen and saturated water vapor). The microaerophilic condition can refer to a condition where a (dissolved) oxygen concentration is 0.35 ppm or less. The (dissolved) oxygen concentration under the microaerophilic condition may be 0.30 ppm or less, 0.25 ppm or less, 0.20 ppm or less, 0.15 ppm or less, 0.10 ppm or less, or 0.05 ppm or less. Examples of the microaerobically inducible promoter may include a promoter of the gene encoding a D- or L-lactate dehydrogenase (e.g., lld, ldhA), a promoter of the gene encoding an alcohol dehydrogenase (e.g., adhE), a promoter of the gene encoding a pyruvate formate lyase (e.g., pflB), and a promoter of the gene encoding an α-acetolactate decarboxylase (e.g., budA).

When the growth promoting agent is a phosphorus compound, a phosphorus deficiency-inducible promoter can be utilized. The expression “phosphorus deficiency-inducible promoter” can refer to a promoter that can promote the expression of a downstream gene at low concentration of phosphorus compound. The low concentration of phosphorus compound can refer to a condition where a (free) phosphorus concentration is 100 mg/L or less. The expression “phosphorus” is synonymous to the expression “phosphorus compound”, and they can be used in exchangeable manner. The concentration of total phosphorus is able to quantify by decomposing total kinds of phosphorus compounds in liquid to phosphorus in the form of orthophosphoric acid by strong acid or oxidizing agent. The total phosphorus concentration under a phosphorus deficient condition may be 50 mg/L or less, 10 mg/L or less, 5 mg/L or less, 1 mg/L or less, 0.1 mg/L or less, or 0.01 mg/L or less. Examples of the phosphorus deficiency-inducible promoter may include a promoter of the gene encoding alkali phosphatase (e.g., phoA), a promoter of the gene encoding an acid phosphatase (e.g., phoC), a promoter of the gene encoding a sensor histidine kinase (phoR), a promoter of the gene encoding a response regulator (e.g., phoB), and a promoter of the gene encoding a phosphorus uptake carrier (e.g., pstS).

When the growth promoting agent is an amino acid, an amino acid deficiency-inducible promoter can be utilized. The amino acid deficiency-inducible promoter can refer to a promoter that can promote the expression of a downstream gene at low concentration of an amino acid. The low concentration of the amino acid can refer to a condition where a concentration of a (free) amino acid or a salt thereof is 100 mg/L or less. The concentration of the (free) amino acid or a salt thereof under the amino acid deficient condition may be 50 mg/L or less, 10 mg/L or less, 5 mg/L or less, 1 mg/L or less, 0.1 mg or less or 0.01 mg/L or less. Examples of the amino acid deficiency-inducible promoter may include a promoter of the gene encoding a tryptophan leader peptide (e.g., trpL) and a promoter of the gene encoding an N-acetylglutamate synthase (e.g., ArgA).

The isoprenoid compound-forming microorganism can be obtained by transforming a host microorganism with a vector for expressing the isoprenoid compound-synthetic enzyme such as the isoprene synthase. Examples of the isoprene synthase may include the isoprene synthase derived from kudzu (Pueraria montana var. lobata), poplar (Populus alba×Populus tremula), mucuna (Mucuna bracteata), willow (Salix), false acacia (Robinia pseudoacacia), Japanese wisteria (Wisterria), eucalyptus (Eucalyptus globulus), and tea plant (Melaleuca alterniflora) (see, e.g., Evolution 67 (4), 1026-1040 (2013), which is incorporated herein by reference in its entirety). The vector for expressing the isoprenoid compound-synthetic enzyme may be an integrative vector or a non-integrative vector. In the expression vector, the gene encoding the isoprenoid compound-synthetic enzyme may be placed under the control of the promoter which is inversely dependent on the growth promoting agent.

The phrase “derived from” as used herein for a nucleic acid sequence such as a gene, a promoter, and the like, or an amino acid sequence such as a protein, can mean a nucleic acid sequence or an amino acid sequence that are naturally or natively synthesized by a microorganism or can be isolated from the natural or wild-type microorganism.

The isoprenoid compound-forming microorganism may further express a mevalonate kinase in addition to the isoprenoid compound-synthetic enzyme. Therefore, the isoprenoid compound-forming microorganism may be transformed with a vector for expressing the mevalonate kinase. Examples of the mevalonate kinase gene may include genes from microorganisms belonging to the genus Methanosarcina such as Methanosarcina mazei, the genus Methanocella such as Methanocella paludicola, the genus Corynebacterium such as Corynebacterium variabile, the genus Methanosaeta such as Methanosaeta concilii, and the genus Nitrosopumilus such as Nitrosopumilus maritimus. The vector for expressing the mevalonate kinase may be an integrative vector or a non-integrative vector. In the expression vector, the gene encoding the mevalonate kinase may be placed under the control of a constitutive promoter or inducible promoter (e.g., the promoter which is inversely dependent on the growth promoting agent). Specifically, the gene encoding the mevalonate kinase may be placed under the control of the constitutive promoter. Examples of the constitutive promoter include the tac promoter, the lac promoter, the trp promoter, the trc promoter, the T7 promoter, the T5 promoter, the T3 promoter, and the SP6 promoter.

DMAPP, that is a precursor of the isoprenoid compound (e.g., a substrate of isoprene synthesis), is typically biosynthesized via either a methylerythritol phosphate pathway or a mevalonate pathway inherently or natively possessed by a microorganism. Therefore, in the light of DMAPP supply for efficiently producing the isoprenoid compound, the methylerythritol phosphate pathway and/or the mevalonate pathway may be enhanced in the isoprenoid compound-forming microorganism used in the present invention, as described later.

The isoprenoid compound-forming microorganism used in the present invention as a host can be a bacterium or a fungus. The bacterium may be a gram-positive bacterium or a gram-negative bacterium. The isoprenoid compound-forming microorganism can be a microorganism belonging to the family Enterobacteriaceae, and particularly preferably a microorganism belonging to the family Enterobacteriaceae among microorganisms described later.

Examples of the gram-positive bacterium may include bacteria belonging to the genera Bacillus, Listeria, Staphylococcus, Streptococcus, Enterococcus, Clostridium, Corynebacterium, and Streptomyces. Bacteria belonging to the genera Bacillus and Corynebacterium are preferable. Examples of the bacteria belonging to the genus Bacillus may include Bacillus subtilis, Bacillus anthracis, and Bacillus cereus. Bacillus subtilis is more preferable. Examples of the bacteria belonging to the genus Corynebacterium may include Corynebacterium glutamicum, Corynebacterium efficiens, and Corynebacterium callunae. Corynebacterium glutamicum is more preferable.

Examples of the gram-negative bacterium may include bacteria belonging to the genera Escherichia, Pantoea, Salmonella, Vibrio, Serratia, and Enterobacter. The bacteria belonging to the genera Escherichia, Pantoea and Enterobacter are preferable.

Escherichia coli is preferable as the bacterium belonging to the genus Escherichia.

Examples of the bacteria belonging to the genus Pantoea may include Pantoea ananatis, Pantoea stewartii, Pantoea agglomerans, and Pantoea citrea. Pantoea ananatis and Pantoea citrea are preferable. Strains exemplified in the European Patent Application Publication EP 0 952 221, which is incorporated herein by reference in its entirety, may be used as the bacteria belonging to the genus Pantoea. Examples of representative strains of the bacteria belonging to the genus Pantoea may include Pantoea ananatis AJ13355 strain (FERM BP-6614), Pantoea ananatis AJ13356 strain (FERM BP-6615) disclosed in the European Patent Application Publication EP 0952 221, Pantoea ananatis SC17 strain (FERMBP-11902), and Pantoea ananatis SC17(0) strain (VKPM B-9246; Katashikina J I et al., BMC Mol Biol 2009; 10:34, which is incorporated herein by reference in its entirety).

Examples of the bacteria belonging to the genus Enterobacter may include Enterobacter agglomerans and Enterobacter aerogenes. Enterobacter aerogenes is preferable as the bacterium belonging to the genus Enterobacter. The bacterial strains exemplified in the European Patent Application Publication EP 0 952 221 may be used as the bacteria belonging to the genus Enterobacter. Examples of representative strains of the bacteria belonging to the genus Enterobacter may include Enterobacter agglomerans ATCC12287 strain, Enterobacter aerogenes ATCC13048 strain, Enterobacter aerogenes NBRC12010 strain (Biotechnol. Bioeng., 2007 Mar. 27; 98(2) 340-348, which is incorporated herein by reference in its entirety), Enterobacter aerogenes AJ110637 (FERM BP-10955), and the like. The Enterobacter aerogenes AJ110637 strain was deposited to International Patent Organism Depositary (IPOD), National Institute of Advanced Industrial Science and Technology (AIST) (Chuo No. 6, Higashi 1-1-1, Tsukuba City, Ibaraki Pref., JP, Postal code 305-8566; currently, International Patent Organism Depositary, National Institute of Technology and Evaluation (IPOD NITE), #120, 2-5-8 Kazusakamatari, Kisarazu-shi, Chiba, 292-0818, Japan) as of Aug. 22, 2007, and was transferred to the international deposition based on the Budapest Treaty on Mar. 13, 2008, and the deposit number FERM BP-10955 was given thereto.

Examples of the fungus may include microorganisms belonging to the genera Saccharomyces, Schizosaccharomyces, Yarrowia, Trichoderma, Aspergillus, Fusarium, and Mucor. The microorganisms belonging to the genera Saccharomyces, Schizosaccharomyces, Yarrowia, or Trichoderma are preferable.

Examples of the microorganisms belonging to the genus Saccharomyces may include Saccharomyces carlsbergensis, Saccharomyces cerevisiae, Saccharomyces diastaticus, Saccharomyces douglasii, Saccharomyces kluyveri, Saccharomyces norbensis, and Saccharomyces oviformis. Saccharomyces cerevisiae is preferable as the fungus belonging to the genus Saccharomyces.

Schizosaccharomyces pombe is preferable as the microorganisms belonging to the genus Schizosaccharomyces.

Yarrowia lypolytica is preferable as the microorganisms belonging to the genus Yarrowia.

Examples of the microorganisms belonging to the genus Trichoderma may include Trichoderma harzianum, Trichoderma koningii, Trichoderma longibrachiatum, Trichoderma reesei, and Trichoderma viride. Trichoderma reesei is preferable.

The pathway to synthesize dimethylallyl diphosphate (DMAPP) that is a precursor of the isoprenoid compound (e.g., the substrate of the isoprene synthase) may further be enhanced in the isoprene-forming microorganism. For such an enhancement, an expression vector that expresses an isopentenyl-diphosphate delta isomerase having an ability to convert isopentenyl diphosphate (IPP) into dimethylallyl diphosphate (DMAPP) may be introduced into the isoprenoid compound-forming microorganism. An expression vector that expresses one or more enzymes involved in the mevalonate pathway and/or methylerythritol phosphate pathway associated with formation of IPP and/or DMAPP may also be introduced into the isoprenoid compound-forming microorganism. The expression vector for such an enzyme may be an integrative vector or a non-integrative vector. The expression vector for such an enzyme may express further a plurality of enzymes (e.g., one or more, two or more, three or more or four or more) involved in the mevalonate pathway and/or the methylerythritol phosphate pathway, and may be, for example, an expression vector for polycistronic mRNA. Origin of one or more enzymes involved in the mevalonate pathway and/or the methylerythritol phosphate pathway may be homologous or heterologous to the host. When the origin of the enzyme involved in the mevalonate pathway and/or the methylerythritol phosphate pathway is heterologous to the host, for example, the host may be a bacterium as described above (e.g., Escherichia coli) and the enzyme involved in the mevalonate pathway may be derived from a fungus (e.g., Saccharomyces cerevisiae). In addition, when the host inherently produces the enzyme involved in the methylerythritol phosphate pathway, an expression vector to be introduced into the host may express an enzyme involved in the mevalonate pathway.

Examples of the isopentenyl-diphosphate delta isomerase (EC: 5.3.3.2) may include Idi1p (ACCESSION ID NP_015208), AT3G02780 (ACCESSION ID NP_186927), AT5G16440 (ACCESSION ID NP_197148) and Idi (ACCESSION ID NP_417365). In the expression vector, the gene encoding the isopentenyl-diphosphate delta isomerase may be placed under the control of the promoter which is inversely dependent on the growth promoting agent.

Examples of the enzymes involved in the mevalonate (MVA) pathway may include mevalonate kinase (EC: 2.7.1.36; example 1, Erg12p, ACCESSION ID NP_013935; example 2, AT5G27450, ACCESSION ID NP_001190411), phosphomevalonate kinase (EC: 2.7.4.2; example 1, Erg8p, ACCESSION ID NP_013947; example 2, AT1G31910, ACCESSION ID NP_001185124), diphosphomevalonate decarboxylase (EC: 4.1.1.33; example 1, Mvd1p, ACCESSION ID NP_014441; example 2, AT2G38700, ACCESSION ID NP_181404; example 3, AT3G54250, ACCESSION ID NP_566995), acetyl-CoA-C-acetyltransferase (EC: 2.3.1.9; example 1, Erg10p, ACCESSION ID NP_015297; example 2, AT5G47720, ACCESSION ID NP_001032028; example 3, AT5G48230, ACCESSION ID NP_568694), hydroxymethylglutaryl-CoA synthase (EC: 2.3.3.10; example 1, Erg13p, ACCESSION ID NP_013580; example 2, AT4G11820, ACCESSION ID NP_192919; example 3, MvaS, ACCESSION ID AAG02438), hydroxymethylglutaryl-CoA reductase (EC: 1.1.1.34; example 1, Hmg2p, ACCESSION ID NP_013555; example 2, Hmg1p, ACCESSION ID NP_013636; example 3, AT1G76490, ACCESSION ID NP_177775; example 4, AT2G17370, ACCESSION ID NP_179329, EC: 1.1.1.88, example, MvaA, ACCESSION ID P13702), and acetyl-CoA-C-acetyltransferase/hydroxymethylglutaryl-CoA reductase (EC: 2.3.1.9/1.1.1.34, example, MvaE, ACCESSION ID AAG02439). In the expression vector, the gene(s) encoding one or more enzymes involved in the mevalonate (MVA) pathway (e.g., phosphomevalonate kinase, diphosphomevalonate decarboxylase, acetyl-CoA-C-acetyltransferase/hydroxymethylglutaryl-CoA reductase (preferably, mvaE), and hydroxymethylglutaryl-CoA synthase (preferably, mvaS)) may be placed under the control of the promoter which is inversely dependent on the growth promoting agent.

In a preferred embodiment, the acetyl-CoA-C-acetyltransferase/hydroxymethylglutaryl-CoA reductase is a protein that comprises an amino acid sequence having 70% or more amino acid sequence identity to an amino acid sequence of SEQ ID NO:32, and has an acetyl-CoA-C-acetyltransferase activity or hydroxymethylglutaryl-CoA reductase activity, and the hydroxymethylglutaryl-CoA synthase is a protein that comprises an amino acid sequence having 70% or more amino acid sequence identity to an amino acid sequence of SEQ ID NO:35, and has a hydroxymethylglutaryl-CoA synthase activity. The amino acid sequence percent identity may be, for example, 75% or more, 80% or more, 85% or more, 90% or more, 95% or more, 96% or more, 97% or more, 98% or more, or 99% or more. The acetyl-CoA-C-acetyltransferase activity or hydroxymethylglutaryl-CoA reductase activity refers to an activity of producing mevalonic acid and 2 NADP and HSCoA from 3-hydroxy-3-methylglutaryl-CoA and 2 NADPH. The hydroxymethylglutaryl-CoA synthase activity refers to an activity of producing 3-hydroxy-3-methylglutaryl-CoA and HSCoA from acetoacetyl-CoA and acetyl-CoA.

The percent identity of the amino acid sequences and the percent identity of the nucleotide sequences as described later can be determined using the BLAST algorithm (Pro. Natl. Acad. Sci. USA, 90, 5873 (1993) which is incorporated herein by reference in its entirety) by Karlin and Altschul, and the FASTA algorithm (Methods Enzymol., 183, 63 (1990) which is incorporated herein by reference in its entirety) by Pearson. The programs referred to as BLASTP and BLASTN were developed based on the BLAST algorithm (see http://www.ncbi.nlm.nih.gov, which is incorporated herein by reference in its entirety). Thus, the percent identity of the nucleotide sequences and the amino acid sequences may be calculated using these programs with default settings. Also, for example, a numerical value obtained by calculating similarity as a percentage at a setting of “unit size to compare=2” using the full length of a polypeptide portion encoded in ORF with the software GENETYX Ver. 7.0.9 from Genetyx Corporation employing the Lipman-Pearson method may be used as the homology of the amino acid sequences. The lowest value among the values derived from these calculations may be employed as the percent identity of the nucleotide sequences and the amino acid sequences.

In another preferred embodiment, the acetyl-CoA-C-acetyltransferase/hydroxymethylglutaryl-CoA reductase is a protein that comprises an amino acid sequence having a mutation of one or several amino acids in the amino acid sequence of SEQ ID NO:32, and has the acetyl-CoA-C-acetyltransferase activity or hydroxymethylglutaryl-CoA reductase activity, and the hydroxymethylglutaryl-CoA synthase is a protein that comprises an amino acid sequence having a mutation of one or several amino acids in the amino acid sequence of SEQ ID NO:35, and has the hydroxymethylglutaryl-CoA synthase activity. Examples of the mutation of the amino acid residues may include deletion, substitution, addition and insertion of amino acid residues. The mutation of one or several amino acids may be introduced into one region or multiple different regions in the amino acid sequence. The term “one or several” indicates a range in which a three-dimensional structure and an activity of the protein are not impaired greatly. In the case of the protein, the number represented by “one or several” can be, for example, 1 to 100, preferably 1 to 80, more preferably 1 to 50, 1 to 30, 1 to 20, 1 to 10, or 1 to 5.

The acetyl-CoA-C-acetyltransferase/hydroxymethylglutaryl-CoA reductase preferably has an acetyl-CoA-C-acetyltransferase activity or hydroxymethylglutaryl-CoA reductase activity that is 50% or more, 60% or more, 70% or more, 80% or more, 90% or more, or 95% or more of the acetyl-CoA-C-acetyltransferase activity or hydroxymethylglutaryl-CoA reductase activity of the protein consisting of the amino acid sequence of SEQ ID NO:32 when measured under the same conditions. The hydroxymethylglutaryl-CoA synthase preferably has a hydroxymethylglutaryl-CoA synthase activity that is 50% or more, 60% or more, 70% or more, 80% or more, 90% or more, or 95% or more of the hydroxymethylglutaryl-CoA synthase activity of the protein consisting of the amino acid sequence of SEQ ID NO:35 when measured under the same conditions.

In the aforementioned enzymes, the mutation may be introduced into sites in a catalytic domain and sites other than the catalytic domain as long as an objective activity is retained. The positions of amino acid residues to be mutated which are capable of retaining the objective activity are understood by a person skilled in the art. Specifically, a person skilled in the art can recognize a correlation between structure and function, since a person skilled in the art can 1) compare the amino acid sequences of multiple proteins having the same type of activity, 2) clarify regions that are relatively conserved and regions that are not relatively conserved, and then 3) predict regions capable of playing a functionally important role and regions incapable of playing a functionally important role from the regions that are relatively conserved and the regions that are not relatively conserved, respectively. Therefore, a person skilled in the art can identify the positions of the amino acid residues to be mutated in the amino acid sequence of the aforementioned enzymes.

When the amino acid residue is mutated by substitution, the substitution of the amino acid residue may be conservative substitution. As used herein, the term “conservative substitution” refers to substitution of a certain amino acid residue with an amino acid residue having a similar side chain. Families of amino acid residues having similar side chains are well-known in the art. Examples of such families may include amino acids having a basic side chain (e.g., lysine, arginine, histidine), amino acids having an acidic side chain (e.g., aspartic acid, glutamic acid), amino acids having a non-charged polar side chain (e.g., asparagine, glutamine, serine, threonine, tyrosine, cysteine), amino acids having a non-polar side chain (e.g., glycine, alanine, valine, leucine, isoleucine, proline, phenylalanine, methionine, tryptophan), amino acids having a branched side chain at position β (e.g., threonine, valine, isoleucine), amino acids having an aromatic side chain (e.g., tyrosine, phenylalanine, tryptophan, histidine), amino acids having a hydroxyl group-containing (e.g., alcoholic, phenolic) side chain (e.g., serine, threonine, tyrosine), and amino acids having a sulfur-containing side chain (e.g., cysteine, methionine). Preferably, the conservative substitution of the amino acids may be the substitution between aspartic acid and glutamic acid, the substitution among arginine, lysine and histidine, the substitution between tryptophan and phenylalanine, the substitution between phenylalanine and valine, the substitution among leucine, isoleucine and alanine, and the substitution between glycine and alanine.

In another preferred embodiment, a gene encoding the acetyl-CoA-C-acetyltransferase/hydroxymethylglutaryl-CoA reductase may be a polynucleotide that comprises a nucleotide sequence having 70% or more nucleotide sequence identity to a nucleotide sequence of SEQ ID NO:33 or SEQ ID NO:34, and encodes a protein having the acetyl-CoA-C-acetyltransferase activity or hydroxymethylglutaryl-CoA reductase activity, and a gene encoding the hydroxymethylglutaryl-CoA synthase may be a polynucleotide that comprises a nucleotide sequence having 70% or more nucleotide sequence identity to a nucleotide sequence of SEQ ID NO:36 or SEQ ID NO:37, and encodes a protein having the hydroxymethylglutaryl-CoA synthase activity. The nucleotide sequence percent identity may be 70% or more, 75% or more, 80% or more, 85% or more, 90% or more, 95% or more, 96% or more, 97% or more, 98% or more, or 99% or more.

In another preferred embodiment, a gene encoding the acetyl-CoA-C-acetyltransferase/hydroxymethylglutaryl-CoA reductase may be a polynucleotide that hybridizes with a polynucleotide consisting of a nucleotide sequence complementary to a nucleotide sequence of SEQ ID NO:33 or SEQ ID NO:34 under a stringent condition, and encodes the protein having the acetyl-CoA-C-acetyltransferase activity or hydroxymethylglutaryl-CoA reductase activity, and a gene encoding the hydroxymethylglutaryl-CoA synthase may be a polynucleotide that hybridizes with a polynucleotide consisting of a nucleotide sequence complementary to a nucleotide sequence of SEQ ID NO:36 or SEQ ID NO:37 under a stringent condition, and encodes the protein having the hydroxymethylglutaryl-CoA synthase. The “stringent condition” refers to a condition where a so-called specific hybrid is formed whereas a non-specific hybrid is not formed. For example, such a condition is the condition where substantially the same polynucleotides having the high identity, for example, the polynucleotides having the percent identity described above hybridize to each other whereas polynucleotides having the lower identity than above do not hybridize to each other. Specifically, such a condition may include hybridization in 6×SCC (sodium chloride/sodium citrate) at about 45° C. followed by one or two or more washings in 0.2×SCC and 0.1% SDS at 50 to 65° C.

Examples of the enzymes involved in the methylerythritol phosphate (MEP) pathway may include 1-deoxy-D-xylulose-5-phosphate synthase (EC: 2.2.1.7, example 1, Dxs, ACCESSION ID NP_414954; example 2, AT3G21500, ACCESSION ID NP_566686; example 3, AT4G15560, ACCESSION ID NP_193291; example 4, AT5G11380, ACCESSION ID NP_001078570), 1-deoxy-D-xylulose-5-phosphate reductoisomerase (EC: 1.1.1.267; example 1, Dxr, ACCESSION ID NP_414715; example 2, AT5G62790, ACCESSION ID NP_001190600), 4-diphosphocytidyl-2-C-methyl-D-erythritol synthase (EC: 2.7.7.60; example 1, IspD, ACCESSION ID NP_417227; example 2, AT2G02500, ACCESSION ID NP_565286), 4-diphosphocytidyl-2-C-methyl-D-erythritol kinase (EC: 2.7.1.148; example 1, IspE, ACCESSION ID NP_415726; example 2, AT2G26930, ACCESSION ID NP_180261), 2-C-methyl-D-erythritol-2,4-cyclodiphosphate synthase (EC: 4.6.1.12; example 1, IspF, ACCESSION ID NP_417226; example 2, AT1G63970, ACCESSION ID NP_564819), 1-hydroxy-2-methyl-2-(E)-butenyl-4-diphosphate synthase (EC: 1.17.7.1; example 1, IspG, ACCESSION ID NP_417010; example 2, AT5G60600, ACCESSION ID NP_001119467), and 4-hydroxy-3-methyl-2-butenyl diphosphate reductase (EC: 1.17.1.2; example 1, IspH, ACCESSION ID NP_414570; example 2, AT4G34350, ACCESSION ID NP_567965). In the expression vector, the gene(s) encoding one or more enzymes involved in the methylerythritol phosphate (MEP) pathway may be placed under the control of the promoter which is inversely dependent on the growth promoting agent.

Further, a gene encoding the enzyme involved in the mevalonate pathway or the methylerythritol phosphate pathway that synthesizes dimethylallyl diphosphate or isopentenyl diphosphate that is a precursor of the isoprenoid compound (e.g., the substrate of the isoprene synthase) may also be introduced into the isoprene-forming microorganism. Examples of such an enzyme may include 1-deoxy-D-xylose-5-phosphate synthase that converts a pyruvate and D-glycelaldehyde-3-phosphate into 1-deoxy-D-xylose-5-phosphate, isopentenyl diphosphate isomerase that converts isopentenyl diphosphate into dimethylallyl diphosphate, and the like. In the expression vector, the gene encoding the enzyme involved in the mevalonate pathway or the methylerythritol phosphate pathway that synthesizes dimethylallyl diphosphate may be placed under the control of the constitutive promoter or inducible promoter (e.g., the promoter which is inversely dependent on the growth promoting agent).

The transformation of a host with an expression vector containing the gene(s) described above can be carried out using one or more known methods. Examples of such methods may include a competent cell method using a microbial cell treated with calcium, an electroporation method, and the like. The gene may also be introduced by infecting the microbial cell with a phage vector other than the plasmid vector.

In step 1) of the method of the present invention, the isoprenoid compound-forming microorganism is grown in the presence of the growth promoting agent at sufficient concentration. More specifically, the isoprenoid compound-forming microorganism can be grown by culturing the isoprenoid compound-forming microorganism in a culture medium in the presence of the growth promoting agent at sufficient concentration.

For example, when oxygen is used as the growth promoting agent, the isoprenoid compound-forming microorganism can be an aerobic microorganism. The aerobic microorganism can grow well in the presence of oxygen in a sufficient concentration, and thus, oxygen can act as the growth promoting agent for the aerobic microorganism. When the growth promoting agent is oxygen, a concentration of dissolved oxygen in the culture medium, which is sufficient for the growth of the aerobic microorganism in step 1), is not particularly limited as long as oxygen at that concentration can promote the growth of the aerobic microorganism, and may be, for example, 0.50 ppm or more, 1.00 ppm or more, 1.50 ppm or more, or 2.00 ppm or more. The concentration of the dissolved oxygen for the growth of the aerobic microorganism may be, for example, 7.00 ppm or less, 5.00 ppm or less, or 3.00 ppm or less.

When a phosphorus compound or an amino acid is used as the growth promoting agent, the isoprenoid compound-forming microorganism can grow well in the presence of the phosphorus compound or the amino acid in a sufficient concentration, and thus, the phosphorus compound or the amino acid can act as the growth promoting agent. When the growth promoting agent is the phosphorus compound or the amino acid, a concentration of the phosphorus compound or the amino acid that is sufficient for the growth in step 1) is not particularly limited, and may be, for example, 200 mg/L or more, 300 mg/L or more, 500 mg/L or more, 1000 mg/L or more, or 2000 mg/L or more. The concentration of the phosphorus compound or the amino acid for the growth may be, for example, 20 g/L or less, 10 g/L or less, or 5 g/L or less.

In step 2) of the method of the present invention, the formation of the isoprenoid compound by the isoprenoid compound-forming microorganism is induced by decreasing the concentration of the growth promoting agent. More specifically, the concentration of the growth promoting agent can be decreased by decreasing an amount of the growth promoting agent supplied into a culture medium. Even if the amount of the growth promoting agent supplied into the culture medium is made constant throughout steps 1) and 2), the concentration of the growth promoting agent can be decreased by utilizing the growth of the microorganism. In early phase of the growth of the microorganism in step 1), the microorganism does not grow sufficiently and the number of the microorganism in the culture medium is small. Thus, a consumption of the growth promoting agent by the microorganism is relatively low. Therefore, the concentration of the growth promoting agent in the culture medium is relatively high in the early phase of the growth. On the other hand, in the late phase of the growth of the microorganism in step 1), the microorganism grows sufficiently and the number of the microorganism is large, and thus, the consumption of the growth promoting agent by the microorganism is relatively high. Therefore, the concentration of the growth promoting agent in the culture medium becomes relatively low in the late phase of the growth. As described above, when the constant amount of the growth promoting agent continues to be supplied into the culture medium throughout steps 1) and 2), the concentration of the growth promoting agent in the culture medium is decreased in inverse proportion to the growth of the microorganism. This decreased concentration can be used as a trigger to induce the formation of the isoprenoid compound (e.g., the isoprene monomer, linalool, limonene) by the isoprenoid compound-forming microorganism.

For example, when oxygen is used as the growth promoting agent, the concentration of dissolved oxygen in the culture medium, which can induce the formation of the isoprenoid compound by the isoprenoid compound-forming microorganism can be, for example, 0.35 ppm or less, 0.15 ppm or less, or 0.05 ppm or less. The concentration of dissolved oxygen in the culture medium may be a concentration under the microaerophilic condition as described above.

Also, when a phosphorus compound or an amino acid is used as the growth promoting agent, the concentration of the phosphorus compound or the amino acid in the culture medium, which can induce the formation of the isoprenoid compound by the isoprenoid compound-forming microorganism, can be, for example, 100 mg/L or less, 50 mg/L or less, or 10 mg/L or less.

In step 3) of the method of the present invention, the isoprenoid compound is formed by culturing the isoprenoid compound-forming microorganism. More specifically, the isoprenoid compound can be formed by culturing the isoprenoid compound-forming microorganism in the culture medium under the condition of step 2) where the concentration of the growth promoting agent is decreased. The concentration of the growth promoting agent in the culture medium can be maintained at the concentration described in step 2) above in order to make the formation of the isoprenoid compound by the isoprenoid compound-forming microorganism possible. In step 3), the concentration of the isoprenoid compound formed in the culture medium can be, for example, 600 ppm or more, 700 ppm or more, 800 ppm or more, or 900 ppm or more, for example, within 6 hours, or 5, 4, or 3 hours after culturing the isoprenoid compound-forming microorganism in the culture medium under the condition of step 2).

The method of the present invention also can be characterized by an isoprenoid compound-forming rate in an induction phase, when the isoprenoid compound is the isoprene. The isoprene-forming rate (ppm/vvm/h) in the induction phase is an index value of induction efficiency that can be determined by dividing a maximum concentration of the isoprene per vvm (volume per volume per minute) by an induction time, wherein the induction time is defined as the period of time from the start of isoprene formation (the concentration of the isoprene in a fermentation gas is defined as 50 ppm) to the time point when the maximum concentration of the isoprene is achieved. The value “vvm (volume per volume per minute)” indicates ventilation amount of gas per unit time per unit culture medium in a culture apparatus. Such an isoprene-forming rate can vary depending on the type of the growth promoting agent. For example, when the oxygen is used as the growth promoting agent, such an isoprene-forming rate can be 20 ppm/vvm/h or more, 40 ppm/vvm/h or more, 50 ppm/vvm/h or more, 60 ppm/vvm/h or more, or 70 ppm/vvm/h or more, and also can be 100 ppm/vvm/h or less, 90 ppm/vvm/h or less, or 80 ppm/vvm/h or less. When the phosphorus compound is used as the growth promoting agent, such a rate can be 50 ppm/vvm/h or more, 100 ppm/vvm/h or more, 150 ppm/vvm/h or more, 200 ppm/vvm/h or more, or 250 ppm/vvm/h or more, and also can be 1000 ppm/vvm/h or less, 900 ppm/vvm/h or less, 800 ppm/vvm/h or less, 700 ppm/vvm/h or less, or 600 ppm/vvm/h or less.

In the method of the present invention, it is also possible that the period of time of culturing the isoprenoid compound-forming microorganism in step 3) is set longer than that period of step 1). In the conventional method which utilizes an inducer to obtain the isoprenoid compound in a higher amount, it is necessary to culture a microorganism for a long period of time using the inducer in the formation phase of the isoprenoid compound. However, when the cultivation is continued for a long period of time, the inducer is decomposed, and the microorganism fails to maintain the ability to produce the isoprenoid compound. Thus, it is necessary to continuously add the inducer into culture medium. As the inducer may be expensive, the cost for producing the isoprenoid compound may become inappropriate. Therefore, the culturing a microorganism for a long period of time using the inducer in the formation phase of the isoprenoid compound is problematic in that the cost for producing the isoprenoid compound can be elevated depending on the duration of the cultivation period. On the other hand, in the method of the present invention not using a particular substance such as the inducer in step 3), it is not necessary to consider the decomposition of the particular substance, and the conventional problem that the cultivation for a long period of time in the formation phase of the isoprenoid compound causes the elevation of the cost for producing the isoprenoid compound does not occur. Therefore, in the method of the present invention, the period of time of step 3) can easily be made longer, differently from the conventional method using the inducer. In the method of the present invention, the longer the period of time of step 3) is made, the more isoprenoid compound can be produced.

When the isoprenoid compound is a volatile substance, the method of the present invention can be performed in a system comprising a liquid phase and a gas phase. The volatile substance means a compound having a vapor pressure of 0.01 kPa or more at 20° C. For methods of determining a vapor pressure, static method, boiling-point method, isoteniscope, gas saturation method and DSC method are generally known (Japanese laid-open publication no. 2009-103584, which is incorporated herein by reference in its entirety). One example of the volatile isoprenoid compound includes isoprene. A closed system, for example, a reactor such as a fermentation jar or a fermentation tank, can be used as such system so as to avoid disappearance by diffusion of formed isoprene. A culture medium containing the isoprene-forming microorganism can be used as the liquid phase. The gas phase is present in the space above the liquid phase in the system, also called a headspace, and contains fermentation gas. Isoprene has the boiling point of 34° C. at standard atmosphere, it is poorly-soluble in water (solubility in water: 0.6 g/L) and it exhibits the vapor pressure of 60.8 kPa at 20° C. (e.g., Brandes et al., Physikalisch Technische Bundesanstalt (PTB), 2008, which is incorporated herein by reference in its entirety). Thus, when the isoprene-forming microorganism is cultured in the liquid phase under a temperature condition at 34° C. or higher, isoprene formed in the liquid phase can be easily transferred into the gas phase. Therefore, when such a system is used, isoprene formed in the liquid phase can be collected from the gas phase. Also, as isoprene formed in the liquid phase can be easily transferred into the gas phase, an isoprene-formation reaction by the isoprene-forming microorganism (enzymatic reaction by an isoprene synthase) in the liquid phase can also be always biased in favor of the side of isoprene formation. Limonene is poorly-soluble in water (solubility in water: 13.8 mg/L) and it exhibits the vapor pressure of 0.19 kPa at 20° C. ((R)-(+)-limonene safety data sheet; Junsei Chemical Co., Ltd.; 82205jis-1,2014/05/19). Thus, when the limonene-forming microorganism is cultured in the liquid phase under a temperature condition at 34° C. or higher, isoprene formed in the liquid phase can be easily transferred into the gas phase. Linalool is poorly-soluble in water (solubility in water: 0.16 g/100 mL) and it exhibits the vapor pressure of 0.021 Pa at 25° C. (linalool safety data sheet; Tokyo Chemical Industry Co., Ltd.; Dec. 10, 2013). Thus, when the linalool-forming microorganism is cultured in the liquid phase under a temperature condition at 34° C. or higher, isoprene formed in the liquid phase can be easily transferred into the gas phase. Therefore, when such a system is used, an isoprenoid compound formed in the liquid phase can be collected from the gas phase. Also, as a volatile isoprenoid compound formed in the liquid phase can be easily transferred into the gas phase, an isoprenoid compound-formation reaction by the isoprenoid-forming microorganism (enzymatic reaction by an isoprenoid synthetic enzyme) in the liquid phase can also be always biased in favor of the side of isoprenoid compound formation.

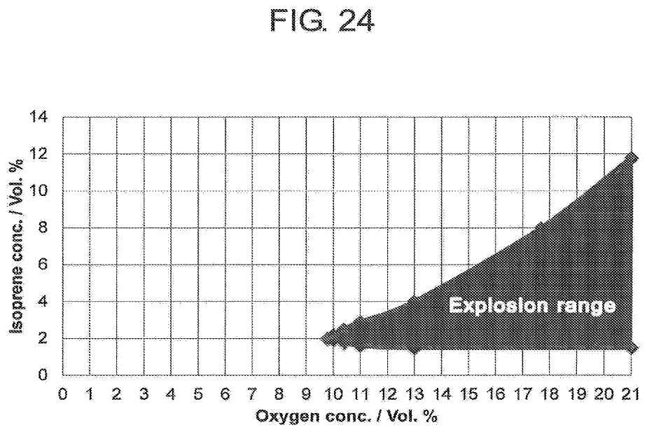

When the method of the present invention is performed in the system comprising the liquid phase and the gas phase, it is desirable to control the oxygen concentration in the gas phase. Volatile organic compounds include those having burst property. Isoprene has a burst limit of 1.0 to 9.7% (w/w) (e.g., Brandes et al., Physikalisch Technische Bundesanstalt (PTB), 2008, which is incorporated herein by reference in its entirety) and a nature to easily burst, and a burst range of isoprene varies depending on a mixing ratio of isoprene with oxygen in the gas phase (see FIG. 24, U.S. Pat. No. 8,420,360B2, which is incorporated herein by reference in its entirety). Thus, in the light of avoidance of the burst, it is necessary to control the oxygen concentration in the gas phase. Limonene has a burst range of 0.7 to 6.1% (w/w) (e.g., Brandes et al., Physikalisch Technische Bundesanstalt (PTB), 2008) and a nature to easily burst. Thus, in the light of avoidance of the burst, it is necessary to control the oxygen concentration in the gas phase.

The oxygen concentration in the gas phase can be controlled by supplying a gas in which the oxygen concentration has been regulated into the system. The gas supplied into the system may contain gas components such as nitrogen, carbon dioxide, argon, and the like other than oxygen. More specifically, the oxygen concentration in the gas phase can be controlled by adding an inert gas so that the oxygen concentration becomes equal to or less than a limit oxygen concentration in the gas having the burst range. The gas component other than oxygen is desirably the inert gas. Preferably, the gas in which the oxygen concentration has been regulated is supplied to the liquid phase, thereby indirectly controlling the oxygen concentration in the gas phase. Because, the oxygen concentration in the gas phase can be controlled by regulation of the dissolved oxygen concentration in the liquid phase as described below.

Oxygen in the gas supplied to the liquid phase is dissolved in the liquid phase, and before long reaches a saturation concentration. On the other hand, in the system in which a microorganism is present, dissolved oxygen in the liquid phase is consumed by metabolic activity of the microorganism which is cultured, and consequently the dissolved oxygen concentration decreases to a concentration less than the saturation concentration. In the liquid phase containing oxygen at concentration less than the saturation concentration, oxygen in the gas phase or oxygen in the gas freshly supplied can be transferred to the liquid phase by gas-liquid equilibrium. That is, the oxygen concentration in the gas phase decreases depending on an oxygen consumption rate by the microorganism. It is also possible to control the oxygen concentration in the gas phase by controlling the oxygen consumption rate by the microorganism which is cultured.

For example, by increasing a metabolic rate of a carbon source in the microorganism, the oxygen consumption rate can be elevated, and the oxygen concentration in the gas phase can be set to 9% (v/v) or less (e.g., 5% (v/v) or less, 0.8% (v/v) or less, 0.6% (v/v) or less, 0.5% (v/v) or less, 0.4% (v/v) or less, 0.3% (v/v) or less, 0.2% (v/v) or less, or 0.1% (v/v) or less) or substantially 0% (v/v). Alternatively, the oxygen concentration in the gas to be supplied can initially be set low. Therefore, the oxygen concentration in the gas phase can be set by considering the consumption rate of oxygen by the microorganism and the oxygen concentration in the supplied gas.

In step 1) that is the growth phase of a microorganism, when the dissolved oxygen is not present at constant concentration in the liquid phase, the microorganism cannot grow well depending on its type in the liquid phase in some cases. Also in step 1), isoprene is not formed, and thus, it is not always necessary to control the oxygen concentration in the gas phase in the light of avoidance of the burst.

For example, when oxygen is used as the growth promoting agent, the gas in which the oxygen concentration has been regulated can be supplied into the liquid phase so that the dissolved oxygen concentration that is suitable for the growth of the microorganism such as an aerobic microorganism can be maintained in the liquid phase. The gas supplied into the liquid phase can be dissolved in the liquid phase by stirring. The dissolved oxygen concentration in the liquid phase is not particularly limited as long as the isoprene-forming microorganism can grow sufficiently, and the concentration of the dissolved oxygen can vary depending on a type of the microorganism utilized such as an aerobic microorganism. For example, the concentration as described above can be employed as the concentration of dissolved oxygen in the culture medium, which is sufficient for the growth of the aerobic microorganism. Specifically, when oxygen is used as the growth promoting agent, the isoprene-forming microorganism can be grown in the presence of dissolved oxygen at the sufficient concentration as described above by supplying oxygen into a liquid phase containing the isoprene-forming microorganism in a system comprising a gas phase and the liquid phase.

In step 2) that is the induction phase of the isoprene formation, the oxygen concentration in the system is regulated in the light of avoidance of the burst by mixed gas of oxygen and isoprene which is formed in step 3) after the induction rather than in the light of growth of the microorganism in the liquid phase. For example, the oxygen concentration in the gas phase may be the same as in step 3) as described later in the light of avoidance of the burst by the mixed gas of oxygen and isoprene which is formed in the step 3).

For example, when oxygen is used as the growth promoting agent, the oxygen concentration in the system is regulated in the light of those mentioned above and in the light of inducing the formation of isoprene monomer by the isoprene-forming microorganism in the liquid phase. For example, the dissolved oxygen concentration in the liquid phase is not particularly limited as long as the aforementioned points of view are accomplished at that concentration, and varies depending on the type of the isoprene-forming microorganism utilized, the promoter to be utilized, and the like, but may be, for example, 0.35 ppm or less, 0.25 ppm or less, 0.15 ppm or less, 0.10 ppm or less, or 0.05 ppm or less. The dissolved oxygen concentration in the liquid phase may also be the concentration under the microaerophilic condition as described above. The dissolved oxygen concentration in the liquid phase can be decreased by decreasing the amount of oxygen supplied into the liquid phase. Even if the amount of oxygen supplied into the liquid phase is made constant throughout steps 1) and 2), the dissolved oxygen concentration in the liquid phase can be decreased by utilizing the growth of the microorganism which is cultured. In the early phase of the growth of the microorganism in step 1), the microorganism does not grow sufficiently and the number of the microorganism in the culture medium is relatively small. Thus, the oxygen consumption by the microorganism is relatively low. Therefore, the dissolved oxygen concentration in the liquid phase and the oxygen concentration in the gas phase are relatively high in the early phase of the growth. On the other hand, in the late phase of the growth of the microorganism, the microorganism grows sufficiently and the number of the microorganism in the culture medium is relatively large. Thus, the oxygen consumption by the microorganism is relatively high. Therefore, the dissolved oxygen concentration in the liquid phase and the oxygen concentration in the gas phase become relatively low in the late phase of the growth. As described above, when the gas containing oxygen in the constant concentration continues to be supplied into the liquid phase throughout steps 1) and 2), the dissolved oxygen concentration in the liquid phase is decreased in inverse proportion to the growth of the microorganism. This decreased oxygen concentration in the liquid phase can be used as the trigger to induce the formation of the isoprene monomer by the isoprene-forming microorganism.

In step 3) that is the formation phase of isoprene, the oxygen concentration in the gas phase is not particularly limited as long as the burst by the mixed gas of isoprene and oxygen is avoided. When the oxygen concentration in such a mixed gas is about 9.5% (v/v) or less, the burst can be avoided regardless of isoprene concentration in the gas phase (see FIG. 24). Thus, the oxygen concentration in the gas phase can be about 9.5% (v/v) or less. In the light of acquiring a safety zone of the oxygen concentration for the burst, the oxygen concentration in the gas phase can be 9% (v/v) or less, 8% (v/v) or less, 7% (v/v) or less, or 6% (v/v) or less, 5% (v/v) or less, 4% (v/v) or less, 3% (v/v) or less, 2% (v/v) or less, or 1% (v/v) or less. In step 3), the gas in which the oxygen concentration has been adjusted can be supplied into the liquid phase so that such an oxygen concentration can be maintained in the gas phase.

For example, when oxygen is used as the growth promoting agent, an isoprene monomer can be formed by culturing the isoprene-forming microorganism under the condition of the oxygen concentration in the liquid phase as described in step 2). In this case, it is desirable that the oxygen concentration in the liquid phase as described in step 2) is balanced with the oxygen concentration in the gas phase as described in step 3). The oxygen concentration in the liquid phase as described in step 2) and the oxygen concentration in the gas phase as described in step (3) can be balanced by regulating the amount of supplied gas containing oxygen while considering the type of the microorganism in the liquid phase and a degree of its growth.

When isoprene is formed in the system comprising the liquid phase and the gas phase, isoprene formed in the liquid phase can be collected from the gas phase (fermentation gas) as described above. Isoprene can be collected from the gas phase by known methods. Examples of the method of collecting isoprene from the gas phase may include an absorption method, a cooling method, a pressure swing adsorption method (PSA method), and a membrane separation method. Before being subjected to these methods, the gas phase may be subjected to a pretreatment such as dehydration, pressure elevating, pressure reducing, and the like, if necessary.

The method of the present invention may be combined with another method in terms of enhancing the amount of produced isoprenoid compound. Examples of such a method may include a method of utilizing an environmental factor such as light (Pia Lindberg, Sungsoon Park, Anastasios Melis, Metabolic Engineering 12 (2010): 70-79, which is incorporated herein by reference in its entirety) or temperature (Norma A Valdez-Cruz, Luis Caspeta, Néstor O Pérez, Octavio T Ramirez, Mauricio A Trujillo-Roldán, Microbial Cell Factories 2010, 9:1, which is incorporated herein by reference in its entirety), change of pH (EP 1 233 068 A2, which is incorporated herein by reference in its entirety), addition of surfactant (JP 11009296 A, which is incorporated herein by reference in its entirety), and auto-inducible expression system (WO 2013/151174, which is incorporated herein by reference in its entirety).

The culture medium used in the method of the present invention may contain a carbon source for forming the isoprenoid compound. The carbon source may include carbohydrates such as monosaccharides, disaccharides, oligosaccharides and polysaccharides; invert sugars obtained by hydrolyzing sucrose; glycerol; compounds having one carbon atom (hereinafter referred to as a C1 compound) such as methanol, formaldehyde, formate, carbon monoxide and carbon dioxide; oils such as corn oil, palm oil and soybean oil; acetate; animal fats; animal oils; fatty acids such as saturated fatty acids and unsaturated fatty acids; lipids; phospholipids; glycerolipids; glycerine fatty acid esters such as monoglyceride, diglyceride and triglyceride; polypeptides such as microbial proteins and plant proteins; renewable carbon sources such as hydrolyzed biomass carbon sources; yeast extracts, or combinations thereof. For a nitrogen source, inorganic ammonium salts such as ammonium sulfate, ammonium chloride and ammonium phosphate, organic nitrogen such as hydrolyzed soybeans, ammonia gas, ammonia water, and the like can be used. It is desirable that the culture medium contains required substances such as vitamin B1 and L-homoserine, or the yeast extract and the like in an appropriate amount as an organic trace nutrient source. In addition thereto, potassium phosphate, magnesium sulfate, iron ion, manganese ion, and the like are added in a small amount if necessary. The culture medium used in the present invention may be a natural medium or a synthesized medium as long as it contains the carbon source, the nitrogen source, inorganic ions, and optionally the other organic trace ingredients.

Examples of the monosaccharide may include triose such as ketotriose (dihydroxyacetone) and aldotriose (glyceraldehyde); tetrose such as ketotetrose (erythrulose) and aldotetrose (erythrose, threose); pentose such as ketopentose (ribulose, xylulose), aldopentose (ribose, arabinose, xylose, lyxose) and deoxysaccharide (deoxyribose); hexose such as ketohexose (psichose, fructose, sorbose, tagatose), aldohexose (allose, altrose, glucose, mannose, gulose, idose, galactose, talose), and deoxysaccharide (fucose, fuculose, rhamnose); and heptose such as sedoheptulose. C6 sugars such as fructose, mannose, galactose and glucose; and C5 sugars such as xylose and arabinose are preferable.

Examples of the disaccharide may include sucrose, lactose, maltose, trehalose, turanose, and cellobiose. Sucrose and lactose are preferable.

Examples of the oligosaccharide may include trisaccharides such as raffinose, melezitose and maltotriose; tetrasaccharides such as acarbose and stachyose; and other oligosaccharides such as fructooligosaccharide (FOS), galactooligosaccharide (GOS) and mannan-oligosaccharide (MOS).

Examples of the polysaccharide may include glycogen, starch (amylose, amylopectin), cellulose, dextrin, and glucan (β-1,3-glucan), and starch and cellulose are preferable.

Examples of the microbial protein may include polypeptides derived from a yeast or bacterium.

Examples of the plant protein may include polypeptides derived from soybean, corn, canola, Jatropha, palm, peanut, sunflower, coconut, mustard, cotton seed, palm kernel oil, olive, safflower, sesame and linseed.

Examples of the lipid may include substances containing one or more saturated or unsaturated fatty acids of C4 or more.

The oil can be the lipid that contains one or more saturated or unsaturated fatty acids of C4 or more and is liquid at room temperature, and examples of the oil may include lipids derived from soybean, corn, canola, Jatropha, palm, peanut, sunflower, coconut, mustard, cotton seed, Palm kernel oil, olive, safflower, sesame, linseed, oily microbial cells, Chinese tallow tree, and a combination of two or more thereof.

Examples of the fatty acid may include compounds represented by a formula RCOOH (“R” represents a hydrocarbon group having two or more carbon atoms).

The unsaturated fatty acid is a compound having at least one double bond between two carbon atoms in the group “R” as described above, and examples of the unsaturated fatty acid may include oleic acid, vaccenic acid, linoleic acid, palmitelaidic acid and arachidonic acid.

The saturated fatty acid is a compound where the “R” is a saturated aliphatic group, and examples of the saturated fatty acid may include docosanoic acid, eicosanoic acid, octadecanoic acid, hexadecanoic acid, tetradecanoic acid, and dodecanoic acid.

Among them, those containing one or more C2 to C22 fatty acids are preferable as the fatty acid, and those containing C12 fatty acid, C14 fatty acid, C16 fatty acid, C18 fatty acid, C20 fatty acid and C22 fatty acid are more preferable.

The carbon source may include salts and derivatives of these fatty acids and salts of these derivatives. Examples of the salt may include lithium salts, potassium salts, sodium salts and so forth.

Examples of the carbon source may also include combinations of carbohydrates such as glucose with lipids, oils, fats, fatty acids and glycerol fatty acid esters.

Examples of the renewable carbon source may include hydrolyzed biomass carbon sources.

Examples of the biomass carbon source may include cellulose-based substrates such as waste materials of woods, papers and pulps, leafy plants, and fruit pulps; and partial plants such as stalks, grain particles, roots and tubers.

Examples of the plant to be used as the biomass carbon source may include corn, wheat, rye, sorghum, triticale, rice, millet, barley, cassava, legume such as pea, potato, sweet potato, banana, sugar cane and tapioca.

When the renewable carbon source such as biomass is added to the culture medium, the carbon source can be pretreated. Examples of the pretreatment may include an enzymatic pretreatment, a chemical pretreatment, and a combination of the enzymatic pretreatment and the chemical pretreatment.

It is preferred that the renewable carbon source is entirely or partially hydrolyzed before being added to the culture medium.

Examples of the carbon source may also include the yeast extract and a combination of the yeast extract with the other carbon source such as glucose. The combination of the yeast extract with the C1 compound such as carbon dioxide and methanol is preferable.

In the method of the present invention, it is preferable to culture the isoprenoid compound-forming microorganism in a standard culture medium containing saline and nutrients.

The culture medium is not particularly limited, and examples of the culture medium may include ready-made general media that is commercially available such as Luria Bertani (LB) broth, Sabouraud dextrose (SD) broth, and yeast medium (YM) broth. The medium suitable for the cultivation of the specific host can be selected appropriately for the use.

It is desirable to contain appropriate minerals, salts, supplemental elements, buffers, and ingredients known for those skilled in the art to be suitable for the cultivation and to facilitate the production of the isoprenoid compound in addition to the appropriate carbon source in the cell medium.

A standard cell culture condition except that the concentration of the growth promoting agent is regulated as described above can be used as a culture condition for the isoprenoid compound-forming microorganism.

A culture temperature can be 20 to 40° C., and a pH value can be about 4.5 to about 9.5.

The isoprenoid compound-forming microorganism can be cultured under an aerobic, oxygen-free, or anaerobic condition depending on a nature of the host for the isoprene-forming microorganism. A known fermentation method such as a batch cultivation method, a feeding cultivation method or a continuous cultivation method can appropriately be used as a cultivation method.

The present invention also provides a method of producing an isoprene polymer. The method of producing the isoprene polymer according to the present invention comprises the following (I) and (II):

(I) forming an isoprene monomer by the method of the present invention; and

(II) polymerizing the isoprene monomer to form an isoprene polymer.

The step (I) can be performed in the same manner as in the method of producing the isoprene monomer according to the present invention described above. The polymerization of the isoprene monomer in the step (II) can be performed by any method known in the art (e.g., synthesis methods such as addition polymerization in organic chemistry).

### Method for Producing a Rubber Composition

The rubber composition of the present invention comprises a polymer derived from isoprene produced by the method for producing isoprene according to the present invention. The polymer derived from isoprene may be a homopolymer (i.e., isoprene polymer) or a heteropolymer comprising an isoprene monomer unit and one or more monomer units other than the isoprene monomer unit (e.g., a copolymer such as a block copolymer). Preferably, the polymer derived from isoprene is a homopolymer (i.e., isoprene polymer) produced by the method for producing isoprene polymer according to the present invention. The rubber composition of the present invention may further comprise one or more polymers other than the above polymer, one or more rubber components, and/or other components. The rubber composition of the present invention can be manufactured using the polymer derived from isoprene. For example, the rubber composition of the present invention can be prepared by mixing the polymer derived from isoprene with one or more polymers other than the above polymer, one or more rubber components, and/or other components such as a reinforcing filler, a crosslinking agent, a vulcanization accelerator and an antioxidant.

### Method for Producing a Tire

The tire of the present invention is manufactured by using the rubber composition of the present invention. The rubber composition of the present invention may be applied to any portion of the tire without limitation, which may be selected as appropriate depending on the application thereof. For example, the rubber composition of the present invention may be used in a tread, a base tread, a sidewall, a side reinforcing rubber and a bead filler of a tire. The tire can be manufactured by a conventional method. For example, a carcass layer, a belt layer, a tread layer, which are composed of un-vulcanized rubber, and other members used for the production of usual tires are successively laminated on a tire molding drum, then the drum is withdrawn to obtain a green tire. Thereafter, the green tire is heated and vulcanized in accordance with an ordinary method, to thereby obtain a desired tire (e.g., a pneumatic tire).

Other features of the invention will become apparent in the course of the following descriptions of exemplary embodiments which are given for illustration of the invention and are not intended to be limiting thereof.

### EXAMPLES

### Example 1: Construction of Isoprenoid Compound-Forming Microorganisms (Arabinose-Inducible Isoprenoid Compound-Forming Microorganism: Enterobacter aerogenes GI08-Para/ispSK Strain, and Microaerobically Inducible Isoprenoid Compound-Forming Microorganism: Enterobacter aerogenes GI08-Pbud/ispSK Strain)

1.1) Construction of G105 (ES04lld::Ptac-KDyI strain)

Enterobacter aerogenes G105 (ES04lld::Ptac-KDyI) strain was constructed by replacing a lld gene on a chromosome in ES04 strain (US2010-0297716A1) constructed from Enterobacter aerogenes AJ110637 (FERM BP-10955) strain with a Ptac-KDyI gene derived from E. coli MG1655 Ptac-KDyI strain (see Reference Example 1). A nucleotide sequence of the lld gene from Enterobacter aerogenes AJ110637 (FERM BP-10955) strain is described as SEQ ID NO:1.

1.1.1) Construction of Gene Fragment λattL-Tet^{R}-λattR-Ptac-KDyI

A Ptac-KDyI gene derived from MG1655 Ptac-KDyI strain encodes phosphomevalonate kinase (gene name: PMK) and diphosphomevalonate decarboxylase (gene name: MVD) and further isopentenyl diphosphate isomerase (gene name: yIDI) derived from Saccharomyces cerevisiae under the control of a tac promoter. PCR (TaKaRa Prime Star (registered trademark), 30 cycles of reactions at 94° C. for 10 seconds, 54° C. for 20 seconds and 72° C. for 420 seconds) with genomic DNA from MG1655 Ptac-KDyI strain as a template was carried out using primers described as SEQ ID NO:2 and SEQ ID NO:3 designed based on the above nucleotide sequence to obtain a gene fragment λattL-Tet^{R}-λattR-Ptac-KDyI having a recombinant sequence of a gene encoding D-lactate dehydrogenase at both termini (gene name: lld).

### 1.1.2) Construction of ES04/RSFRedTER Strain

ES04 strain (US 2010/0297716A1, which is incorporated herein by reference in its entirety) was cultured overnight in an LB liquid culture medium. Subsequently, 100 μL of the cultured medium was inoculated to 4 mL of a new LB liquid culture medium, and microbial cells were cultured with shaking at 34° C. for 3 hours. After collecting the microbial cells, the microbial cells were washed three times with 10% glycerol to use as competent cells. RSFRedTER was introduced by an electroporation method (Katashkina J I et al., BMC Mol Biol. 2009; 10: 34, which is incorporated herein by reference in its entirety). The electroporation was carried out using Gene Pulser II (supplied from BioRad) under the condition of an electric field intensity of 24 kV/cm, a condenser capacity of 25 μF, and a resistance value of 200Ω. The cells were cultured in an SOC culture medium (20 g/L of bacto tryptone, 5 g/L of yeast extract, 0.5 g/L of NaCl, 10 g/L of glucose) for 2 hours, and then was applied onto an LB culture medium containing 40 mg/L of chloramphenicol, and cultured for 16 hours. As a result, transformants exhibiting chloramphenicol resistance were obtained and designated as ES04/RSFRedTER strain.

1.1.3) Construction of ES04lld:: λattL-Tet^{R}-λattR-Ptac-KDyI Strain

ES04/RSFRedTER strain was cultured in an LB liquid culture medium overnight. Subsequently, 1 mL of the cultured medium was inoculated to 100 mL of an LB liquid culture medium containing IPTG and chloramphenicol at final concentrations of 1 mM and 40 mg/L, respectively, and microbial cells were cultured at 34° C. for 3 hours with shaking. After collecting the microbial cells, the microbial cells were washed three times with 10% glycerol to use as competent cells. An amplified λattL-Tet^{R}-λattR-Ptac-KDyI gene fragment purified using Wizard PCR Prep DNA Purification System (supplied from Promega) was introduced into the competent cells by the electroporation method. The cells were cultured in the SOC culture medium for 2 hours, then applied onto the LB culture medium containing 30 mg/L of tetracycline, and cultured for 16 hours. Emerging colonies were refined in the same culture medium. Subsequently, colony PCR (TaKaRa Speed Star (registered trademark), 40 cycles of reactions at 92° C. for 10 seconds, 56° C. for 10 seconds and 72° C. for 60 seconds) was carried out using primers described as SEQ ID NO:4 and SEQ ID NO:5 to identify that the lld gene on the chromosome was replaced with the λattL-Tet^{R}-λattR-Ptac-KDyI gene. The resulting colony was applied onto an LB agar medium containing 10% sucrose and 1 mM IPTG and delete the RSFRedTER plasmid to obtain ES04lld:: λattL-Tet^{R}-λattR-Ptac-KDyI strain.

1.1.4) Removal of Tetracycline Resistant Gene from ES04lld:: λattL-Tet^{R}-λattR-Ptac-KDyI Strain

In order to remove the tetracycline resistant gene from ES04lld:: λattL-Tet^{R}-λattR-Ptac-KDyI strain, an RSF-int-xis (US20100297716A1) plasmid was used. RSF-int-xis was introduced into λattL-Tet^{R}-λattR-Ptac-KDyI strain by the electroporation method, and the cells were applied onto the LB culture medium containing 40 mg/L of chloramphenicol and cultured at 30° C. to obtain λattL-Tet^{R}-λattR-Ptac-KDyI/RSF-int-xis strain. The resulting plasmid-possessing strain was refined in the LB culture medium containing 40 mg/L of chloramphenicol and 1 mM IPTG to obtain a plurality of single colonies. Subsequently, the single colony was applied onto the culture medium containing 30 mg/L of tetracycline and cultured at 37° C. overnight, and the colony was confirmed to be a strain in which the tetracycline resistant gene had been removed by confirming that the colony could not grow in this culture medium. Subsequently, the resulting strain was applied onto the LB culture medium containing 10% sucrose and 1 mM IPTG and cultured at 37° C. overnight in order to delete the RSF-int-xis plasmid from the resulting strain. A colony that exhibited chloramphenicol sensitivity among emerging colonies was designated as GI05 (ES04lld::Ptac-KDyI) strain.

1.2) Construction of GI06 (GI05 ΔpoxB::Ptac-PMK) Strain

Enterobacter aerogenes GI06 (ES04lld::Ptac-KDyIΔpoxB::Ptac-PMK) strain was constructed by replacing a pyruvate oxidase gene (gene name: poxB) on a chromosome of Enterobacter aerogenes GI05 strain with a phosphomevalonate kinase (PMK) gene derived from E. coli MG1655 Ptac-KDyI strain. A nucleotide sequence of the poxB gene from Enterobacter aerogenes AJ110637 (FERM BP-10955) strain is described as SEQ ID NO:6. A procedure will be described below.

1.2.1) Construction of Gene Fragment λattL-Km^{r}-λattR-Ptac-PMK

PCR (TaKaRa Prime Star (registered trademark), 30 cycles of reactions at 94° C. for 10 seconds, 54° C. for 20 seconds and 72° C. for 120 seconds) with genomic DNA from E. coli MG1655 Ptac-KDyI strain as the template was carried out using primers described as SEQ ID NO:7 and SEQ ID NO:8 designed based on the above nucleotide sequence to obtain a DNA fragment containing an ORF region of PMK. Also, PCR (TaKaRa Prime Star (registered trademark), 30 cycles of reactions at 94° C. for 10 seconds, 54° C. for 20 seconds and 72° C. for 90 seconds) with a DNA fragment containing λattL-Km^{r}-λattR-Ptac (WO2008090770A1) as the template was carried out using primers described as SEQ ID NO:9 and SEQ ID NO:10 to obtain a DNA fragment containing λattL-Km^{r}-λattR-Ptac. Subsequently, overlapping PCR (TaKaRa Prime Star (registered trademark), 35 cycles of reactions at 94° C. for 10 seconds, 54° C. for 20 seconds and 72° C. for 180 seconds) with the DNA fragment containing the ORF region of PMK and the DNA fragment containing λattL-Km^{r}-λattR-Ptac as the template was carried out using the primers described as SEQ ID NO:7 and SEQ ID NO:9 to obtain a gene fragment λattL-Km^{r}-λattR-Ptac-PMK having a recombinant sequence of the gene (gene name: poxB) encoding pyruvate oxidase at both termini.

### 1.2.2) Acquisition of GI06 Strain by λ-Red Method

GI05ΔpoxB:: λattL-Km^{r}-λattR-Ptac-PMK exhibiting kanamycin resistance was obtained by introducing RSFRedTER into G105 strain, introducing the λattL-Km^{r}-λattR-Ptac-PMK gene fragment into poxB by λ-Red method, and selecting in the LB culture medium containing 100 mg/L of kanamycin in the same manner as in the procedure for constructing the aforementioned G105 strain. After refining the resulting colonies in the LB culture medium, colony PCR (TaKaRa Speed Star (registered trademark), 40 cycles of reactions at 92° C. for 10 seconds, 56° C. for 10 seconds and 72° C. for 60 seconds) was carried out using primers described as SEQ ID NO:11 and SEQ ID NO:12 to confirm that the poxB gene on the chromosome was replaced with the λattL-Km^{R}-λattR-Ptac-PMK gene. Subsequently, in order to remove the kanamycin resistant gene from GI05ΔpoxB:: λattL-Km^{R}-λattR-Ptac-PMK strain from which RSFRedTER was deleted, pRSF-int-xis was introduced and the drug resistant gene was removed in the same manner as in the procedure for constructing the G105 strain. A strain exhibiting kanamycin sensitivity was designated as GI06 (GI05ΔpoxB::Ptac-PMK).

1.3) Construction of GI07 ΔpflB::Ptac-MVD Strain(GI06 ΔpflB::Ptac-MVD)

Enterobacter aerogenes GI07 (GI06 ΔpflB::Ptac-MVD) strain was constructed by replacing a pyruvate formate lyase B gene (gene name: pflB) on a chromosome from Enterobacter aerogenes GI06 strain with a diphosphomevalonate decarboxylase (MVD) gene derived from E. coli MG1655 Ptac-KDyI strain. A nucleotide sequence of the pflB gene from Enterobacter aerogenes AJ110637 (FERM BP-10955) is described as SEQ ID NO:13. The procedure will be described below.

1.3.1) Construction of Gene Fragment λattL-Km^{r}-λattR-Ptac-MVD

PCR (TaKaRa Prime Star (registered trademark), 30 cycles of reactions at 94° C. for 10 seconds, 54° C. for 20 seconds and 72° C. for 120 seconds) with genomic DNA derived from E. coli MG1655 Ptac-KDyI strain as the template was carried out using primers described as SEQ ID NO:14 and SEQ ID NO:15 designed based on the above nucleotide sequence to obtain a DNA fragment containing an ORF region of MVD. Also, PCR (TaKaRa Prime Star (registered trademark), 30 cycles of reactions at 94° C. for 10 seconds, 54° C. for 20 seconds and 72° C. for 90 seconds) with a DNA fragment containing λattL-Km^{r}-λattR-Ptac (WO 2008/090770A1, which is incorporated herein by reference in its entirety) as the template was carried out using primers described as SEQ ID NO:16 and SEQ ID NO:17 to obtain a DNA fragment containing λattL-Km^{r}-λattR-Ptac. Subsequently, overlapping PCR (TaKaRa Prime Star (registered trademark), 35 cycles of reactions at 94° C. for 10 seconds, 54° C. for 20 seconds and 72° C. for 240 seconds) with the DNA fragment containing the ORF region of MVD and the DNA fragment containing λattL-Km^{r}-λattR-Ptac as the template was carried out using the primers described as SEQ ID NO:15 and SEQ ID NO:16 to obtain a gene fragment λattL-Km^{r}-λattR-Ptac-MVD having a recombinant sequence of the gene (gene name: pflB) encoding pyruvate formate lyase B at both termini.

### 1.3.2) Acquisition of GI07 Strain by λ-Red Method

GI06ΔpflB:: λattL-Km^{r}-λattR-Ptac-MVD exhibiting kanamycin resistance was obtained by introducing RSFRedTER into GI06 strain, introducing the λattL-Km^{r}-λattR-Ptac-MVD gene fragment into pflB by the λ-Red method, and selecting in the LB culture medium containing 100 mg/L of kanamycin in the same manner as in the procedure for constructing the aforementioned GI05 strain. After refining the resulting colonies, colony PCR (TaKaRa Speed Star (registered trademark), 40 cycles of reactions at 92° C. for 10 seconds, 56° C. for 10 seconds and 72° C. for 60 seconds) was carried out using primers described as SEQ ID NO:18 and SEQ ID NO:19 to confirm that the pflB gene on the chromosome was replaced with the λattL-Km^{R}-λattR-Ptac-MVD gene. Subsequently, in order to remove the kanamycin resistant gene from GI06ΔpflB:: λattL-Km^{R}-λattR-Ptac-MVD strain from which RSFRedTER was deleted, pRSF-int-xis was introduced and the drug resistant gene was removed in the same manner as in the procedure for constructing the GI05 strain. A strain exhibiting kanamycin sensitivity was designated as GI07 (GI06ΔpflB::Ptac-MVD).

### 1.4) Construction of GI08 (GI07ΔpflA::Ptac-yIDI) Strain

Enterobacter aerogenes GI08 (GIN ΔpflA::Ptac-yIDI) strain was constructed by replacing a pyruvate formate lyase A gene (gene name: pflA) on a chromosome from Enterobacter aerogenes GI07 strain with a isopentenyl diphosphate isomerase (yIDI) gene derived from E. coli MG1655 Ptac-KDyI strain. A nucleotide sequence of the pflA gene from Enterobacter aerogenes AJ110637 (FERM BP-10955) is described as SEQ ID NO:20. The procedure will be described below.

1.4.1) Construction of Gene Fragment λattL-Km^{r}-λattR-Ptac-yIDI

PCR (TaKaRa Prime Star (registered trademark), 30 cycles of reactions at 94° C. for 10 seconds, 54° C. for 20 seconds and 72° C. for 120 seconds) with genomic DNA derived from E. coli MG1655 Ptac-KDyI strain as the template was carried out using primers described as SEQ ID NO:21 and SEQ ID NO:22 designed based on the above nucleotide sequence to obtain a DNA fragment containing an ORF region of yIDI. Also, PCR (TaKaRa Prime Star (registered trademark), 30 cycles of reactions at 94° C. for 10 seconds, 54° C. for 20 seconds and 72° C. for 90 seconds) with a DNA fragment containing λattL-Km^{r}-λattR-Ptac (WO2008090770A1) as the template was carried out using primers described as SEQ ID NO:23 and SEQ ID NO:24 to obtain a DNA fragment containing λattL-Km^{r}-λattR-Ptac. Subsequently, PCR (TaKaRa Prime Star (registered trademark), 35 cycles of reactions at 94° C. for 10 seconds, 54° C. for 20 seconds and 72° C. for 240 seconds) with the DNA fragment containing the ORF region of yIDI and the DNA fragment containing λattL-Km^{r}-λattR-Ptac as the template was carried out using the primers described as SEQ ID NO:23 and SEQ ID NO:24 to obtain a gene fragment λattL-Km^{r}-λattR-Ptac-yIDI having a recombinant sequence of the gene (gene name: pflA) encoding pyruvate formate lyase A at both termini.

### 1.4.2) Acquisition of GI08 Strain by λ-Red Method

GI07ΔpflA:: λattL-Km^{r}-λattR-Ptac-yIDI strain exhibiting kanamycin resistance was obtained by introducing RSFRedTER into GI07 strain, introducing the λattL-Km^{r}-λattR-Ptac-yIDI gene fragment into pflA by the λ-Red method, and selecting in the LB culture medium containing 100 mg/L of kanamycin in the same manner as in the procedure for constructing the aforementioned GI05 strain. After refining the resulting colonies, colony PCR (TaKaRa Speed Star (registered trademark), 40 cycles of reactions at 92° C. for 10 seconds, 56° C. for 10 seconds and 72° C. for 60 seconds) was carried out using primers described as SEQ ID NO:25 and SEQ ID NO:26 to confirm that the pflA gene on the chromosome was replaced with the λattL-Km^{R}-λattR-Ptac-yIDI gene. Subsequently, in order to remove the kanamycin resistant gene from GI07ΔpflA:: λattL-Km^{r}-λattR-Ptac-yIDI strain from which RSFRedTER was deleted, pRSF-int-xis was introduced and the drug resistant gene was removed in the same manner as in the procedure for constructing the GI05 strain. A strain exhibiting kanamycin sensitivity was designated as GI08 (GI07ΔpflA::Ptac-yIDI).

### 1.5) Construction of Arabinose-Inducible Isoprenoid Compound-Forming Microorganism GI08-Para/ispSK Strain (GI08/pMW-Para-mvaES-Ttrp/pSTV28-Ptac-ispSK)

In order to impart an ability to produce an isoprenoid compound to GI08 strain, pMW-Para-mvaES-Ttrp (see Reference Example 2) and pSTV28-Ptac-ispSK (see WO2013/179722) were introduced by the electroporation method. After preparing competent cells of GI08 strain according to the above method, pMW-Para-mvaES-Ttrp and pSTV28-Ptac-ispSK were introduced by the electroporation method, and cells were selected in the LB culture medium containing 100 mg/L of kanamycin and 60 mg/L of chloramphenicol. GI08 strain/pMW-Para-mvaES-Ttrp/pSTV28-Ptac-ispSK retaining both the plasmids was designated as GI08-Para/ispSK strain.

1.6) Construction of pMW-Pbud-mvaES

It has been already known that Enterobacter aerogenes forms 2,3-butandiol under a microaerophilic condition (Converti, A et al., Biotechnol. Bioeng., 82, 370-377, 2003). An enzyme group involved in a formation pathway and a catalytic reaction of 2,3-butandiol has been already elucidated, and their gene information and amino acid sequences have been demonstrated from the genome sequence (NC_015663) of Enterobacter aerogenes KCT2190. The formation pathway of 2,3-butandiol is composed of α-acetolactate decarboxylase (gene name: budA), acetolactate synthase (gene name: budB), and further acetoin reductase (gene name budC). These genes form an operon on the genome sequence, and its expression amount is controlled by BudR (gene name: budR) that is a transcription factor. A promoter region for BudR and the bud operon was cloned into pMW-Para-mvaES-Ttrp by the following procedure. The promoter region for BudR and the bud operon is shown as SEQ ID NO:29.

PCR (TaKaRa Prime Star (registered trademark), 30 cycles of reactions at 94° C. for 10 seconds, 54° C. for 20 seconds and 72° C. for 120 seconds) with genomic DNA derived from Enterobacter aerogenes AJ11063 strain as the template was carried out using primers described as SEQ ID NO:27 and SEQ ID NO:28 to obtain a DNA fragment containing an ORF region of BudR and the promoter region for the bud operon.

Subsequently, PCR (TaKaRa Prime Star (registered trademark), 30 cycles of reactions at 94° C. for 10 seconds, 54° C. for 20 seconds and 72° C. for 240 seconds) with pMW-Para-mvaES-Ttrp as the template was carried out using primers described as SEQ ID NO:30 and SEQ ID NO:31 to obtain a DNA fragment of pMW-Para-mvaES-Ttrp in which an arabinose promoter had been deleted. The DNA fragment containing the ORF region of BudR and the promoter region of the bud operon was ligated to the DNA fragment of pMW-Para-mvaES-Ttrp in which the arabinose promoter had been deleted using In-Fusion HD Cloning Kit (supplied from Clontech). The resulting plasmid in which the arabinose promoter had been replaced with the ORF region of BudR and the promoter region for the bud operon was designated as pMW-Pbud-mvaES-Ttrp.

### 1.7) Construction of Microaerobically Inducible Isoprenoid Compound-Forming Microorganism GI08-Pbud/ispSK Strain (GI08/pMW-Pbud-mvaES/pSTV28-Ptac-ispSK)

In order to impart the ability to produce an isoprenoid compound to GI08 strain, pMW-Pbud-mvaES-Ttrp and pSTV28-Ptac-ispSK (see WO2013/179722) were introduced by the electroporation method. After preparing competent cells of GI08 strain according to the above method, pMW-Pbud-mvaES-Ttrp and pSTV28-Ptac-ispSK were introduced by the electroporation method, and cells were selected in the LB culture medium containing 100 mg/L of kanamycin and 60 mg/L of chloramphenicol. GI08 strain/pMW-Pbud-mvaES-Ttrp/pSTV28-Ptac-ispSK retaining both the plasmids was designated as GI08-Pbud/ispSK strain.

### Example 2: Condition for Jar Culture of Isoprenoid Compound-Forming Microorganisms, GI08-Para/ispSK Strain and GI08-Pbud/ispSK Strain

A jar culture was carried out for growing microbial cells of the isoprenoid compound-forming microorganisms, GI08-Para/ispSK strain and GI08-Pbud/ispSK strain. A fermentation jar (system comprising a liquid phase and a gas phase) having a 1 L volume was used for the jar culture. A glucose medium was prepared in a composition shown in Table 1. Microbial cells of the isoprenoid compound-forming microorganisms, GI08-Para/ispSK strain and GI08-Pbud/ispSK strain were applied onto an LB plate containing chloramphenicol (60 mg/L) and kanamycin (50 mg/L), and cultured at 37° C. for 16 hours. After adding 0.3 L of the glucose culture medium into the fermentation jar having the 1 L volume, and microbial cells sufficiently grown on one plate were inoculated thereto, and the culture was started. The culture was carried out under a condition at pH 7.0 (controlled by ammonia gas) at 30° C. and air (oxygen concentration: 20% (v/v)) was supplied at 150 mL/minute into the culture medium. Dissolved oxygen (DO) in the culture medium was measured using a galvanic mode DO sensor SDOU model (supplied from ABLE & Biott Co., Ltd), and controlled by stirring so that DO was a given concentration. During the cultivation, a solution of glucose adjusted to 500 g/L was continuously added so that a glucose concentration in the culture medium was 10 g/L or more. An OD value (indicator for growth of microorganism) was measured at 600 nm using a spectrophotometer (HITACHI U-2900).

The detection limit of the galvanic mode DO sensor SDOU model used for the measurement of the DO concentration is 0.003 ppm. Hereinafter, when the measured DO concentration is below the detection limit, it is represented by “DO≈0 ppm”.

TABLE 1
Composition of glucose medium

| | Final concentration | |
|---|---|---|
| Group A | | |
| Glucose | 80 | g/L |
| MgSO₄•7aq | 2.0 | g/L |
| Group B | | |
| (NH₄)₂SO₄ | 2.0 | g/L |
| KH₂PO₄ | 2.0 | g/L |
| FeSO₄•7aq | 20 | mg/L |
| MnSO₄•5aq | 20 | mg/L |
| Yeast Extract | 4.0 | g/L |

Each 0.15 L of Group A and Group A was prepared, and then sterilized with heat at 115° C. for 10 minutes. After cooling, Group A and Group B were mixed, and chloramphenicol (60 mg/L) and kanamycin (50 mg/L) were added, and used as the medium.

### Example 3: Production of Isoprenoid Compound by Isoprenoid Compound-Forming Microorganism

### 3.1) Induction to Formation Phase of Isoprene

In an arabinose-inducible isoprenoid compound-forming microorganism (GI08-Para/ispSK), genes upstream of the mevalonate pathway are expressed by an arabinose inducible promoter, and thus an amount of isoprene produced in the presence of L-arabinose (Wako Pure Chemical Industries, Ltd.) is notably enhanced. To induce to a formation phase of isoprene, L-arabinose was added at a final concentration of 20 mM when the OD value by analysis of the culture medium with time was 16.

In a microaerobically inducible isoprenoid compound-forming microorganism (GI08-Pbud/ispSK), genes upstream of the mevalonate pathway are expressed and controlled by the promoter for the bud operon, which is a microaerobically inducible promoter, and thus an amount of isoprene produced under a microaerophilic condition is notably enhanced. In this Example, the formation phase of isoprene was induced by culturing under a constant condition for a ventilated amount and a stirring frequency and making the dissolved oxygen concentration in the culture medium to be the detection limit (DO≈0 ppm) or below with the increase of the microbial cells.

After inducing the isoprene formation by the isoprenoid compound-forming microorganism as described above, the cultivation of the isoprenoid compound-forming microorganism was continued for the isoprene formation.

### 3.2) Measurement of Isoprene Concentration in Fermentation Gas

Isoprene is poorly soluble in water and is easily volatilized. Thus, isoprene formed in the liquid phase (culture medium) is rapidly transferred as a fermentation gas into the gas phase. Therefore, the formed isoprene was measured by quantifying an isoprene concentration in the fermentation gas. Specifically, the fermentation gas was collected in a gas bag on a timely basis after inducing the isoprene formation, and the isoprene concentration was quantified using gas chromatography (GC-2010 Plus AF supplied from Shimadzu Corporation). A standard curve for isoprene was made using the following isoprene standard samples. An analysis condition for the gas chromatography will be described below.

### Preparation of Isoprene Standard Samples

A reagent isoprene (supplied from Tokyo Chemical Industry, specific gravity: 0.681) was diluted with cooled methanol to 10, 100, 1,000, 10,000 and 100,000 times to prepare standard solutions for addition. Subsequently, each 1 μL of each standard solution for the addition was added to a headspace vial in which 1 mL of water had been already added, and used as a standard sample.

Headspace sampler (Turbo Matrix 40 supplied from Perkin Elmer)
Heat retention temperature for vial: 40° C.
Heat retention time for vial: 30 minutes
Pressurization time: 3.0 minutes
Injection time: 0.02 minutes
Needle temperature: 70° C.
Transfer temperature: 80° C.
Carrier gas pressure (high purity helium): 124 kPa
Gas chromatography (GC-2010 Plus AF, supplied from Shimadzu Corporation)
Column: Rxi (registered trade name) −1 ms: length 30 m, inner diameter 0.53 mm, liquid phase membrane thickness 1.5 μm, cat #13370)
Column temperature: 37° C.

### Pressure: 24.8 kPa

Column flow rate: 5 mL/minute
Inflow method; Split 1:0 (actual measurement 1:18)
Transfer flow amount: 90 mL
GC injection amount: 1.8 mL (transfer flow amount×injection time)
Sample amount injected into column: 0.1 mL
Inlet temperature 250° C.
Detector: FID (hydrogen 40 mL/minute, Air 400 mL/minute, makeup gas helium 30 mL/minute)
Detector temperature: 250° C.

### 3.3) Isoprene Formation by Culturing Arabinose-Inducible Isoprenoid Compound-Forming Microorganism and Microaerobically Inducible Isoprenoid Compound-Forming Microorganism

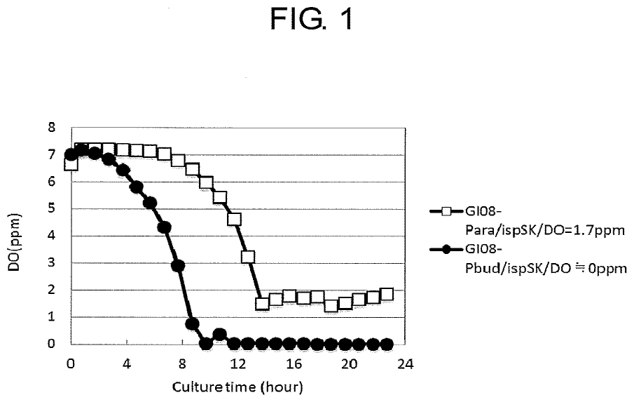

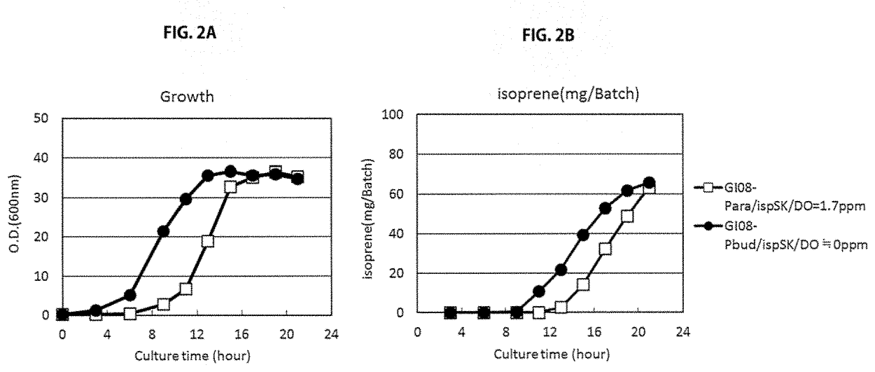

The arabinose-inducible isoprenoid compound-forming microorganism (GI08-Para/ispSK) and microaerobically inducible isoprenoid compound-forming microorganism (GI08-Pbud/ispSK) were cultured under the jar culture condition described in above Example 2, and amounts of formed isoprene were measured. As shown in FIG. 1, by controlling the stirring frequency during the cultivation, the dissolved oxygen concentration in the culture medium in which GI08-Para/ispSK was cultured was kept at 1.7 ppm from 14 hours after the start of the cultivation, and the dissolved oxygen concentration in the culture medium in which GI08-Pbud/ispSK was cultured became the detection limit or below (DO≈0 ppm) from 9 hours after the start of the cultivation. As shown in FIG. 2A, GI08-Para/ispSK and GI08-Pbud/ispSK grew well under the jar culture condition using the fermentation jar (system comprising a liquid phase and a gas phase). As shown in FIG. 2B, the production of isoprene was detected at 11 hours and 8 hours after the start of the cultivation in GI08-Pbud/ispSK and GI08-Para/ispSK, respectively, indicating that the production of isoprene was induced. The amounts of formed isoprene until 21 hours after starting the cultivation were 63 mg and 66 mg in GI08-Para/ispSK and GI08-Pbud/ispSK, respectively. This result indicates that the arabinose-inducible isoprenoid compound-forming microorganism and the microaerobically inducible isoprenoid compound-forming microorganism have an equivalent ability to produce isoprene.

### Example 4: Amount of Isoprene Formed by Microaerobically Inducible Isoprenoid Compound-Forming Microorganism Under Various Dissolved Oxygen Condition

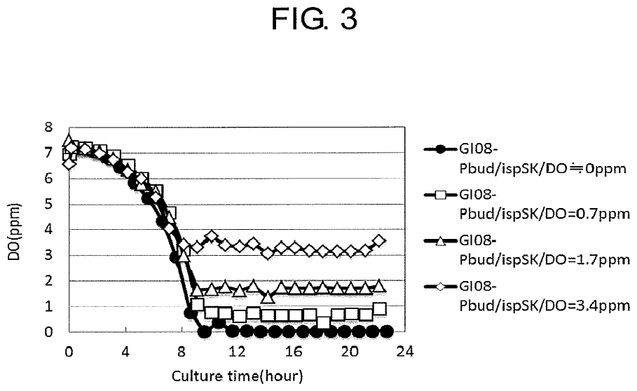

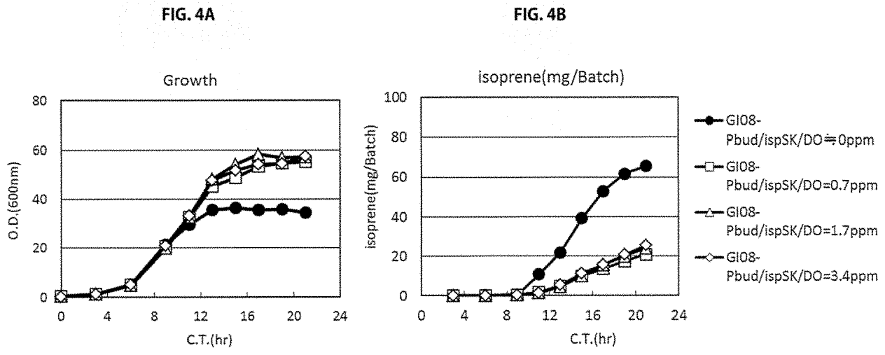

The microaerobically inducible isoprenoid compound-forming microorganism was cultured under the condition of the dissolved oxygen at DO≈0 ppm, DO=0.7 ppm, DO=1.7 ppm and DO=3.4 ppm by supplying air containing 20% oxygen and controlling the stirring frequency during the cultivation. Changes with time of the dissolved oxygen concentration in the culture medium are shown in FIG. 3. The OD values (indicator for the growth of the microorganism) in the culture medium were 35, 55, 57 and 57 under the condition of DO≈0 ppm, DO=0.7 ppm, DO=1.7 ppm and DO=3.4 ppm, respectively. The OD value was higher under the aerobic culture condition than that under the condition of DO≈0 ppm (FIGS. 4A and 4B). Under the condition of DO≈0 ppm, the production of isoprene was induced after 9 hours after starting the cultivation at which DO became DO≈0 ppm, and high productivity of isoprene was observed after 13 hours after starting the cultivation at which the OD value became plateau. The amounts of isoprene formed until 21 hours after starting the cultivation were 66 mg, 21 mg, 24 mg and 25 mg under the condition of DO≈0 ppm, DO=0.7 ppm, DO=1.7 ppm and DO=3.4 ppm, respectively (FIGS. 4A and 4B). This result indicated that the dissolved oxygen concentration became the detection limit or below, thereby transferring to the formation phase of isoprene, and subsequently the production of isoprene was also continued in the microaerobically inducible isoprenoid compound-forming microorganism.

### Example 5: Construction of Microaerobically Inducible Isoprenoid Compound-Forming Microorganism (SWITCH-Plld/IspSM), Phosphate Deficiency-Inducible Isoprenoid Compound-Forming Microorganism (SWITCH-PphoC/IspSM, SWITCH-PpstS/IspSM) and Arabinose-Inducible Isoprenoid Compound-Forming Microorganism (SWITCH-Para/IspSM)

5-1) Construction of pMW-Para-mvaES-Ttrp
5-1-1) Chemical Synthesis of mvaES Gene Derived from Enterococcus faecalis

A nucleotide sequence and an amino acid sequence of mvaE encoding acetyl-CoA acetyltransferase and hydroxymethylglutaryl-CoA reductase and derived from Enterococcus faecalis have been already known (Accession number of nucleotide sequence: AF290092.1(1479 . . . 3890), Accession number of amino acid sequence: AAG02439) (J. Bacteriol. 182 (15), 4319-4327 (2000)). The amino acid sequence of the mvaE protein derived from Enterococcus faecalis and the nucleotide sequence of its gene are shown as SEQ ID NO:32 and SEQ ID NO:33, respectively. In order to efficiently express the mvaE gene in E. coli, an mvaE gene in which codon usage in E. coli had been optimized was designed, and this was designated as EFmvaE. This nucleotide sequence is shown as SEQ ID NO:34. The mvaE gene was chemically synthesized, then was cloned into pUC57 (supplied from GenScript), and the resulting plasmid was designated as pUC57-EFmvaE.

5-1-2) Chemical Synthesis of mvaS Gene Derived from Enterococcus faecalis

A nucleotide sequence encoding hydroxymethylglutaryl-CoA synthase and derived from Enterococcus faecalis, and its amino acid sequence have been already known (Accession number of nucleotide sequence: AF290092.1, complement (142 . . . 1293), Accession number of amino acid sequence: AAG02438) (J. Bacteriol. 182(15), 4319-4327 (2000), which is incorporated herein by reference in its entirety). The amino acid sequence of the mvaS protein derived from Enterococcus faecalis and the nucleotide sequence of the mvaS gene are shown as SEQ ID NO:35 and SEQ ID NO:36, respectively. In order to efficiently express the mvaS gene in E. coli, an mvaS gene in which the codon usage in E. coli had been optimized was designed, and this was designated as EFmvaS. This nucleotide sequence is shown as SEQ ID NO:37. The mvaS gene was chemically synthesized, then was cloned into pUC57 (supplied from GenScript), and the resulting plasmid was designated as pUC57-EFmvaS.

5-1-3) Construction of Expression Vector for Arabinose-Inducible mvaES

An expression vector for arabinose-inducible gene upstream of the mevalonate pathway was constructed by the following procedure. PCR with plasmid pKD46 as the template was carried out using synthesized oligonucleotides shown as SEQ ID NO:38 and SEQ ID NO:39 as primers to obtain a PCR fragment containing Para composed of araC and an araBAD promoter derived from E. coli. PCR with plasmid pUC57-EFmvaE as the template was carried out using synthesized oligonucleotides shown as SEQ ID NO:40 and SEQ ID NO:41 as primers to obtain a PCR fragment containing the EFmvaE gene. PCR with plasmid pUC57-EFmvaS as the template was carried out using synthesized oligonucleotides shown as SEQ ID NO:42 and SEQ ID NO:43 as primers to obtain a PCR fragment containing the EFmvaS gene. PCR with plasmid pSTV-Ptac-Ttrp (WO2013069634A1) as the template was carried out using synthesized oligonucleotides shown as SEQ ID NO:44 and SEQ ID NO:45 as primers to obtain a PCR fragment containing a Ttrp sequence. Prime Star polymerase (supplied from Takara Bio Inc.) was used for PCR for obtaining these four PCR fragments. A reaction solution was prepared according to a composition attached to a kit, and DNA was amplified through 30 cycles of reactions at 98° C. for 10 seconds, 55° C. for 5 seconds and 72° C. for one minute per kb. PCR with the purified PCR product containing Para and PCR product containing the EFmvaE gene as the template was carried out using synthesized oligonucleotides shown as SEQ ID NO:38 and SEQ ID NO:41 as primers, and PCR with the purified PCR product containing the EFmvaS gene and PCR product containing Ttrp as the template was carried out using synthesized oligonucleotides shown in SEQ ID NO:42 and SEQ ID NO:45 as primers. As a result, a PCR product containing Para and the EFmvaE gene and a PCR product containing the EFmvaS gene and Ttrp were obtained. A plasmid pMW219 (supplied from Nippon Gene Co., Ltd.) was digested with SmaI according to a standard method. pMW219 after being digested with SmaI was ligated to the purified PCR product containing Para and the EFmvaE gene and the purified PCR product containing the EFmvaS gene and Ttrp using In-Fusion HD Cloning Kit (supplied from Clontech). The resulting plasmid was designated as pMW-Para-mvaES-Ttrp.

### 5-2) Construction of the Integrative Conditionally Replicated Plasmids Carrying Genes of Upper and Lower Mevalonate Pathways

To construct the integrative plasmids carrying genes of upper and lower mevalonate pathways the pAH162-λattL-TcR-λattR vector (Minaeva N I et al., BMC Biotechnol., 2008; 8: 63, which is incorporated herein by reference in its entirety) was used.

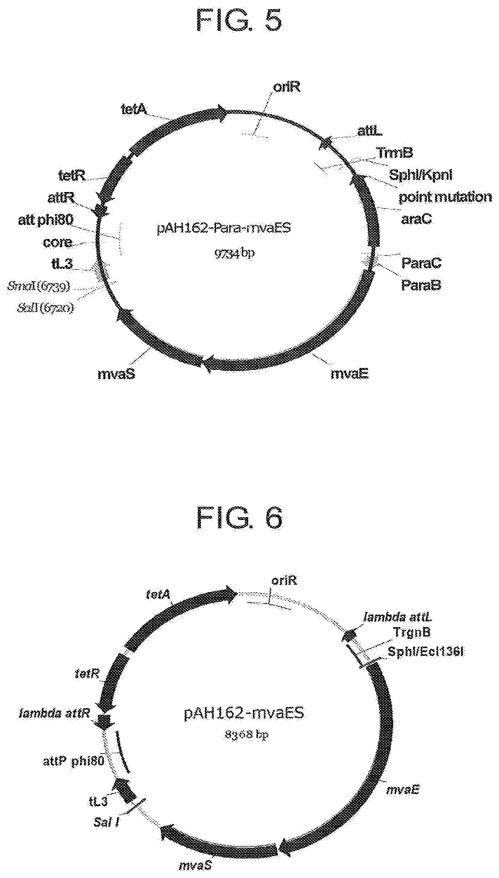

KpnI-SalI fragment of pMW-Para-mvaES-Ttrp was cloned into SphI-SalI recognition sites of pAH162-λattL-TcR-λattR. As a result, the pAH162-Para-mvaES plasmid carrying mvaES operon from E. faecalis under control of the E. coli Para promoter and repressor gene araC have been constructed (FIG. 5).

In order to obtain a variant of promoter-deficient operon, an Ecl136II-SalI fragment of pMW219-Para-mvaES-Ttrp was subcloned into the same integrative vector. A map of the resulting plasmid is shown in FIG. 6.

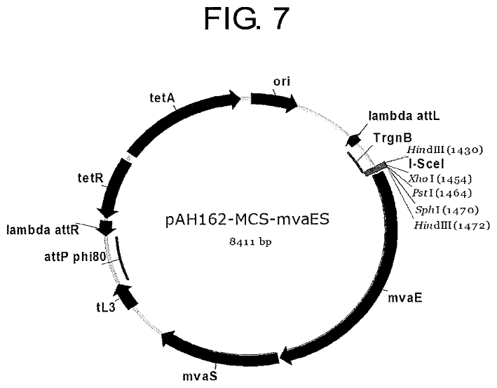

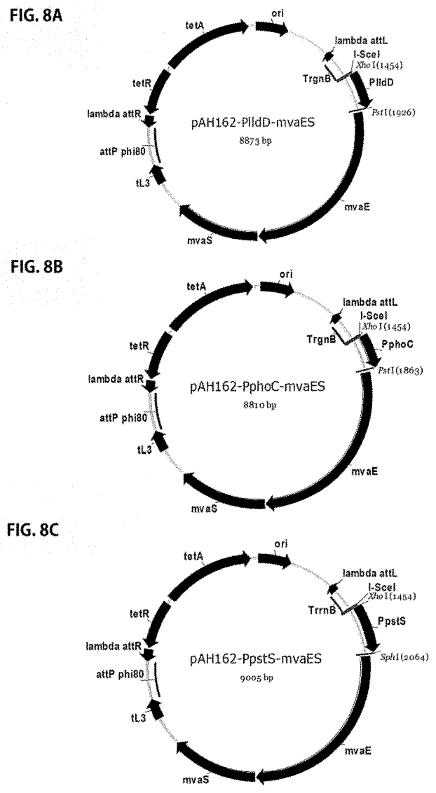

A set of plasmids for chromosome fixation, which retained the mvaES gene under the control of a different promoter was constructed. For this purpose, a polylinker containing I-SceI, XhoI, PstI and SphI recognition sites was inserted into unique HindIII recognition site located upstream of the mvaES gene. In order to accomplish this purpose, annealing was carried out using the primers 1 and 2 (Table 2). After that the resulting double-stranded DNA fragment was 5′ phosphorylated with polynucleotide kinase and the resulting phosphorylated fragment was inserted into a pAH162-mvaES plasmid cleaved with HindIII by a ligation reaction. The resulting pAH162-MCS-mvaES plasmid (FIG. 7) is convenient for cloning of a promoter while a desired orientation is kept before the mvaES gene. DNA fragments retaining a regulatory region of a lldD, phoC and pstS genes were formed by PCR with genomic DNA from P. ananatis SC17(0) strain (Katashkina J I et al., BMC Mol Biol., 2009; 10: 34) as the template using primers 3 and 4, primers 5 and 6, and primers 7 and 8 (Table 2), respectively, and cloned into an appropriate restriction enzyme recognition site of pAH162-MCS-mvaES. The resulting plasmids are shown in FIGS. 8A, 8B, and 8C. The cloned promoter fragments were sequenced and confirmed to exactly correspond to predicted nucleotide sequences.

5-2-2) Construction of pAH162-Km-Ptac-KDyI Plasmid for Chromosome Fixation

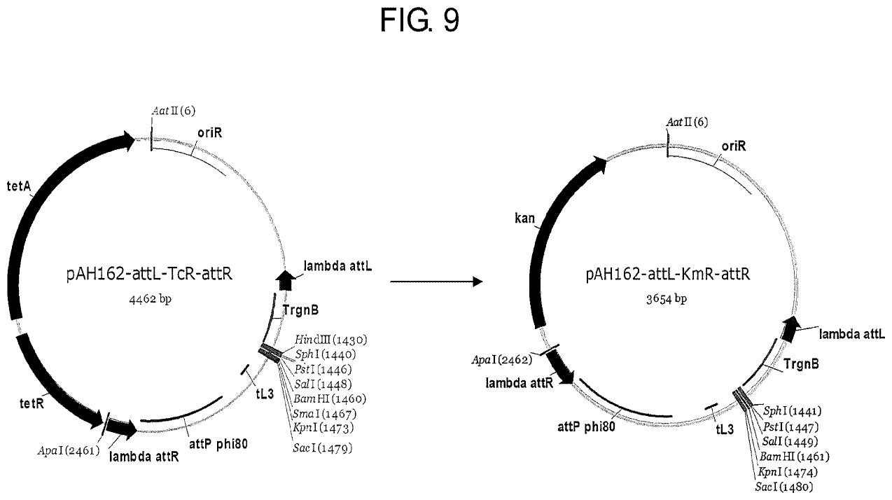

An AatII-ApaI fragment of pAH162-λattL-Tc^{R}-λattR containing a tetAR gene (Minaeva N I et al., BMC Biotechnol., 2008; 8: 63, which is incorporated herein by reference in its entirety) was replaced with a DNA fragment obtained by PCR with a pUC4K plasmid (Taylor L A and Rose R E., Nucleic Acids Res., 16, 358, 1988, which is incorporated herein by reference in its entirety) as the template using the primers 9 and 10 (Table 2). As a result, pAH162-λattL-Km^{R}-λattR was obtained (FIG. 9).

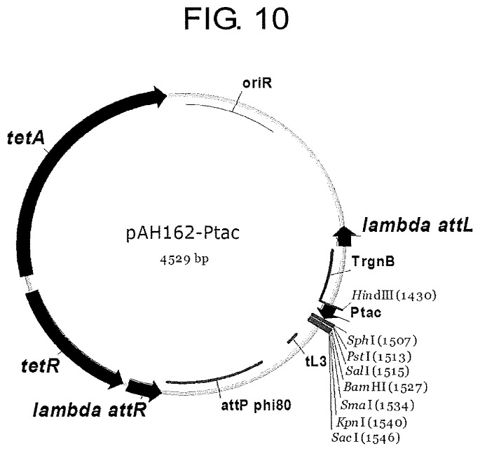

A P_{tac} promoter was inserted into a HindIII-SphI recognition site of the pAH162-λattL-Tc^{R}-λattR vector (Minaeva N I et al., BMC Biotechnol., 2008; 8: 63, which is incorporated herein by reference in its entirety). As a result, an expression vector pAH162-P_{tac} for the chromosome fixation was constructed. The cloned promoter fragment was sequenced and confirmed to be the sequence as designed. A map of pAH162-P_{tac} is shown in FIG. 10.

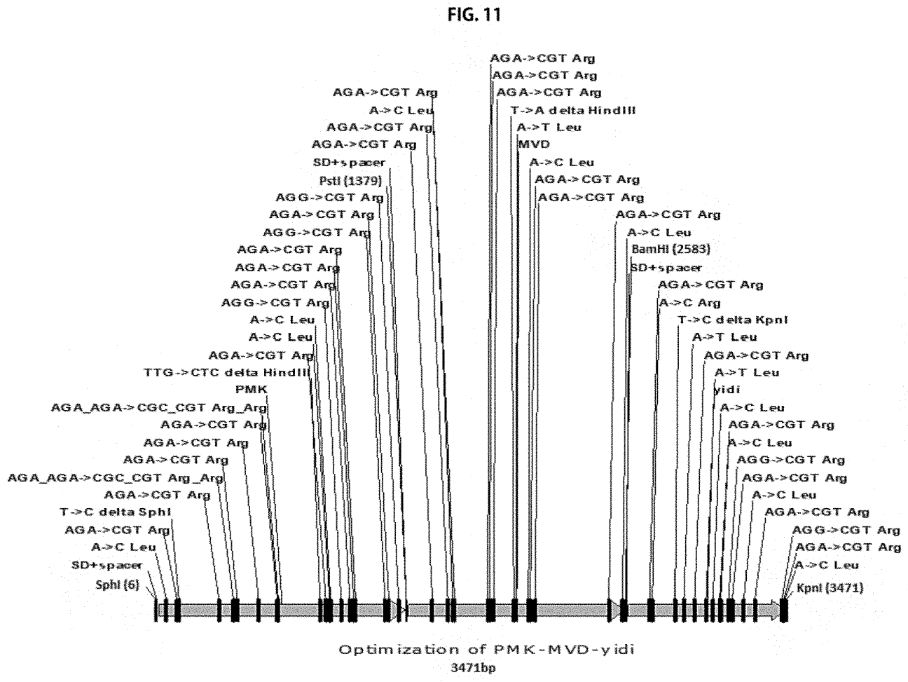

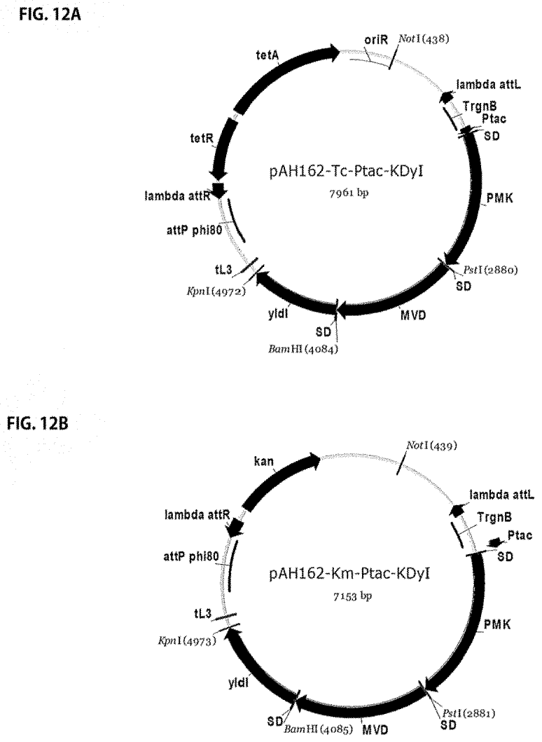

A DNA fragment that retained a PMK, MVD and yldI genes derived from S. cerevisiae, in which rare codons had been replaced with synonymous codons, and had been synthesized by ATG Service Gene (Russia) (FIG. 11) was subcloned into a SphI-KpnI restriction enzyme recognition site of the vector pAH162-Ptac for the chromosome fixation. The DNA sequence including synthesized KDyI operon is shown in SEQ ID NO:70. The resulting plasmid pAH162-Tc-Ptac-KDyI retaining a Ptac-KDyI expression cassette is shown in FIG. 12A. Subsequently, for the purpose of replacing a drug resistant marker gene, a NotI-KpnI fragment of pAH162-Tc-P_{tac}-KDyI retaining the tetAR gene was replaced with a corresponding fragment of pAH162-λattL-Km^{R}-λattR. As a result, a plasmid pAH162-Km-Ptac-KDyI having a kanamycin resistant gene, kan as a marker was obtained (FIG. 12B).

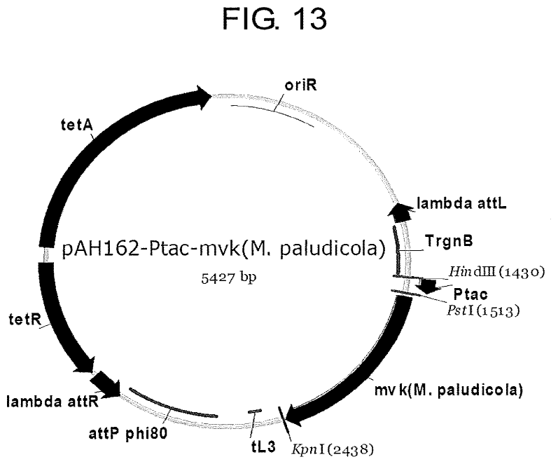

A chemically synthesized DNA fragment containing a coding region of a putative mvk gene derived from SANAE (for full-length genomic sequence, see GenBank Accession Number AP011532) that was strain of Methanocella paludicola, which had been ligated to a classical SD sequence, was cloned into a PstI-KpnI recognition site of the above integrative expression vector pAH162-P_{tac}. A map of the plasmid for the chromosome fixation retaining the mvk gene is shown in FIG. 13.

5-3) Construction of Recipient Strain SC17(0) ΔampC::attBₚₕᵢ₈₀ ΔampH::attBₚₕᵢ₈₀ ΔCrt::P_{tac}-Mvk (M. paludicola)

Using two-stage technique including λ-Red dependent integration of a PCR amplified DNA fragment containing the kan gene flanked by attLₚₕᵢ₈₀ and attRₚₕᵢ₈₀ and 40 bp sequences homologous to a target chromosome site (Katashkina J I et al., BMC Mol Biol., 2009; 10: 34, which is incorporated herein by reference in its entirety), and subsequent phi80 Int/Xis dependent removal of the kanamycin resistant marker (Andreeva I G et al., FEMS Microbiol Lett., 2011; 318(1): 55-60, which is incorporated herein by reference in its entirety), chromosomal modifications of ΔampH::attBₚₕᵢ₈₀ and ΔampC::attBₚₕᵢ₈₀ was introduced into P. ananatis SC17(0) strain in a stepwise fashion. SC17(0) is λ-Red resistant derivative of P. ananatis AJ13355 (Katashkina J I et al., BMC Mol Biol., 2009; 10: 34, which is incorporated herein by reference in its entirety); an annotated full-length genomic sequence of P. ananatis AJ13355 is available as PRJDA162073 or GenBank Accession Numbers AP012032.1 and AP012033.1. Using pMWattphi plasmid (Minaeva N I et al., BMC Biotechnol., 2008; 8:63, which is incorporated herein by reference in its entirety) as the template and using primers 11 and 12, and primers 13 and 14 (Table 2) as the primers, DNA fragments used for integration into an ampH and ampC gene regions, respectively, were formed. The primers 15 and 16, and the primers 17 and 18 (Table 2) were used to verify the resulting chromosome modifications by PCR.

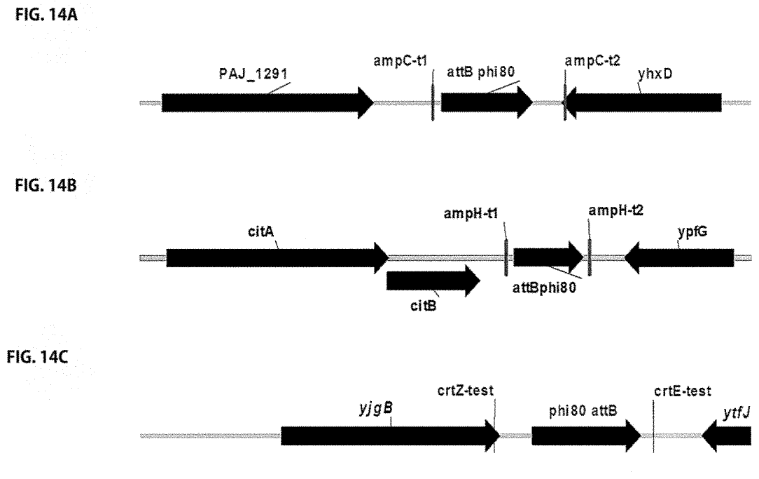

In parallel, a derivative of P. ananatis SC17(0) retaining an attB site of phi80 phage in place of a crt operon located on pEA320 320 kb megaplasmid that was a part of P. ananatis AJ13355 genome was constructed. In order to obtain this strain, λ-Red dependent integration of PCR-amplified DNA fragment retaining attLₚₕᵢ₈₀-kan-attRₚₕᵢ₈₀ flanked by a 40 bp region homologous to a target site in genome was carried out according to the previously described technique (Katashkina J I et al., BMC Mol Biol., 2009; 10: 34). Therefore, a DNA fragment to be used in the replacement of the crt operon with attLₚₕᵢ₈₀-kan-attRₚₕᵢ₈₀ was amplified in the reaction using the primers 19 and 20 (Table 2). A pMWattphi plasmid (Minaeva N I et al., BMC Biotechnol., 2008; 8: 63, which is incorporated herein by reference in its entirety) was used as template in this reaction. The resulting integrated product was designated as SC17(0) Δcrt::attLₚₕᵢ₈₀-kan-attRₚₕᵢ₈₀. The primers 21 and 22 (Table 2) were used to verify the chromosome structure of SC17(0) Δcrt::attLₚₕᵢ₈₀-kan-attRₚₕᵢ₈₀ by PCR. The kanamycin resistance marker was removed from the constructed strain according to the reported technique using a pAH129-cat helper plasmid (Andreeva I G et al., FEMS Microbiol Lett., 2011; 318(1): 55-60, which is incorporated herein by reference in its entirety). The Oligonucleotides 21 and 22 were used to verify the resulting SC17(0) Δcrt::attBₚₕᵢ₈₀ strain by PCR. Maps of genome-modified products, ΔampC::attBₚₕᵢ₈₀, ΔampH::attBₚₕᵢ₈₀ and Δcrt::attBₚₕᵢ₈₀ are shown in FIGS. 14A, 14B, and 14C, respectively.

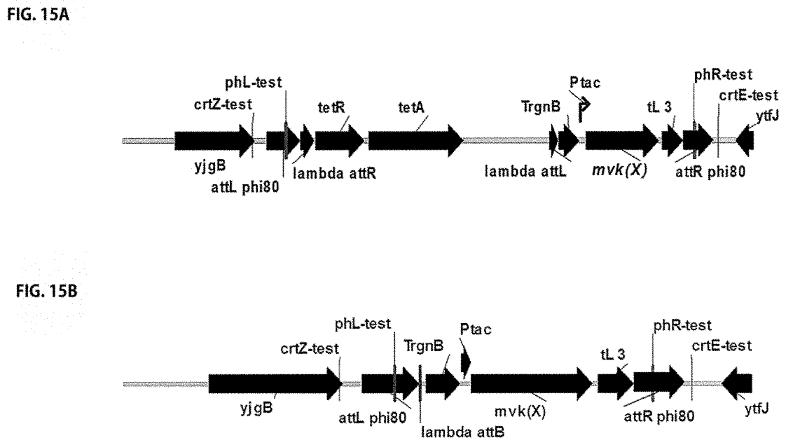

The aforementioned pAH162-Ptac-mvk (M. paludicola) plasmid was integrated into an attBₚₕᵢ₈₀ site of SC17(0) Δcrt::attBₚₕᵢ₈₀ according to the reported protocol (Andreeva I G et al., FEMS Microbiol Lett., 2011; 318(1): 55-60, which is incorporated herein by reference in its entirety). The integration of the plasmid was confirmed by the polymerase chain reaction using the primers 21 and 23 and the primers 22 and 24 (Table 2). As a result, SC17(0) Δcrt::pAH162-P_{tac}-mvk (M. paludicola) strain was obtained. A map of the modified genome of Δcrt::pAH162-P_{tac}-mvk (M. paludicola) is shown in FIG. 15A.

Subsequently, a genetic trait of SC17(0) Δcrt::pAH162-P_{tac}-mvk (M. paludicola) was transferred to SC17(0) ΔampC::attBₚₕᵢ₈₀ ΔampH::attBₚₕᵢ₈₀ via a genome DNA electroporation method (Katashkina J I et al., BMC Mol Biol., 2009; 10: 34). The resulting strain utilizes a tetracycline resistant gene, tetRA as the marker. Vector part of the pAH162-Ptac-mvk (M. paludicola) integrative plasmid including tetRA marker genes was eliminated using the reported pMW-intxis-cat helper plasmid (Katashkina J I et al., BMC Mol Biol., 2009; 10: 34, which is incorporated herein by reference in its entirety). As a result, SC17(0) ΔampH::attB_{φ80} ΔampC::attB_{φ80} Δcrt::P_{tac}-mvk (M. paludicola) with deletion of the marker gene was obtained. A map of the modified genome of Δcrt::P_{tac}-mvk (M. paludicola) is shown in FIG. 15B.

### 5-4) Construction of Set of SWITCH Strains

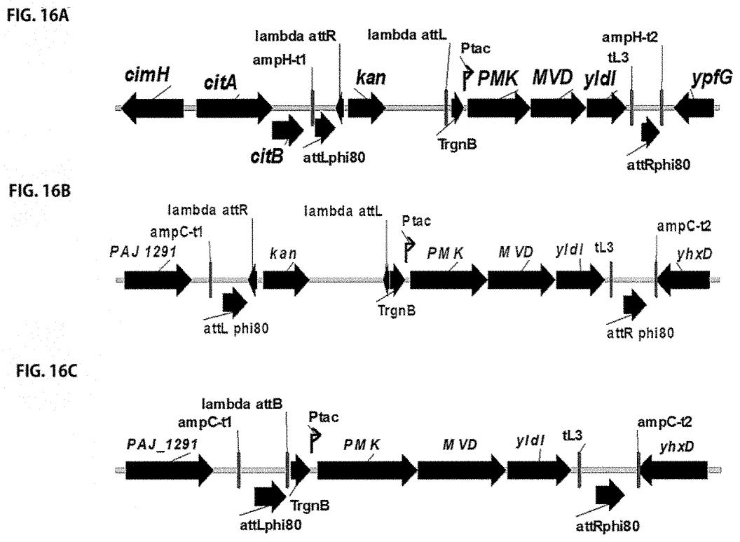

The pAH162-Km-Ptac-KDyI plasmid was integrated into a chromosome of SC17(0) ΔampH::attB_{φ80} ΔampC::attB_{φ80} Δcrt::P_{tac}-mvk (M. paludicola)/pAH123-cat strain according to the reported protocol (Andreeva I G et al., FEMS Microbiol Lett. 2011; 318(1): 55-60, which is incorporated herein by reference in its entirety). The cells were seeded on an LB agar containing 50 mg/L of kanamycin. A grown Km^{R} clone was examined by PCR using the primers 11 and 15 and the primers 11 and 17 (Table 2). Strains retaining the pAH162-Km-Ptac-KDyI plasmid integrated into ΔampH::attB_{φ80} or ΔampC::attB_{φ80}m were chosen. Maps of the modified chromosomes of ΔampH::pAH162-Km-Ptac-KDyI and ΔampC::pAH162-Km-Ptac-KDyI are shown in FIGS. 16A and 16B.

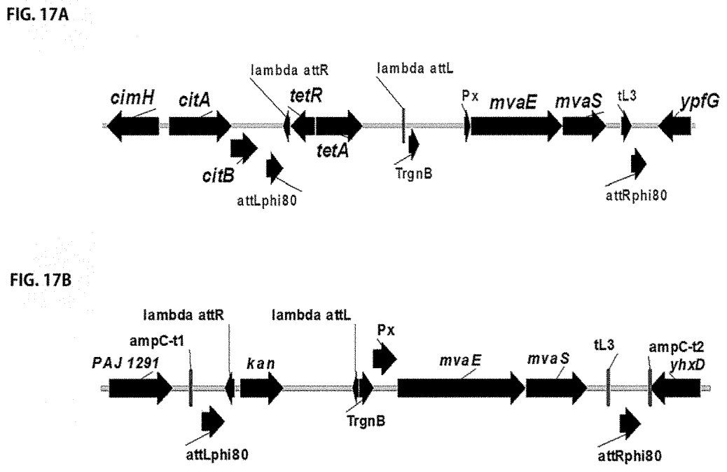

pAH162-Px-mvaES (here, Px is one of the following regulatory regions: araC-Para ara (E. coli), P_{lldD}, P_{phoC}, P_{pstS}) was inserted into the attBₚₕᵢ₈₀ site of SC17(0) ΔampC::pAH162-Km-P_{tac}-KDyI ΔampH::attBₚₕᵢ₈₀ Δcrt::P_{tac}-mvk (M. paludicola) and SC17(0) ΔampC::attBₚₕᵢ₈₀ ΔampH::pAH162-Km-P_{tac}-KDyI Δcrt::P_{tac}-mvk (M. paludicola) recipient strains using a pAH123-cat helper plasmid according to the reported protocol (Andreeva I G et al., FEMS Microbiol Lett., 2011; 318(1): 55-60, which is incorporated herein by reference in its entirety). As a result, two sets of strains designated as SWITCH-Px-1 and SWITCH-Px-2 were obtained. Maps of the modified chromosomes of ΔampH::pAH162-Px-mvaES and ΔampC::pAH162-Px-mvaES are shown in FIGS. 17A and 17B.

TABLE 2
Primer sequences utilized in Example 5

| No | Name | Sequence 5′->3′ |
|---|---|---|
| 1 | Linker-F | AGCTTTAGGGATAACAGGGTAATCTCGAGCTGCAGGCATGCA |
| | | (SEQ ID NO: 46) |
| 2 | Linker-R | AGCTTGCATGCCTGCAGCTCGAGATTACCCTGTTATCCCTAA |
| | | (SEQ ID NO: 47) |
| 3 | lldD5′CAS | TTTTTAAGCTTTAGGGATAACAGGGTAATCTCGAGATTTAAAGC |
| | | GGCTGCTTTAC (SEQ ID NO: 48) |
| 4 | lldD3′CAS | TTTTTAAGCTTGCATGCCTGCAGTATTTAATAGAATCAGGTAG |
| | | (SEQ ID NO: 49) |
| 5 | phoC5′CAS | TTTTTAAGCTTTAGGGATAACAGGGTAATCTCGAGTGGATAACC |
| | | TCATGTAAAC (SEQ ID NO: 50) |
| 6 | phoC3′CAS | TTTTTAAGCTTGCATGCCTGCAGTTGATGTCTGATTATCTCTGA |
| | | (SEQ ID NO: 51) |
| 7 | pstS5′CAS | TTTTTAAGCTTTAGGGATAACAGGGTAATCTCGAGAGCCTCTCA |
| | | CGCGTGAATC (SEQ ID NO: 52) |
| 8 | pstS3′CAS | TTTTTAAGCTTGCATGCCTGCAGAGGGGAGAAAAGTCAGGCTA |
| | | A (SEQ ID NO: 53) |
| 9 | n67 | TGCGAAGACGTCCTCGTGAAGAAGGTGTTGCTG (SEQ ID NO: 54) |
| 10 | n68 | TGCGAAGGGCCCCGTTGTGTCTCAAAATCTCTGATG |
| | | (SEQ ID NO: 55) |
| 11 | ampH- | ATGCGCACTCCTTACGTACTGGCTCTACTGGTTTCTTTGCGAAA |
| | attL-phi80 | GGTCATTTTTCCTGAATATGCTCACA (SEQ ID NO: 56) |
| 12 | ampH- | TTAAGGAATCGCCTGGACCATCATCGGCGAGCCGTTCTGACGTT |
| | attR-phi80 | TGTTGACAGCTGGTCCAATG (SEQ ID NO: 57) |
| 13 | DampC- | CTGATGAACTGTCACCTGAATGAGTGCTGATGAAAATATAGAA |
| | phL | AGGTCATTTTTCCTGAATATGCTCA (SEQ ID NO: 58) |
| 14 | DampC- | ATTCGCCAGCATAACGATGCCGCTGTTGAGCTGAGGAACACGT |
| | phR | TTGTTGACAGCTGGTCCAATG (SEQ ID NO: 59) |
| 15 | ampH-t1 | GCGAAGCCCTCTCCGTTG (SEQ ID NO: 60) |
| 16 | ampH-t2 | AGCCAGTCAGCCTCATCAGCG (SEQ ID NO: 61) |
| 17 | ampC-t1 | GATTCCCACTTCACCGAGCCG (SEQ ID NO: 62) |
| 18 | ampC-t2 | GGCAGGTATGGTGCTCTGACG (SEQ ID NO: 63) |
| 19 | crtE- | ATGACGGTCTGCGCAAAAAAACACGTTCATCTCACTCGCGCGT |
| | attRphi80 | TTGTTGACAGCTGGTCCAATG (SEQ ID NO: 64) |
| 20 | crtZ- | ATGTTGTGGATTTGGAATGCCCTGATCGTTTTCGTTACCGGAAA |
| | attLphi80 | GGTCATTTTTCCTGAATATGCTCA (SEQ ID NO: 65) |
| 21 | crtZ-test | CCGTGTGGTTCTGAAAGCCGA (SEQ ID NO: 66) |
| 22 | crtE-test | CGTTGCCGTAAATGTATCCGT (SEQ ID NO: 67) |
| 23 | phL-test | GGATGTAAACCATAACACTCTGCGAAC (SEQ ID NO: 68) |
| 24 | phR-test | GATTGGTGGTTGAATTGTCCGTAAC (SEQ ID NO: 69) |

### 5-5) Introduction of Isoprene Synthase Expression Plasmid

Competent cells of SWITCH strains were prepared according to a standard method, and pSTV28-Ptac-IspSM (WO 2013/179722, which is incorporated herein by reference in its entirety) that was an expression vector for isoprene synthase derived from mucuna was introduced thereto by the electroporation. The resulting isoprenoid compound-forming microorganisms were designated as SWITCH-Para/IspSM, SWITCH-Plld/IspSM, SWITCH-PpstS/IspSM, and SWITCH-PphoC/IspSM.

### Example 6: Cultivation of Phosphate Deficiency-Inducible Isoprenoid Compound-Forming Microorganisms SWITCH-PphoC/IspSM and SWITCH-PpstS/IspSM and Arabinose-Inducible Isoprenoid Compound-Forming Microorganism SWITCH-Para/IspSM

### 6-1) Cultivation of Isoprenoid Compound-Forming Microorganisms (SWITCH-Para/ispSM, SWITCH-PphoC/ispSM, SWITCH-PpstS/ispSM)

A fermentation jar having a 1 L volume was used for the cultivation of the isoprenoid compound-forming microorganisms (SWITCH-Para/ispSM, SWITCH-PphoC/ispSM, SWITCH-PpstS/ispSM). The glucose medium was prepared in the composition shown in Table 3. Each of the isoprenoid compound-forming microorganism was applied onto an LB plate containing chloramphenicol (60 mg/L), and cultured at 34° C. for 16 hours. After adding 0.3 L of the glucose medium into the fermentation jar having the 1 L volume, The microbial cells sufficiently grown on one plate were inoculated thereto, and the culture was started. The culture was carried out under a condition at pH 7.0 (controlled by ammonia gas) at 30° C., and air was supplied at 150 mL/minute. When an aerobic cultivation was carried out, the dissolved oxygen (DO) in the culture medium was measured using the galvanic mode DO sensor SDOU model (supplied from ABLE & Biott Co., Ltd), and controlled by stirring so that DO was a given concentration. During the cultivation, a solution of glucose adjusted to 500 g/L was continuously added so that a glucose concentration in the culture medium was 10 g/L or more. An OD value was measured at 600 nm using the spectrophotometer (HITACHI U-2900).

TABLE 3

| | Final concentration | |
|---|---|---|
| Group A | | |
| Glucose | 80 | g/L |
| MgSO₄•7aq | 2.0 | g/L |
| Group B | | |
| (NH₄)₂SO₄ | 2.0 | g/L |
| KH₂PO₄ | 2.0 | g/L |
| FeSO₄•7aq | 20 | mg/L |
| MnSO₄•5aq | 20 | mg/L |
| Yeast Extract | 4.0 | g/L |

Each 0.15 L of Group A and Group A was prepared, and then sterilized with heat at 115° C. for 10 minutes. After cooling, Group A and Group B were mixed, and chloramphenicol (60 mg/L) was added to use as the medium.

### 6-2) Method of Inducing Isoprene Production Phase

In an arabinose-inducible isoprenoid compound-forming microorganism, genes upstream of the mevalonate pathway are expressed by an arabinose inducible promoter, and thus an amount of isoprene produced in the presence of L-arabinose (Wako Pure Chemical Industries, Ltd.) is notably enhanced. To induce to an isoprene production phase, a broth in the fermentation jar was analyzed with time, and L-arabinose was added so that its final concentration was 20 mM at a time point when the OD value was 16.

In a phosphorus e deficiency-inducible isoprenoid compound-forming microorganism, genes upstream of the mevalonate pathway are expressed by a phosphorus deficiency-inducible promoter, and thus an amount of isoprene produced is notably enhanced when a concentration of phosphorus in the culture medium becomes a certain concentration or below.

### 6-3) Method of Measuring Concentration of Isoprene in Fermentation Gas and Method of Measuring Concentration of Total Phosphorus in Culture Medium

The isoprene concentration in the fermentation gas was a multi-gas analyzer (F10, supplied from GASERA). The concentration of total phosphorus in the culture medium was measured using a phosphate C-Test Wako (Wako Pure Chemical Industries Ltd.).

### 6-4) Amounts of Isoprene Formed in Jar Culture of Arabinose-Inducible Isoprenoid Compound-Forming Microorganism and Phosphorus Deficiency-Inducible Isoprenoid Compound-Forming Microorganism

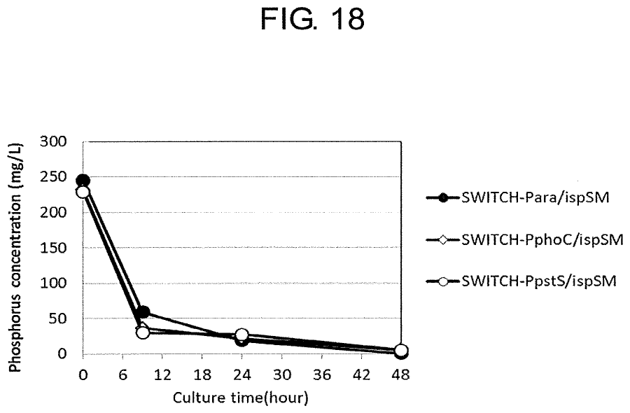

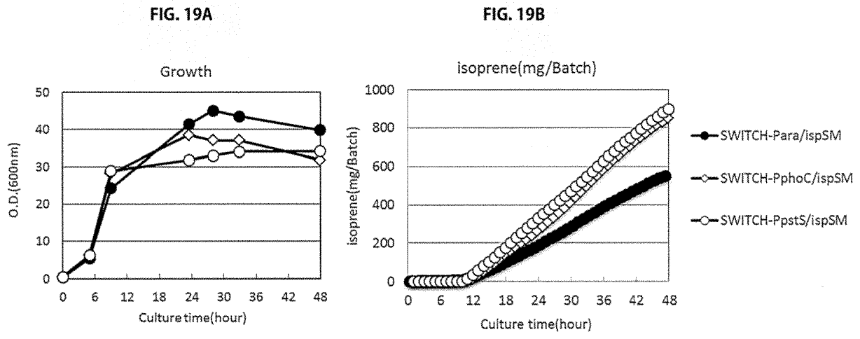

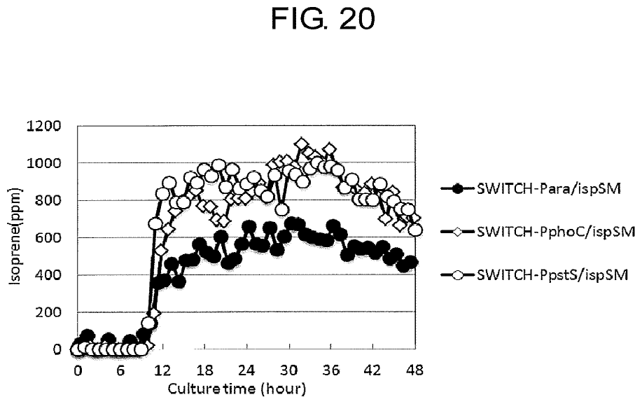

The arabinose-inducible isoprenoid compound-forming microorganism (SWITCH-Para-ispSM) and the phosphorus deficiency-inducible isoprenoid compound-forming microorganisms (SWITCH-PphoC/ispSM, SWITCH-PpstS/ispSM) were cultured under the above jar culture condition, and amounts of formed isoprene (mg/batch) and the isoprene concentration (ppm) in the fermentation gas were measured (FIGS. 19A, 19B and 20). During the cultivation, as shown in FIG. 18, the concentration of total phosphorus became 50 mg/L or less at 9 hours after starting the cultivation, and the production of isoprene was detected at the same timing in SWITCH-PphoC/ispSM and SWITCH-PpstS/ispSM. A period of time required from the start of the isoprene formation to a time at which a maximum rate of the isoprene formation was observed was shorter, and the formation rate increased more rapidly in SWITCH-PphoC/ispSM and SWITCH-PpstS/ispSM than in SWITCH-Para/ispSM (FIGS. 19A and 19B). The amounts of isoprene formed for 48 hours of the cultivation were 563 mg, 869 mg, and 898 mg in SWITCH-Para/ispSM, SWITCH-PphoC/ispSM and SWITCH-PpstS/ispSM, respectively (FIGS. 19A and 19B). This results indicated that the phosphorus deficiency-inducible isoprenoid compound-forming microorganism induced the isoprene formation under the condition where the concentration of phosphorus was 50 mg/L or less and had a more excellent ability to produce isoprene than the arabinose-inducible isoprenoid compound-forming microorganism.

### Example 7: Cultivation of Microaerphilically Inducible Isoprenoid Compound-Forming Microorganism (SWITCH-Plld/IspSM) and Cultivation of Arabinose-Inducible Isoprenoid Compound-Forming Microorganism (SWITCH-Para/IspSM)

### 7-1) Cultivation of Isoprenoid Compound-Forming Microorganisms (SWITCH-Para/ispSM, SWITCH-Plld/ispSM)

The isoprenoid compound-forming microorganisms (SWITCH-Para/ispSM, SWITCH-Plld/ispSM) were cultured in the same condition as in 6-1) above.

### 7-2) Method of Inducing Isoprene Production Phase

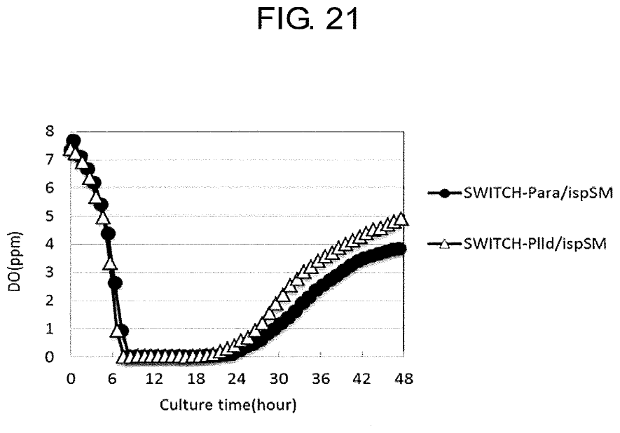

In the microaerobically inducible isoprenoid compound-forming microorganism (SWITCH-lld/ispSM), the isoprene-production phase was induced by supplying an air containing 20% (v/v) oxygen into the culture medium and regulating the stirring frequency during the cultivation to make the dissolved oxygen in the culture medium to be DO≈0 ppm. Changes with time of the dissolved oxygen concentration in the culture medium are shown in FIG. 21.

### 7-3) Method of Measuring Isoprene Concentration in Fermentation Gas and Method of Measuring Dissolved Oxygen in Culture Medium

The isoprene concentration in the fermentation gas was the multi-gas analyzer (F10, supplied from GASERA). The dissolved oxygen concentration in the culture medium was measured using the galvanic mode DO sensor SDOU model (supplied from ABLE & Biott Co., Ltd).

### 7-4) Amounts of Isoprene Formed in Jar Culture of Arabinose-Inducible Isoprenoid Compound-Forming Microorganism and Microaerobically Inducible Isoprenoid Compound-Forming Microorganism.

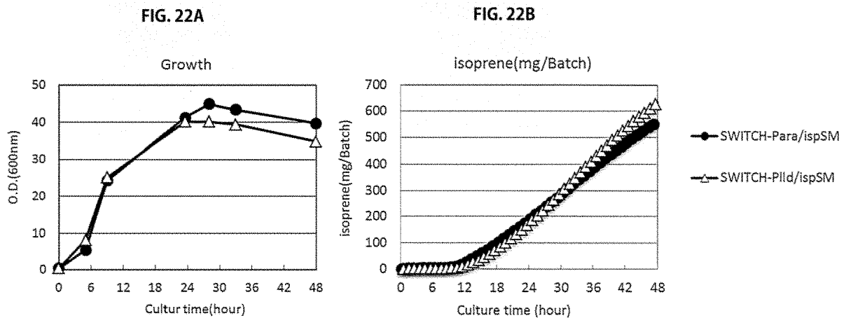

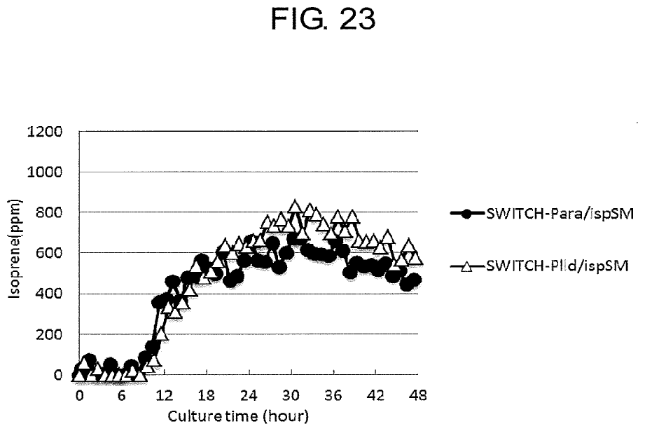

The arabinose-inducible isoprenoid compound-forming microorganism (SWITCH-Para/ispSM) and the microaerobically inducible isoprenoid compound-forming microorganism (SWITCH-lld-ispSM) were cultured under the above jar culture condition, and the amounts of formed isoprene (mg/batch) and the isoprene concentration (ppm) in the fermentation gas were measured (FIGS. 22A, 22B and 23). As shown in FIG. 21, the dissolved oxygen concentration in the culture medium reached DO≈0 ppm at 8 hours after starting the cultivation, and shortly after, the production of isoprene was detected. The amounts of isoprene formed for 48 hours of the cultivation were 563 mg and 642 mg in SWITCH-Para/ispSM and SWITCH-Plld/ispSM, respectively (FIGS. 22A and 22B). This result indicated that the microaerobically inducible isoprenoid compound-forming microorganism induced the formation of isoprene under the condition of DO ppm or less and had the ability to produce isoprene, which was equivalent to that of the arabinose-inducible isoprenoid compound-forming microorganism.

### Reference Example 1) Construction of E. coli MG1655 Ptac-KDyI Strain

E. coli MG1655 Ptac-KDyI strain was made by deleting an ERG12 gene in MG1655 Ptac-KKDyI strain (see Example 7-5 in WO 2013/179722, which is incorporated herein by reference in its entirety). A specific procedure is as follows.

A plasmid pKD46 having a temperature sensitive replication capacity was introduced into MG1655 Ptac-KKDyI strain by the electroporation method. The plasmid pKD46 (Proc. Natl. Acad. Sci. USA, 2000, vol. 97, No. 12, p 6640-6645, which is incorporated herein by reference in its entirety) contains a DNA fragment of total 2154 bases of λ phage (GenBank/EMBL Accession Number: J02459, 31088^{th} to 33241^{st}) containing genes (λ, β, exo genes) of a X-Red system controlled by an arabinose-inducible ParaB promoter. Competent cells of MG1655 Ptac-KKDyI strain were prepared, and then pKD46 was introduced thereto by the electroporation method. The cells were evenly applied onto an LB plate containing ampicillin (100 mg/L), and cultured at 37° C. for 18 hours. Subsequently, transformants exhibiting ampicillin resistance were obtained from the resulting plate. A strain in which pKD46 had been introduced into E. coli MG1655 Ptac-KDDyI strain was designated as MG1655 Ptac-KDDyI/pKD46. PCR was carried out with attL-tetR-attR-Ptac gene fragment (SEQ ID NO:38 in WO2013/179722) as the template using synthesized oligonucleotides consisting of SEQ ID NO:39 and SEQ ID NO:40 in WO2013/179722 and using Prime Star polymerase (supplied from Takara Bio Inc.). A reaction solution was prepared according to the composition attached to the kit, and DNA was amplified through 30 cycles of reactions at 98° C. for 10 seconds, 55° C. for 5 seconds and 72° C. for one minute per kb. As a result, an MVK gene deficient fragment containing attL-tetR-attR-Ptac was obtained. Competent cells of MG1655 Ptac-KDDyI/pKD46 were prepared, and then the purified MVK gene deficient fragment containing attL-tetR-attR-Ptac was introduced thereto by the electroporation method. After the electroporation, a colony that had acquired tetracycline resistance was obtained. PCR reaction was carried out using synthesized oligonucleotides consisting of SEQ ID NO:41 and SEQ ID NO:42 in WO 2013/179722, which is incorporated herein by reference in its entirety, as the primers to confirm that the ERG12 gene on the chromosome was deficient. The obtained mutant was designated as E. coli MG1655 Ptac-KDyI.

### Reference Example 2) Construction of Arabinose-Inducible mvaES Expression Vector (pMW-Para-mvaES-Ttrp)

An arabinose-inducible expression vector for mevalonate pathway upstream genes was constructed by the following procedure. A PCR fragment containing Para consisting of araC and araBAD promoter sequences derived from E. coli was obtained by PCR with the plasmid pKD46 as the template using synthesized oligonucleotides represented by SEQ ID NO:49 and SEQ ID NO:50 in WO 2013/179722, which is incorporated herein by reference in its entirety, as the primers. A PCR fragment containing the EFmvaE gene was obtained by PCR with the plasmid pUC57-EFmvaE as the template using the synthesized oligonucleotides represented by SEQ ID NO:51 and SEQ ID NO:52 in WO 2013/179722, which is incorporated herein by reference in its entirety as the primers. A PCR fragment containing the EFmvaS gene was obtained by PCR with the plasmid pUC57-EFmvaS as the template using the synthesized oligonucleotides represented by SEQ ID NO:53 and SEQ ID NO:54 in WO 2013/179722, which is incorporated herein by reference in its entirety, as the primers. A PCR fragment containing a Ttrp sequence was obtained by PCR with the plasmid pSTV-Ptac-Ttrp as the template (source of the plasmid) using the synthesized oligonucleotides represented by SEQ ID NO:55 and SEQ ID NO:56 in WO 2013/179722, which is incorporated herein by reference in its entirety, as the primers. Prime Star polymerase (TAKARA BIO Inc.) was used for PCR for obtaining these four PCR fragments. Reaction solutions were prepared according to the composition attached to the kit, and DNA was amplified through 30 cycles of the reactions at 98° C. for 10 seconds, 55° C. for 5 seconds and 72° C. for one minute per kb. PCR with the purified PCR product containing Para and the PCR product containing the EFmvaE gene as the template was carried out using the synthesized oligonucleotides represented by SEQ ID NO:49 and SEQ ID NO:52 in WO 2013/179722, which is incorporated herein by reference in its entirety, as the primers. PCR with the purified PCR product containing the EFmvaS gene and the PCR product containing Ttrp as the template was also carried out using the synthesized oligonucleotides represented by SEQ ID NO:53 and SEQ ID NO:56 in WO2013/179722, which is incorporated herein by reference in its entirety, as the primers. As a result, a PCR product containing Para and the EFmvaE gene and a PCR product containing the EFmvaS gene and Ttrp were obtained. A plasmid pMW219 (supplied from Nippon Gene Co., Ltd.) was digested with SmaI according to a standard method. Then, pMW219 after being digested with SmaI was ligated to the PCR product containing Para and the EFmvaE gene and the PCR product containing the EFmvaS gene and Ttrp using In-Fusion HD Cloning Kit (supplied from Clontech). The obtained plasmid was designated as pMW-Para-mvaES-Ttrp.

### Phosphate Starvation Induction

### Background

In this study, the transfer from a cell growth phase to a substance-production phase is realized by a metabolic switch to respond to phosphate starvation. Generally, an optimal metabolic condition is different between the growth phase and the substance-production phase. Conventionally, methods of optimizing the metabolic condition in each phase have been known, but even if a culture condition optimal for the growth phase is switched to a culture condition optimal for the substance production phase, this switch often does not work well due to reasons such as reduced cellular activity and the like. It has been known that the decrease of a phosphate concentration in a cell reduces an acquired amount of ATP (Schuhmacher, T., Loffler, M., Hurler, T., Takors, R., 2014. Phosphate limited fed-batch processes: Impact on carbon usage and energy metabolism in Escherichia coli, J. Biotechnol., doi: 10.1016/j.jbiotec.2014.04.025, which is incorporated herein by reference in its entirety). Thus, the decrease of the phosphate concentration is predicted to reduce a production rate of a metabolite in the production of the metabolite that requires a high ATP amount.

Multiple stages of phosphate reactions using ATP as a substrate are present in the mevalonate pathway that is a formation pathway of isoprene (Michelle C Y Chang & Jay D Keasling, Production of isoprenoid pharmaceuticals by engineered microbes, Nature Chemical Biology 2, 674-681 (2006) which is incorporated herein by reference in its entirety). Thus, isoprene fermentation is thought to be the production of the metabolite that requires the high APT amount. Therefore, it was easily feared that the cultivation of an isoprene-producing strain having the mevalonate pathway at low phosphate concentration caused a decreased amount of produced isoprene.

It has been known in literatures that the response to the phosphate starvation rapidly acts upon expression control of a gene (Baek J H et al., J Microbiol Biotechnol. 2007 February; 17 (2): 244-52; WO 2003/054140 A2, which are incorporated herein by reference in their entireties). However, it cannot be easily expected that the metabolic condition is rapidly switched by the response to the phosphate starvation and a quick response is observed at level of substance production under a condition where inhibition and the like at enzymatic level are known in combination of the response to the phosphate starvation with control of constitutively expressed genes.

### Example of Effect Observed by Study

The higher concentration of isoprene was identified in the phosphate starvation-inducible isoprene-producing strain constructed by us than the arabinose-inducible isoprene-producing strain that was a control.

Also as an unexpected effect, rapid responsiveness (time required to reach a maximum isoprene concentration) was observed (Example 6, FIGS. 19A and 19B). IPTG has been mainly used as the inducer in previous cases of the isoprene fermentation (U.S. Pat. No. 8,288,148 B2; U.S. Pat. No. 8,361,762 B2; U.S. Pat. No. 8,470,581 B2; U.S. Pat. No. 8,569,026 B2; U.S. Pat. No. 8,507,235 B2; U.S. Pat. No. 8,455,236 B2; US 2010-0184178 A1, which are incorporated herein by reference in their entireties). In these cases, it takes about 8 to 22 hours for an ability per microbial cell to produce isoprene to reach the maximum or for an accumulated isoprene to reach the maximum after adding the inducer. On the contrary, when the technique for the phosphate starvation shown in Example 6, the isoprene gas concentration in the reactor reached the maximum within 3 to 6 hours.

TABLE 4
Comparison of responsiveness by difference of technique for induction

| | | Induction method | | | |
|---|---|---|---|---|---|
| | | Arabinose | Microaerophilic | Phosphorus deficiency | |
| | | SWITCH- | SWITCH- | SWITCH- | SWITCH- |
| | | Para/IspSM | Plld/IspSM | PphoC/IspSM | PpstS/IspSM |
| | | Example 7 | Example 7 | Example 6 | Example 6 |
| Induction time | hour | 21 | 21 | 6 | 3 |
| Induction index | ppm/vvm/h | 64.2 | 79 | 283 | 595 |

Induction time: Time from start of isoprene formation (defined as concentration of 50 ppm) to maximum.
Induction index: Value obtained by dividing maximum isoprene concentration (ppm/vvm) by induction time
vvm: Volume per volume per minute (in the case of ventilation stirring, ventilation amount of gas per unit volume)

### Microaerophilic Induction

### Background

In this study, the transfer from the cell growth phase to the substance-production phase is realized by change of metabolism caused by deficiency of the dissolved oxygen. Generally, it has been known that the metabolic condition in the cell is largely different between the culture condition where oxygen is available for a microorganism and the culture condition where oxygen is not available for the microorganism (Martinez I., Bennett G. N., San K. Y. 2010. Metabolic impact of the level of aeration during cell growth on anaerobic succinate production by an engineered Escherichia coli strain. Metab. Eng. 12:499-509, which is incorporated herein by reference in its entirety). Conventionally, methods of optimizing the metabolic condition under an aerobic condition and an anaerobic condition have been known. In order to perform the fermentation under an essentially anaerobic condition such as succinate and alcohol fermentation, it is often studied that applying this, the culture condition optimal for the growth phase is switched to the culture condition optimal for the substance production phase by changing the oxygen concentration in the cultivation (Blombach B, Riester T, Wieschalka S, Ziert C, Youn J W, Wendisch V F, Eikmanns B J. Corynebacterium glutamicum tailored for efficient isobutanol production. Appl Environ Microbiol. 2011 May; 77(10): 3300-10, which is in incorporated herein by reference in its entirety). On the contrary, in the fermentation that requires that excess reducing capacity such as isoprene and glutamic acid is re-oxidized by oxygen respiration, the condition where the dissolved oxygen is deficient is not regarded as the condition that leads to the metabolic condition suitable for the substance production phase. Thus, this method is not the method of transferring from the cell growth phase to the substance production phase, which is actively employed by sector peer companies.

The method in more detail is as follows. Under the aerobic condition, typically oxygen works as a terminal electron acceptor, thereby NADH is reoxidized (respiration). Under a low oxygen concentration environment such as a microaerophilic condition, an amount of supplied oxygen is a limiting factor, and an NADH concentration in a cell is increased. In E. coli, synthesis pathways for lactic acid and ethanol are present as reoxidation reaction of this excess NADH, and NADH is reoxidized in processes of producing these substances. Likewise in P. ananatis, 2,3-butandiol, lactic acid, ethanol, and the like are synthesized under the low oxygen concentration environment to keep a balance between oxidation and reduction. That is, under the low oxygen concentration environment, metabolic flux to these substances is increased, and thus it is presumed that the amount of produced isoprene is decreased. When isoprene is produced via the mevalonate pathway, it is evident from calculation of a theoretic yield that excessive NADH is produced (Yadav G V et al., The future of metabolic engineering and synthetic biology: Towards a systematic practice, Metabolic Engineering, 14, 233-241, 2012, which is incorporated herein by reference in its entirety). Typically, oxygen is needed for this reoxidation of NADH, and thus a person skilled in the art does not allow himself/herself to select the culture under the low oxygen concentration environment. In fact, in the study on isoprene production by Saccharomyces cerevisiae, it has been known that the growth of microbial cells and the ability to produce isoprene are enhanced under the aerobic condition where the dissolved oxygen is sufficiently supplied than in the microaerophilic condition where the dissolved oxygen is deficient, as a result of the comparative study of the culture conditions (Lv X et al., Journal of Biotechnology, 186, 128-136, 2014, which is incorporated herein by reference in its entirety).

### Example of Effect Observed by Study

When E. aerogenes was used as a parent strain of the isoprene-producing strain, it was demonstrated that GI08-Pbud/IspSK could successfully switch the growth phase to the isoprene-production phase when the dissolved oxygen (DO) concentration was almost zero (Example 4, FIGS. 4A and 4B).

When P. ananatis was used as a parent strain of the isoprene-producing strain, the growth phase was switched to the isoprene production phase by exposing to the condition where the dissolved oxygen (DO) concentration was almost zero, and this phase was transferred to the metabolic condition suitable for the isoprene production by increasing the dissolved oxygen concentration again in the isoprene production phase (Example 7, FIGS. 22A, 22B and 23). As shown in FIG. 23, the higher isoprene concentration than that in the arabinose-inducible isoprene-producing strain that was the control was confirmed.

### Example 8: Production of Polyisoprene

Isoprene is collected with a trap cooled with liquid nitrogen by passing the fermentation exhaust. Collected of isoprene is mixed with 35 g of hexane (Sigma-Aldrich, catalog No.) and 10 g of silica gel (Sigma-Aldrich, catalog No. 236772) and 10 g of alumina (Sigma-Aldrich, catalog No. 267740) under a nitrogen atmosphere in 100 mL glass vessel that is sufficiently dried. Resulting mixture is left at room temperature for 5 hours. Then supernatant liquid is skimmed and is added into 50 ml glass vessel that is sufficiently dried.

Meanwhile, in a glove box under a nitrogen atmosphere, 40.0 μmol of Tris[bis(trimethylsilyl)amido]gadrinium, 150.0 μmol of tributylaluminium, 40.0 μmol of Bis[2-(diphenylphosphino)phenyl]amine, 40.0 μmol of triphenylcarbonium tetrakis(pentafluorophenyl)borate (Ph3CBC6F5)4) are provided in a glass container, which was dissolved into 5 mL of toluene (Sigma-Aldrich, catalog No. 245511), to thereby obtain a catalyst solution. After that, the catalyst solution is taken out from the glove box and added to the monomer solution, which is then subjected to polymerization at 50° C. for 120 minutes.

After the polymerization, 1 mL of an isopropanol solution containing, by 5 mass %, 2,2′-methylene-bis(4-ethyl-6-t-butylphenol) (NS-5), is added to stop the reaction. Then, a large amount of methanol is further added to isolate the copolymer, and the copolymer is vacuum dried at 70° C. to obtain a polymer.

### Example 9: Production of Rubber Compound

The rubber compositions formulated as shown in Table 5 are prepared, which are vulcanized at 145° C. for 35 minutes.

TABLE 5

| | Parts by Mass |
|---|---|
| Polyisoprene | 100 |
| Stearic Acid | 2 |
| Carbon Black (HAF class) | 50 |
| Anti Oxidant (*1) | 1 |
| Zinc Oxide | 3 |
| Cure Accelerator (*2) | 0.5 |
| Sulfur | 1.5 |

(*1) N-(1,3-dimethylbutyl)-N′-p-phenylenediamine
(*2) N-cyclohexyl-2-benzothiazolesulfenamide

### Example 10: Construction of SC17(0)ΔGcd and SWITCH-PphoC ΔGcd Strains, and Introduction of Isoprene Synthase

The gcd gene in P. ananatis codes for glucose dehydrogenase, and it has been known that P. ananatis accumulates gluconate during aerobic growth (Andreeva I G et al., FEMS Microbiol Lett. 2011 May; 318(1):55-60, which is incorporated herein by reference in its entirety).

The SC17(0)Δgcd strain in which gcd gene is disrupted is constructed using λRed-dependent integration of DNA fragments obtained in PCRs with the primers gcd-attL and gcd-attR (Table 6) and pMW118-attL-kan-attR plasmid (Minaeva N I et al., BMC Biotechnol. 2008; 8:63, which is incorporated herein by reference in its entirety) as a template. To verify the integrant, the primers gcd-t1 and gcd-t2 (Table 6) are used.

Genomic DNA of the SC17(0)Δgcd strain is isolated using the Wizard Genomic DNA Purification Kit (Promega) and electro-transformed into the marker-less derivative of the SWITCH-PphoC strain according to the previously described method (Katashkina J I et al., BMC Mol Biol. 2009; 10:34, which is incorporated herein by reference in its entirety). As a result, the SWITCH-PphoC Δgcd (KmR) strain is obtained. The primers gcd-t1 and gcd-t2 (Table 6) are used for PCR analysis of the obtained integrant. The kanamycin resistant marker gene is obtained according to the standard λInt/Xis-mediated procedure (Katashkina J I et al., BMC Mol Biol. 2009; 10:34, which is incorporated herein by reference in its entirety). The obtained strain is designated as SWITCH-PphoC Δgcd strain.

Competent cells of SWITCH-PphoC Δgcd strain was prepared according to a standard method, and pSTV28-Ptac-IspSM (WO2013/179722) that was an expression vector for isoprene synthase derived from mucuna was introduced thereto by the electroporation. The resulting isoprenoid compound-forming microorganisms were designated as SWITCH-PphoC Δgcd/IspSM.

TABLE 6
Primer List

| Primer | Nucleotide sequence (seq ID no:) |
|---|---|
| gcd-attL | GGTCAACATTATGGGGAAAAACTCCTCATCCTTTAGCGTGtga |
| | agcctgattttttatactaagttgg (seq ID no: 71) |
| gcd-attR | TTACTTCTGGTCGGGCAGCGCATAGGCAATCACGTAATCGcgc |
| | tcaagttagtataaaaaagctgaac (seq ID no: 72) |
| gcd-t1 | TGACAACAATCTATCTGATT (seq ID no: 73) |
| gcd-t2 | tgcgcctggttaagctggcg (seq ID no: 74) |

### Example 11: Evaluation of Cultivation of SWITCH-PphoC ΔGcd/IspSM

### 11-1) Condition for Jar Cultivation of Isoprene-Producing Microorganism

A one liter volume fermenter was used for cultivation of isoprene-producing microorganisms (SWITCH-PphoC/IspSM and SWITCH-PphoCΔgcd/IspSM). Glucose medium was conditioned in a composition shown in Table 7. Each of these isoprene-producing microorganisms was applied onto an LB plate containing chloramphenicol (60 mg/L), and cultured at 34° C. for 16 hours. 0.3 L of the glucose medium was added to the one liter volume fermenter, and microbial cells that had sufficiently grown on the one LB plate were inoculated thereto to start the cultivation. As a culture condition, pH was 7.0 (controlled with ammonia gas), temperature was 30° C., and ventilation at 150 mL/minute was carried out. When aerobic cultivation was carried out, a concentration of oxygen in culture medium (dissolved oxygen (DO)) was measured using a galvanic type DO sensor SDOU model (ABLE Cooperation), and was controlled with stirring so that a DO value was 5%. During the cultivation, a glucose solution adjusted at 50 g/L was continuously added so that a concentration of glucose in the culture medium was 10 g/L or higher. The OD value was measured at 600 nm using a spectrophotometer (HITACHI U-2900). In the cultivation for 48 hours, SWITCH-PphoC/IspSM and SWITCH-PphoCΔgcd/IspSM consumed 63.9 g and 77.8 g of glucose, respectively.

TABLE 7
Composition of glucose medium

| | (Final concentration) | |
|---|---|---|
| Group A | | |
| Glucose | 80 | g/L |
| MgSO₄•7aq | 2.0 | g/L |
| Group B | | |
| (NH₄)₂SO₄ | 2.0 | g/L |
| KH₂PO₄ | 2.0 | g/L |
| FeSO₄•7aq | 20 | mg/L |
| MnSO₄•5aq | 20 | mg/L |
| Yeast Extract | 4.0 | g/L |

Each 0.15 L was prepared for Group A and Group B and sterilized with heating at 115° C. for 10 minutes. After cooling, Group A and Group B were mixed, and chloramphenicol (60 mg/L) was added thereto to use as the medium.

### 11-2) Method of Inducing Isoprene Production Phase

A phosphorus-deficient isoprene-producing microorganism expresses genes upstream of a mevalonate pathway with a phosphorus deficiency-inducible promoter. Thus, when a concentration of phosphorus in the medium is below a certain concentration, an amount of produced isoprene is remarkably increased.

### 11-3) Method of Measuring Isoprene Concentration in Fermentation Gas

A concentration of isoprene in fermentation gas was measured using a multi-gas analyzer (supplied from GASERA, F10).

### 11-4) Method of Measuring Gluconic Acid Concentration in Medium

Culture supernatant was diluted with pure water to 10 times, and filtrated through a 0.45 μm filter followed by being analyzed according to the following method using high performance liquid chromatography ELITE LaChrom (Hitachi High Technologies).

### Separation Conditions

Columns: Shim-pack SCR-102H (8 mm I.D.×300 mm L), tandemly connected two columns
Guard column: SCR-102H (6 mm I.D.×50 mm L)
Mobile phase: 5 mM p-toluenesulfonic acid
Flow: 0.8 mL/minute

### Temperature: 50° C.

Injection volume: 10 μL

### Detection Conditions

Buffer: 20 mM Bis-Tris aqueous solution containing 5 mM p-toluenesulfonic acid and 100 μM EDTA
Flow: 0.8 mL/minute
Detector: CDD-10 AD polarity+response SLOW, temperature: 53° C. (column
temperature: +3° C.); scale 1×2⁴ μS/cm

### 11-5) Amount of Produced Isoprene in Jar Cultivation of Isoprene-Producing Microorganisms (SWITCH-PphoC/IspSM and SWITCH-PphoCΔGcd/IspSM)

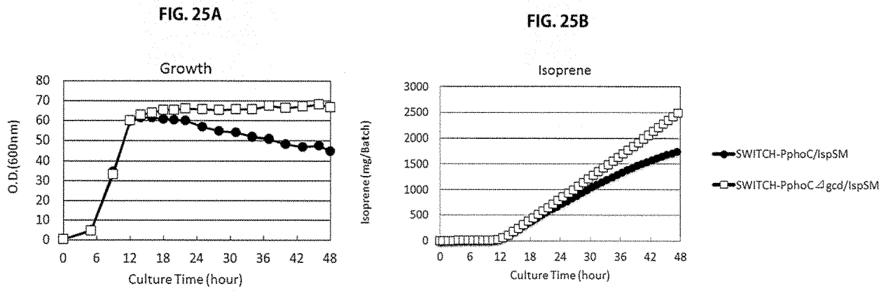

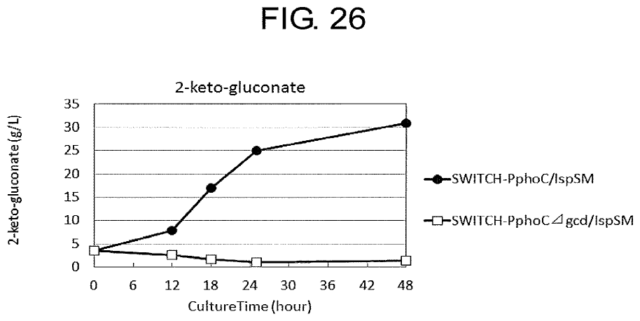

The isoprene-producing microorganisms (SWITCH-PphoC/IspSM and SWITCH-PphoCΔgcd/IspSM) were cultured according to the jar cultivation condition as described above, and amounts of produced isoprene were measured. SWITCH-PphoC/IspSM exhibited a decreased O.D. value when production of isoprene was started (FIG. 25A), and accumulated 30.9 g/L of 2-ketogluconic acid in the cultivation for 48 hours (FIG. 26). SWITCH-PphoCΔgcd/IspSM exhibited a constant O.D. value even after starting the production of isoprene (FIG. 25(A)), and an accumulated amount of 2-ketogluconic acid in the cultivation for 48 hours was 1.4 g/L, which was an extremely low amount (FIG. 26). The amounts of isoprene produced in the cultivation for 48 hours were 1771 mg and 2553 mg in SWITCH-PphoC/IspSM and SWITCH-PphoCΔgcd/IspSM, respectively (FIG. 25B). From this result, it was shown that the production of 2-ketogluconic acid was suppressed while the amount of produced isoprene was increased in SWITCH-PphoCΔgcd/IspSM.

### Example 12: Construction of Expression Plasmid for Linalool Synthase

12-1) Acquisition of Linalool Synthase Gene Derived from Actinidia arguta (Hardy Kiwifruit)

A nucleotide sequence (GenBank accession number: GQ338153) and an amino acid sequence (GenPept accession number: ADD81294) of a linalool synthase (AaLINS) gene derived from Actinidia arguta have been already known. The amino acid sequence of a linalool synthase protein and the nucleotide sequence of its gene derived from Actinidia arguta are shown in SEQ ID NO:75 and SEQ ID NO:76, respectively. In order to efficiently express the AaLINS gene, codons were optimized to resolve a secondary structure, an AaLINS gene in which a chloroplast localization signal had been cleaved was designed, and this was designated as opt_AaLINS. A nucleotide sequence of opt_AaLINS is shown in SEQ ID NO:77. DNA in which a tac promoter region (deBoer, et al., (1983) Proc. Natl. Acad. Sci. USA., 80, 21-25, which is incorporated herein by reference) had been added to the opt_AaLINS gene was chemically synthesized, cloned into pUC57 (supplied from GenScript) and the resulting plasmid was designated as pUC57-Ptac-opt_AaLINS.

12-2) Acquisition of Linalool Synthase Gene Derived from Coriandrum sativum (Coriander)

A nucleotide sequence (GenBank accession number: KF700700) and an amino acid sequence (GenPept accession number: AHC54051) of a linalool synthase (CsLINS) gene derived from Coriandrum sativum have been already known. The amino acid sequence of a linalool synthase protein and the nucleotide sequence of its gene derived from Coriandrum sativum are shown in SEQ ID NO:78 and SEQ ID NO:79, respectively. In order to efficiently express the CsLINS gene, codons were optimized to resolve a secondary structure, a CsLINS gene in which the chloroplast localization signal had been cleaved was designed, and this was designated as opt_CsLINS. A nucleotide sequence of opt_CsLINS is shown in SEQ ID NO:80. DNA in which the tac promoter region (deBoer, et al., (1983) Proc. Natl. Acad. Sci. USA., 80, 21-25, which is incorporated herein by reference in its entirety) had been added to the opt_CsLINS gene was chemically synthesized, cloned into pUC57 (supplied from GenScript), and the resulting plasmid was designated as pUC57-Ptac-opt_CsLINS.

12-3) Acquisition of Mutated Farnesyl Diphosphate Synthase Gene Derived from Escherichia coli

Farnesyl diphosphate synthase derived from Escherichia coli is encoded by an ispA gene (SEQ ID NO:81) (Fujisaki S., et al. (1990) J. Biochem. (Tokyo) 108:995-1000, which is incorporated herein by reference in its entirety). A mutation which increases a concentration of geranyl diphosphate in microbial cells has been demonstrated in farnesyl diphosphate synthase derived from Bacillus stearothermophilus (Narita K., et al. (1999) J Biochem 126(3):566-571, which is incorporated herein by reference in its entirety). Based on this finding, the similar mutant has been also produced in farnesyl diphosphate synthase derived from Escherichia coli (Reiling K K et al. (2004) Biotechnol Bioeng. 87(2) 200-212, which is incorporated herein by reference in its entirety). In order to efficiently express an ispA mutant (S80F) gene having a high activity for producing geranyl diphosphate, a sequence in which the codons were optimized to resolve the secondary structure was designed and designated as ispA*. A nucleotide sequence of ispA* is shown in SEQ ID NO:82. The ispA* gene was chemically synthesized, subsequently cloned into pUC57 (supplied from GenScript) and the resulting plasmid was designated as pUC57-ispA*.

12-4) Construction of Co-Expression Plasmid for Opt_AaLINS and ispA* Genes

PCR with pUC57-Ptac-opt_AaLINS as a template was carried out using primers shown in SEQ ID NO:83 and SEQ ID NO:85 to obtain a Ptac-opt_AaLINS fragment. Further, PCR with pUC57-ispA* as a template was carried out using primers shown in SEQ ID NO:86 and SEQ ID NO:87 to obtain an ispA* fragment. The purified Ptac-opt_AaLINS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_AaLINS-ispA*.

12-5) Construction of Co-Expression Plasmid for Opt_CsLINS and ispA* Genes

PCR with pUC57-Ptac-opt_CsLINS as a template was carried out using primers shown in SEQ ID NO:83 and SEQ ID NO:88 to obtain a Ptac-opt_CsLINS fragment. Further, PCR with pUC57-ispA* as a template was carried out using primers shown in SEQ ID NO:89 and SEQ ID NO:87 to obtain an ispA* fragment. The purified Ptac-opt_CsLINS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_CsLINS-ispA*.

### 12-6) Preparation of Linalool Production Strains

Competent cells of SWITCH-PphoCΔgcd were prepared, and pACYC177, pACYC177-Ptac-opt_AaLINS-ispA* or pACYC177-Ptac-opt_CsLINS-ispA* was introduced thereto by electroporation. Resulting strains were designated as SWITCH-PphoCΔgcd/pACYC177, SWITCH-PphoCΔgcd/AaLINS-ispA* and SWITCH-PphoCΔgcd/CsLINS-ispA*.

### Example 13: Evaluation of Ability to Produce Linalool by Linalool Synthase-Expressing Strains Derived from SWITCH-PphoCΔGcd Strain

Microbial cells of SWITCH-PphoCΔgcd/AaLINS-ispA*, SWITCH-PphoCΔgcd/CsLINS-ispA* and SWITCH-PphoCΔgcd/pACYC177 strains obtained in Example 12 in glycerol stock were thawed. Subsequently 50 μL of a microbial cell suspension from each strain was evenly applied onto an LB plate containing 50 mg/L of kanamycin, and cultured at 34° C. for 16 hours. The resulting microbial cells on the plate were picked up in an amount corresponding to about ¼ of a loop part of a 10 μL inoculating loop (supplied from Thermo Fisher Scientific Inc.). The picked up microbial cells were inoculated to 5 mL of fermentation medium described below containing 50 mg/L of kanamycin in a test tube supplied from AGC Techno Glass Co., Ltd. (diameter×length×thickness=25×200×1.2 mm), and cultured at 30° C. on a reciprocal shaking culture apparatus at 120 rpm for 24 hours.

TABLE 8
Fermentation medium for SWITCH-PphoCΔgcd,
host strain for production of linalool

| Group A | | |
|---|---|---|
| D-Glucose | 40 | g/L |
| MgSO₄•7H₂O | 1 | g/L |

Not adjusted pH, AC 115° C., 10 minutes

| | | |
|---|---|---|
| Group B | | |
| (NH₄)₂SO₄ | 20 | g/L |
| KH₂PO₄ | 0.5 | g/L |
| Yeast extract | 2 | g/L |
| FeSO₄•7H₂O | 0.01 | g/L |
| MnSO₄•5H₂O | 0.01 | g/L |

After adjusting pH to 7.0 with KOH, AC 115° C.,
10 minutes

| | | |
|---|---|---|
| Group C | | |
| CaCO₃ | 20 | g/L |

Dry-heat sterilization 180° C., 2 hours

After the sterilization, the above Groups A, B and C were mixed. Then, 1 mL of isopropyl myristate (Tokyo Chemical Industry Co., Ltd.) was added to 5 mL of the fermentation medium dispensed in the test tube.

Twenty-four hours after starting the cultivation, the concentrations of isopropyl myristate and linalool in the culture supernatant were measured under the following condition using gas chromatography GC-2025AF (supplied from Shimadzu Corporation). DB-5 (supplied from Agilent, length 30 m, internal diameter 0.25 mm, thickness 0.25 μm) was used as a column, and a linalool standard solution was prepared using a reagent Linalool (supplied from Wako Pure Chemical Industries Ltd.).

| Temperature in vaporization room | 360.0° | C. |
|---|---|---|
| Injection amount | 1.0 | μL |
| Injection mode | Split 1:10 | |
| Carrier gas | He | |
| Control mode | Line velocity | |
| Pressure | 125.5 | kPa |
| Total flow | 20.5 | mL/minute |
| Column flow | 1.59 | mL/minute |
| Line velocity | 36.3 | cm/sec |
| Purge flow | 3.0 | mL/minute |

Column open temperature program Total time 21.5 minutes

| Rate (° C./minute) | Temperature (° C.) | Hold time (min) |
|---|---|---|
| | 65.0 | 5.0 |
| 5.0 | 105.0 | 0.5 |
| 35.0 | 297.5 | 2.5 |

| Detector temperature | 375.0° | C. |
|---|---|---|
| Detector | FID | |
| Make-up gas | He (30.0 mL/min) | |
| Hydrogen flow | 40.0 | mL/min |
| Air | 400.0 | mL/min |

The concentration of linalool is shown as a value in terms of medium amount. A mean value of results obtained from two test tubes is shown in Table 9. No linalool production was observed in the control strain having the introduced control vector pACYC177, whereas the linalool production was confirmed in SWITCH-PphoCΔgcd/AaLINS-ispA* and SWITCH-PphoCΔgcd/CsLINS-ispA* strains.

TABLE 9
Accumulation of linalool when linalool synthase derived
from Actinidia arguta and linalool synthase derived
from Coriandrum sativum were introduced

| | O.D. | Linalool |
|---|---|---|
| Strain | (620 nm) | (mg/L) |
| SWITCH-PphoC Δgcd/pACYC177 | 15.9 | 0.0 |
| SWITCH-PphoC Δgcd/CsLINS-ispA* | 20.2 | 2.6 |
| SWITCH-PphoC Δgcd/AaLINS-ispA* | 12.0 | 1328.2 |

### Example 14

14-1) Acquisition of Limonene Synthase Gene Derived from Picea sitchensis (Sitka Spruce)

A nucleotide sequence (GenBank accession number: DQ195275.1) and an amino acid sequence (GenPept accession number: ABA86248.1.) of a limonene synthase (PsLMS) gene derived from Picea sitchensis have been already known. The amino acid sequence of a limonene synthase protein and the nucleotide sequence of its gene derived from P. sitchensis are shown in SEQ ID NO:90 and SEQ ID NO:91, respectively. In order to efficiently express the PsLMS gene, its secondary structure was resolved and codons were optimized so that the codon usage was same as it in P. ananatis. Moreover, its chloroplast localization signal had been cleaved. An obtained gene was designated as opt_PsLMS. A nucleotide sequence of opt_PsLMS is shown in SEQ ID NO:92. After opt_PsLMS gene was chemically synthesized, cloned into pUC57 (supplied from GenScript) and the resulting plasmid was designated as pUC57-opt_PsLMS.

14-2) Acquisition of Limonene Synthase Gene Derived from Abies grandis (Grand Fir)

A nucleotide sequence (GenBank accession number: AF006193.1) and an amino acid sequence (GenPept accession number: AAB70907.1.) of a limonene synthase (AgLMS) gene derived from Abies grandis have been already known. The amino acid sequence of a limonene synthase protein and the nucleotide sequence of its gene derived from A. grandis are shown in SEQ ID NO:93 and SEQ ID NO:94, respectively. In order to efficiently express the AgLMS gene, its secondary structure was resolved and codons were optimized so that the codon usage was same as it in P. ananatis. Moreover, its chloroplast localization signal had been cleaved. An obtained gene was designated as optAgLMS. A nucleotide sequence of opt_AgLMS is shown in SEQ ID NO:95. After opt_AgLMS gene was chemically synthesized, cloned into pUC57 (supplied from GenScript) and the resulting plasmid was designated as pUC57-opt_AgLMS.

14-3) Acquisition of Limonene Synthase Gene Derived from Mentha spicata (Spearmint)

A nucleotide sequence (GenBank accession number: L13459.1) and an amino acid sequence (GenPept accession number: AAC37366.1.) of a limonene synthase (MsLMS) gene derived from Mentha spicata have been already known. The amino acid sequence of a limonene synthase protein and the nucleotide sequence of its gene derived from M. spicata are shown in SEQ ID NO:96 and SEQ ID NO:97, respectively. In order to efficiently express the MsLMS gene, its secondary structure was resolved and codons were optimized so that the codon usage was same as it in P. ananatis. Moreover, its chloroplast localization signal had been cleaved. An obtained gene was designated as opt_MsLMS. A nucleotide sequence of opt_MsLMS is shown in SEQ ID NO:98. After opt_MsLMS gene was chemically synthesized, cloned into pUC57 (supplied from GenScript) and the resulting plasmid was designated as pUC57-opt_MsLMS.

14-4) Acquisition of Limonene Synthase Gene Derived from Citrus unshiu (Unshu Mikan)

A nucleotide sequence (GenBank accession number: AB110637.1) and an amino acid sequence (GenPept accession number: BAD27257.1) of a limonene synthase (CuLMS) gene derived from Citrus unshiu have been already known. The amino acid sequence of a limonene synthase protein and the nucleotide sequence of its gene derived from C. unshiu are shown in SEQ ID NO:99 and SEQ ID NO:100, respectively. In order to efficiently express the CuLMS gene, its secondary structure was resolved and codons were optimized so that the codon usage was same as it in P. ananatis. Moreover, its chloroplast localization signal had been cleaved. An obtained gene was designated as opt_CuLMS. A nucleotide sequence of opt_CuLMS is shown in SEQ ID NO:101. After opt_CuLMS gene was chemically synthesized, cloned into pUC57 (supplied from GenScript) and the resulting plasmid was designated as pUC57-opt_CuLMS.

14-5) Acquisition of Limonene Synthase Gene Derived from Citrus limon (Lemon)

A nucleotide sequence (GenBank accession number: AF514287.1) and an amino acid sequence (GenPept accession number: AAM53944.1) of a limonene synthase (C1LMS) gene derived from Citrus limon have been already known. The amino acid sequence of a limonene synthase protein and the nucleotide sequence of its gene derived from C. limon are shown in SEQ ID NO:102 and SEQ ID NO:103, respectively. In order to efficiently express the C1LMS gene, its secondary structure was resolved and codons were optimized so that the codon usage was same as it in P. ananatis. Moreover, its chloroplast localization signal had been cleaved. An obtained gene was designated as opt_C1LMS. A nucleotide sequence of opt_C1LMS is shown in SEQ ID NO:104. After opt_C1LMS gene was chemically synthesized, cloned into pUC57 (supplied from GenScript) and the resulting plasmid was designated as pUC57-opt_C1LMS.

14-6) Construction of Co-Expression Plasmid for Opt_PsLMS and ispA* Genes

PCR with pUC57-opt_PsLMS as a template was carried out using primers shown in SEQ ID NO:105 and SEQ ID NO:106. Then, PCR with obtained PCR fragment as a template was carried out using primers shown in SEQ ID NO:107 and SEQ ID NO:106 to obtain a Ptac-opt_PsLMS fragment. Further, PCR with pUC57-ispA* as a template was carried out using primers shown in SEQ ID NO:108 and SEQ ID NO:109 to obtain an ispA* fragment. The purified Ptac-opt_PsLMS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_PsLMS-ispA*.

14-7) Construction of Co-Expression Plasmid for Opt_AgLMS and ispA* Genes

PCR with pUC57-opt_AgLMS as a template was carried out using primers shown in SEQ ID NO:110 and SEQ ID NO:111. Then, PCR with obtained PCR fragment as a template was carried out using primers shown in SEQ ID NO:107 and SEQ ID NO:111 to obtain a Ptac-opt_AgLMS fragment. Further, PCR with pUC57-ispA* as a template was carried out using primers shown in SEQ ID NO:108 and SEQ ID NO:109 to obtain an ispA* fragment. The purified Ptac-opt_AgLMS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_AgLMS-ispA*.

14-8) Construction of Co-Expression Plasmid for Opt_MsLMS and ispA* Genes

PCR with pUC57-opt_MsLMS as a template was carried out using primers shown in SEQ ID NO:112 and SEQ ID NO:113. Then, PCR with obtained PCR fragment as a template was carried out using primers shown in SEQ ID NO:107 and SEQ ID NO:113 to obtain a Ptac-opt_MsLMS fragment. Further, PCR with pUC57-ispA* as a template was carried out using primers shown in SEQ ID NO:108 and SEQ ID NO:109 to obtain an ispA* fragment. The purified Ptac-opt_MsLMS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_MsLMS-ispA*.

14-9) Construction of Co-Expression Plasmid for Opt_CuLMS and ispA* Genes

PCR with pUC57-opt_CuLMS as a template was carried out using primers shown in SEQ ID NO:114 and SEQ ID NO:115. Then, PCR with obtained PCR fragment as a template was carried out using primers shown in SEQ ID NO:107 and SEQ ID NO:115 to obtain a Ptac-opt_CuLMS fragment. Further, PCR with pUC57-ispA* as a template was carried out using primers shown in SEQ ID NO:108 and SEQ ID NO:109 to obtain an ispA* fragment. The purified Ptac-opt_CuLMS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_CuLMS-ispA*.

14-10) Construction of Co-Expression Plasmid for Opt_C1LMS and ispA* Genes

PCR with pUC57-opt_C1LMS as a template was carried out using primers shown in SEQ ID NO:116 and SEQ ID NO:117. Then, PCR with obtained PCR fragment as a template was carried out using primers shown in SEQ ID NO:107 and SEQ ID NO:117 to obtain a Ptac-opt_C1LMS fragment. Further, PCR with pUC57-ispA* as a template was carried out using primers shown in SEQ ID NO:108 and SEQ ID NO:109 to obtain an ispA* fragment. The purified Ptac-opt_C1LMS fragment and ispA* fragment were ligated to pACYC177 (supplied from Nippon Gene) digested with restriction enzymes PstI and ScaI using In-Fusion HD cloning kit (supplied from Clontech) to construct pACYC177-Ptac-opt_C1LMS-ispA*.

### 14-11) Preparation of Limonene Production Strains

Competent cells of SWITCH-PphoCΔgcd were prepared, and pACYC177, pACYC177-Ptac-opt_PsLMS-ispA*, pACYC177-Ptac-opt_AgLMS-ispA*, pACYC177-Ptac-opt_MsLMS-ispA*, pACYC177-Ptac-opt_CuLMS-ispA* or pACYC177-Ptac-opt_C1LMS-ispA* was introduced thereto by electroporation. Resulting strains were designated as SWITCH-PphoCΔgcd/pACYC177, SWITCH-PphoCΔgcd/PsLMS-ispA*, SWITCH-PphoCΔgcd/AgLMS-ispA*, SWITCH-PphoCΔgcd/MsLMS-ispA*, SWITCH-PphoCΔgcd/CuLMS-ispA* and SWITCH-PphoCΔgcd/C1LMS-ispA*.

### Example 15: Evaluation of Ability to Produce Limonene by Limonene Synthase-Expressing Strains Derived from SWITCH-PphoCΔGcd Strain

Microbial cells of SWITCH-PphoCΔgcd/PsLMS-ispA*, SWITCH-PphoCΔgcd/AgLMS-ispA*, SWITCH-PphoCΔgcd/MsLMS-ispA*, SWITCH-PphoCΔgcd/CuLMS-ispA*, SWITCH-PphoCΔgcd/C1LMS-ispA* and SWITCH-PphoCΔgcd/pACYC177 strains obtained in Example 14 in glycerol stock were thawed. Subsequently 10 μL of a microbial cell suspension from each strain was evenly applied onto an LB plate containing 50 mg/L of kanamycin, and cultured at 34° C. for 16 hours. The resulting microbial cells on the plate were picked up in an amount corresponding to about 1 of a loop part of a Nunc disposable 1 μL inoculating loop (supplied from Thermo Fisher Scientific Inc.). The picked up microbial cells were inoculated to 1 mL of limonene fermentation medium described below Table 10 containing 50 mg/L of kanamycin in a head space vial (manufactured by Perkin Elmer, 22 ml CLEAR CRIMP TOP VIAL cat #B0104236), and the vial was tightly sealed with a cap with a butyl rubber septum for the headspace vial (CRIMPS (Cat #B0104240) manufactured by Perkin Elmer). SWITCH-PphoCΔgcd/pACYC177, SWITCH-PphoCΔgcd/PsLMS-ispA*, SWITCH-PphoCΔgcd/AgLMS-ispA* and SWITCH-PphoCΔgcd/MsLMS-ispA* strains were cultured at 30° C. on a reciprocal shaking culture apparatus at 120 rpm for 48 hours. SWITCH-PphoCΔgcd/pACYC177, SWITCH-PphoCΔgcd/CuLMS-ispA* and SWITCH-PphoCΔgcd/C1LMS-ispA* strains were fermented with same manner, cultivation time of these strains were 72 hours.

TABLE 10
Fermentation medium for limonene
production (final concentration)

| Group A | | |
|---|---|---|
| D-Glucose | 4 | g/L |
| MgSO₄•7H₂O | 1 | g/L |

Not adjusted pH, AC 115° C., 10 minutes

| | | |
|---|---|---|
| Group B | | |
| (NH₄)₂SO₄ | 10 | g/L |
| Yeast extract | 50 | mg/L |
| FeSO₄•7H₂O | 5 | mg/L |
| MnSO₄•5H₂O | 5 | mg/L |

Not adjusted pH, AC 115° C., 10 minutes

| | | |
|---|---|---|
| Group C | | |
| MES | 20 | mM |

After adjusting pH to 7.0 with NaOH, sterilized by filtration

After the sterilization, the above Groups A, B and C were mixed.

After completion of cultivation, limonene concentration in the headspace vial was measured by the gas chromatograph mass spectrometer (GCMS-QP2010 manufactured by Shimadzu Corporation) with head space sampler (TurboMatrix 40 manufactured by Perkin Elmer). Detailed analytical condition was shown in below. For GC column, HP-5 ms Ultra Inert (Agilent) was used and limonene standard liquid was prepared with limonene agent (Tokyo Kasei Kogyo).

### Headspace Sampler

Injection time: 0.02 minute

Oven temperature: 80° C.

Needle temperature: 80° C.

Transfer temperature: 80° C.

GC cycle time: 5 minutes

Pressurization time: 3.0 minutes

Pull-up time: 0.2 minutes

Heat retention time: 5 minutes

Carrier gas pressure (high purity helium): 124 kPa

### Gas Chromatography Part

### Carrier gas: He

| | |
|---|---|
| Temperature in vaporization room | 200.0° C. |
| Temperature condition | 175° C. (constant temperature) |

Mass spectrometry part

| Temperature in ion source | 250° | C. |
|---|---|---|
| Temperature in interface | 250° | C. |
| Electric voltage for detector | 0.1 | kV |
| Detection ion molecular weight | 68 | |
| Auxiliary ion molecular weight | 93 | |
| Filament lighting time | 2.0-3.5 | min |

The concentration of limonene is shown as a value in terms of medium amount. A mean value of results obtained from two vials is shown in Tables 11 and 12. No limonene production was observed in the control strain having the introduced control vector pACYC177, whereas the limonene production was confirmed in SWITCH-PphoCΔgcd/PsLMS-ispA*, SWITCH-PphoCΔgcd/AgLMS-ispA*, SWITCH-PphoCΔgcd/MsLMS-ispA*, SWITCH-PphoCΔgcd/CuLMS-ispA*, and SWITCH-PphoCΔgcd/C1LMS-ispA* strains.

TABLE 11
Accumulation of limonene when limonene synthase derived
from Picea sitchensis, Abies grandis and Mentha spicata were introduced

| | O.D. | Limonene |
|---|---|---|
| Strain | (600 nm) | (mg/L) |
| SWITCH-PphoC Δgcd/pACYC177 | 2.6 | 0.0 |
| SWITCH-PphoC Δgcd/PsLMS-ispA* | 1.6 | 0.3 |
| SWITCH-PphoC Δgcd/AgLMS-ispA* | 1.6 | 121 |
| SWITCH-PphoC Δgcd/MsLMS-ispA* | 1.6 | 117 |

TABLE 12
Accumulation of limonene when limonene synthase derived
from Citrus unshiu and Citrus limon were introduced

| | O.D. | Limonene |
|---|---|---|
| Strain | (600 nm) | (mg/L) |
| SWITCH-PphoC Δgcd/pACYC177 | 2.2 | 0.0 |
| SWITCH-PphoC Δgcd/CuLMS-ispA* | 1.5 | 33 |
| SWITCH-PphoC Δgcd/ClLMS-ispA* | 1.1 | 129 |

### INDUSTRIAL APPLICABILITY

The method of the present invention is useful for the production of an isoprene monomer and an isoprene polymer.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1-32. (canceled)

33. A method of producing an isoprenoid compound, comprising:
(1) culturing an isoprenoid compound-forming microorganism in the presence of a growth promoting agent at a sufficient concentration to grow the isoprenoid compound-forming microorganism;
(2) decreasing the sufficient concentration of the growth promoting agent to induce formation of the isoprenoid compound by the isoprenoid compound-forming microorganism; and
(3) culturing the isoprenoid compound-forming microorganism to form the isoprenoid compound,
wherein the isoprenoid compound-forming microorganism has an ability to form the isoprenoid compound depending on a promoter which is inversely dependent on the growth promoting agent,
wherein the isoprenoid compound-forming microorganism is an isoprenoid compound-forming bacterium, and
wherein the growth promoting agent is a phosphorus compound.

34. The method according to claim 33, wherein the isoprenoid compound-forming microorganism includes a gene encoding an isoprenoid compound-synthetic enzyme which is placed under the control of the promoter.

35. The method according to claim 33, wherein the isoprenoid compound-forming microorganism has an enhanced methylerythritol phosphate pathway and/or an enhanced mevalonate pathway.

36. The method according to claim 33, wherein the isoprenoid compound is a monoterpene, and the isoprenoid compound-forming microorganism is a monoterpene-forming microorganism.

37. The method according to claim 33, wherein the isoprenoid compound is a limonene, and the isoprenoid compound-forming microorganism is a limonene-forming microorganism.

38. The method according to claim 33, wherein the isoprenoid compound is a linanol, and the isoprenoid compound-forming microorganism is a linanol-forming microorganism.

39. The method according to claim 33, wherein the isoprenoid compound is an isoprene, and the isoprenoid compound-forming microorganism is an isoprene-forming microorganism.

40. The method according to claim 33, wherein the promoter is a phosphorus deficiency-inducible promoter.

41. The method according to claim 40, wherein the phosphorus deficiency-inducible promoter is a promoter of a gene encoding an acid phosphatase, or a promoter of a gene encoding a phosphorus uptake carrier.

42. The method according to claim 33, wherein formation of the isoprenoid by the isoprenoid-forming microorganism is induced in the presence of a total phosphorus at concentration of 50 mg/L or less.

43. The method according to claim 33, wherein the isoprenoid-forming rate in an induction phase is 600 ppm/vvm/h or less.

44. The method according to claim 33, wherein the isoprenoid compound-forming microorganism has an ability to synthesize dimethylallyl diphosphate via a methylerythritol phosphate pathway.

45. The method according to claim 33, wherein the isoprenoid compound-forming microorganism has an ability to synthesize dimethylallyl diphosphate via a mevalonate pathway.

46. The method according to claim 33, wherein the isoprenoid compound-forming microorganism is a microorganism belonging to the family Enterobacteriaceae.

47. The method according to claim 33, wherein the isoprenoid compound-forming microorganism is a microorganism belonging to the genus Pantoea, Enterobacter, or Escherichia.

48. The method according to claim 33, wherein the isoprenoid compound-forming microorganism is Pantoea ananatis, Enterobacter aerogenes or Escherichia coli.

49. A method of producing an isoprene-containing polymer, comprising:
(I) forming a monomer composition comprising isoprene monomer produced by the method according to claim 1; and
(II) polymerizing the monomer composition to form the isoprene-containing polymer.

50. A method for producing a rubber composition, comprising:
(A) preparing an isoprene-containing polymer by a method according to claim 49; and
(B) mixing the isoprene-containing polymer with one or more rubber composition components.

51. A method for producing a tire, comprising:
(i) producing a rubber composition by the method of claim 50; and
(ii) applying the rubber composition to manufacture a tire.


SEQUENCE LISTING (117 sequences)

SEQ ID NO: 1 - DNA, 1783 bp, Enterobacter aerogenes
ataatgtggtgctgctgcaaaccatgcgcggtttcttcgagcttctgcaatcctcggtgaagcagagccgccagcgcatgtatctggtgccgccggtgttcgcgcgtctgaccgaacaacatcaggcggtaatggaagcgatcttcgccggcgacgcggaaggcgcccgccaggcgatgatggcccacctcggttttgtccacgccaccattaaacgttttgatgaagaccaggcccgccaggcgcgtattacccgcctgcccggtgaccacaatgaaaattccagggagaactcgtgatgattatttccgccgccagcgactatcgcgccgccgcacagcgtatcctgccaccatttctgttccactatatcgacggcggcgcctacgccgaacataccctgcgccgcaacgttgaagacctctccgacgtggcgctccgccagcgcattctgcgcaatatgtcggatctgagcctggagaccacgttattcaacgaaaagctggcgatgccaacggcgctggcgccggtcgggctgtgcggcatgtacgcgcgccgcggcgaagtgcaggcagcgggcgcggcggacgacaaaggcatcccgtttaccctctctaccgtttccgtttgcccgattgaagaggtcgccccgaccatcaaacgcccgatgtggttccagctgtatgtcctgcgcgatcgcggatttatgcgcaatgcactggagcgcgccaaagccgcaggctgctccacgctggtctttaccgtggatatgccgacccccggcgcgcgctatcgcgacgcccattctggcatgagcggcccaaacgccgccatgcgccgctactggcaggccgtcactcatccgcagtgggcatgggacgtcggtctcaacggccgcccgcacgacctgggcaatatctccgcctacctcggcaaaccgaccgggcttgaggattacatcggctggctggcgaataatttcgatccgtcgatttcatggaaagacctggagtggatccgcgacttctgggacggcccaatggtgatcaaagggatcctcgacccggaggacgcccgcgatgcggtgcgcttcggcgctgacggcattgtcgtctccaaccacggcggccgccagcttgacggcgtgctctcttccgcccgcgccctgcccgccattgccgatgcggttaaaggcgacatcactattctcgccgacagcggtatccgcaacggtctcgacgtggtgcggatgatcgccctgggcgccgacagcgtgctgctgggccgcgcgtatctgtacgcgctggcgacccacggcaagcagggggtggcgaatctgctcaaccttatcgagaaagagatgaaagtggcgatgacgctgaccggggcgaaatccatcagcgaaatcagccgcgattcgctggtgcagaatgccgaagcgctgcagacctttgatgcgctcaagcagggtaacgccgcgtaattccccgcgttttcccttacgccgcccctctccctcagggtgaggggcccgtccggctggaatcggttttctccctctccctctgggagagggtcggggtgagggttcaacaaatatctgctatcctgccccccgctattaagggggcagtatgctgaacatcgtgttattcgaaccagaaatcccgccgaacaccggcaatatcatccgcctgtgcgccaacaccggcttcaatttacatatcattgagccaatgggctttacctgggacgataaacgcctgagacgcgccgggttgga

SEQ ID NO: 2 - DNA, 89 bp, Artificial Sequence
  Primer for amplifying lambda attL-TetR- lambda attR-Ptac-KDyI
ccacgcacggattacccgcctgcccggtgagcataatgagcattcgagggagaaaaacgctgaagcctgcttttttatactaagttggc

SEQ ID NO: 3 - DNA, 90 bp, Artificial Sequence
  Primer for amplifying lambda attL-TetR- lambda attR-Ptac-KDyI
acagcgccgaacggtcccctctccctgagggagagggttagggtgagggggcgcaaacgattatagcattctatgaatttgcctgtcatt

SEQ ID NO: 4 - DNA, 26 bp, Artificial Sequence
  Primer for confirming delta lld:: lambda attL- TetR-lambda attR-Ptac-KDyI
catgaaacgattcgatgaagatcagg

SEQ ID NO: 5 - DNA, 26 bp, Artificial Sequence
  Primer for confirming delta lld:: lambda attL- TetR-lambda attR-Ptac-KDyI
ctggttcgtaaagtacgatgtttagc

SEQ ID NO: 6 - DNA, 2319 bp, Enterobacter aerogenes
gcatgccgctgatgccatccacgccgtcgattagcttatgtagaccgtgaaacaacattaagccgccaacggcaagtcgtaacaagagtttaccggcatcttcatgcgacagcgttttatttactgcgtttaacaatcccttaaccatttgatgtgcttcctgttttacaccctgtgtgatcccagagtatgcacaatattccaccaacaaaaccagtgaaaaataaggggcaattggcaacgtaacctgtagtttcatctaagcttgatagcgttatcacaaaaaggagatggaaaaccatgaaacagaccgtagcgtcatatattgccaaaacgcttgaacaggccggcgtgaaacgtatttggggagtgaccggggattccctcaacggcttgagcgacagtttaaaccgcatggggaccatcgaatggatgccgacccgccatgaagaggttgccgcctttgccgccggtgccgaagcccaactcaccggcgagctggcggtgtgcgccggttcctgcggaccgggcaaccttcatctgattaacggtttgtttgattgccatcgtaaccacgtaccggttctggctatcgccgcccatattccgtcgagcgaaatcggcagcggttatttccaggaaacgcatccgcaggagctgttccgcgaatgtagccactactgcgagctggtctccacaccggagcaaattccgcaagttctggctatcgccatgcgtaaggcggtgattaaccgcggcgtgtctgtggtggtcctgcccggcgacgtggcgctgaaaccggccccggaaagcgccagcagccactggtatcacgctccgcagcccacggtcactccggcggatgaagagctgcacaagctggcgcagctgatccgctactcgagtaatattgccctgatgtgcggcagcggctgcgccggcgcgcaccaggagctggtggagtttgcggcaaaaattaaggcccctgtcgtccatgccctgcgcgggaaagagcacgtcgaatacgacaatccttacgatgttggcatgaccgggcttatcggcttctcttccggtttccataccatgatgaacgccgacacgctgatcctgctcggcacccagttcccctatcgcgcgttctaccctaccgacgccaaaattattcagatcgacatcaaccccggtagcatcggcgcgcatagtaaggtcgacatggcgctggtgggcgatatcaaatcgacactgaaagccctgctgccgctgctggaagagaaaaccgatcgtcgcttcctcgataaagcgctggaggattatcgcgaagcgcgtaaagggcttgacgatctcgccaaacccagcgaaaaagcgctccatccccagtatctggcgcagcagattagccgctttgccgacgacgacgcgatctttacctgcgacgtcggtacccccaccgtctgggcggcacgctatctgcagatgaacggcaaacgccgcctgttgggttcgtttaaccacggctcgatggccaacgccatgccgcaggctattggcgccaaagcgaccgcgccgaatcgccaggtcatcgccatgtgcggcgatggcggtttcagcatgctgatgggggattttctatcgctggcgcagatgaagctgccggtgaaaatcgttatttttaataacagcattctcggctttgtagcgatggagatgaaagccggcggctatctgaccgacggtaccgagttgcacgacaccaatttcgcccgcattgccgaagcctgcgggatcaaaggaatacgcgtcgagaaagcttctgaagttgatgaggcgctgcagaccgcctttagcaccgacggtccggtattagtcgatgtagtggtagcgaaggaagagctggcgatcccgccgcaaattaagctggagcaggccaaaggatttagcctgtatatgctgcgggcgattatcagcggacgcggcgatgaagtgatcgaactggcgaaaaccaactggctcaggtaaaacaatgctatcaccctgaatcagaacaaggatatgccgtgatagatcttcgcagtgataccgtaactcgccccgggcgcgccatgatggaggccatgatggccgccccggtcggggacgatgtgtatggcgacgacccaaccgtcaatgaactccagcgctatgccgctgagctcgcaggcaaagaggcggcgctgtttctgcctaccggcacccaggccaatctcgttggcctgcttagccactgccagcgcggcgaagagtacatcgtcggccagggcgcgcataactatctgtatg

SEQ ID NO: 7 - DNA, 90 bp, Artificial Sequence
  Primer for amplifying PMK gene from E.coli MG1655 Ptac-KDyI strain
tatcactgcgaagatctatcacggcatatccttgttctgattcagggtgatagcattgttttatttatcaagataagtttccggatcttt

SEQ ID NO: 8 - DNA, 56 bp, Artificial Sequence
  Primer for amplifying PMK gene from E.coli MG1655 Ptac-KDyI strain
cggataacaatttcacacaaggagactgccatgtcagagttgagagccttcagtgc

SEQ ID NO: 9 - DNA, 89 bp, Artificial Sequence
  Primer for amplifying lambda attL-Kmr- lambda attR-Ptac cassette
acgtaacctgtagtttcatctaagcttgatagcgttatcacaaaaaggagatggaaaacctgaagcctgcttttttatactaagttggc

SEQ ID NO: 10 - DNA, 56 bp, Artificial Sequence
  Primer for amplifying lambda attL-Kmr- lambda attR-Ptac cassette
gcactgaaggctctcaactctgacatggcagtctccttgtgtgaaattgttatccg

SEQ ID NO: 11 - DNA, 22 bp, Artificial Sequence
  Primer for confirming delta poxB:: lambda attL- Kmr-lambda attR-Ptac-PMK
gtcgattagcttatgtagaccg

SEQ ID NO: 12 - DNA, 21 bp, Artificial Sequence
  Primer for confirming delta poxB:: lambda attL- Kmr-lambda attR-Ptac-PMK
tggagttcattgacggttggg

SEQ ID NO: 13 - DNA, 2883 bp, Enterobacter aerogenes
gccagcggctttgaacacagcatcgctaatatgtttatgatcccgatgggtatcgtaattcgcaactttgcaagcccggaattctggaccgctatcggttcaactccggagagtttttctcacttaaccgttatgaacttcatcactgataacctgattccagtcactatcggaaacatcatcggcggtggtttgttggttgggttgacatactgggtcatttacctgcgtggcgacgatcatcattaatggttgtctcaggcagtaaataaaaaatccacttaagaaggtaggtgttacatgtccgagcttaatgaaaagttagccacagcctgggaaggttttgcgaaaggtgactggcagaacgaagtcaacgtacgtgactttatccagaaaaactacaccccatatgaaggtgacgaatccttcctggctggcgcaactgatgcgaccaccaagctgtgggacagcgtaatggaaggcgttaaacaggaaaaccgcactcacgcgcctgttgatttcgacacttccctcgcatccaccatcacttctcacgacgcgggctacatcgagaaagcgctcgagaaaatcgttggtctgcaaactgaagccccgctgaaacgtgcgattatcccgttcggcggtatcaaaatggttgaaggttcctgcaaagcgtacaatcgcgaactggacccgatgctgaaaaaaatcttcaccgagtaccgtaaaactcacaaccagggcgttttcgacgtatataccccggacatcctgcgctgccgtaaatccggcgtactgaccggtctgccggatgcttacggccgtggtcgtatcatcggtgactatcgtcgcgttgcgctgtacggtatcgacttcctgatgaaagacaaattcgcccagttcaactcgctgcaggcgaaactggaaagcggcgaagatctggaagcaaccatccgtctgcgtgaagaaattgctgaacagcatcgcgcgctgggtcagatcaaagagatggcggctaaatatggctacgacatctccggtccggcgactaccgctcaggaagctattcagtggacctacttcggttacctggccgccgttaaatctcagaacggcgcggcaatgtccttcggtcgtacttccagcttcctggatatctacatcgaacgtgacctgcaggcgggtaaaatcaccgagcaagacgcgcaggaaatggttgaccacctggtcatgaaactgcgtatggttcgcttcctgcgtaccccggaatatgatgaactgttctccggcgacccgatttgggcaacggaatctatcggtggtatgggcgttgacggccgtactctggtaaccaaaaacagcttccgcttcctgaacaccctgtacaccatggggccgtctccggagccgaacatcactatcctgtggtctgaaaaactgccgctgagctttaagaaattcgccgctaaagtatccatcgatacctcttctctgcagtacgagaacgatgacctgatgcgcccggacttcaacaacgacgattacgctatcgcatgctgcgtaagcccgatgattgttggtaaacaaatgcagttcttcggcgctcgcgctaacctcgcgaaaaccatgctgtatgctatcaacggcggcgttgatgaaaaactgaaaatgcaggttggtccgaaatctgaaccgatcaaaggcgacgtcctgaacttcgacgaagttatggagcgcatggatcacttcatggactggctggctaaacagtacgtcaccgcgctgaacatcattcattacatgcatgacaagtacagctacgaagcctctctgatggcgctgcacgaccgtgacgttatccgcaccatggcgtgtggtatcgcaggtctgtccgttgctgctgactccctgtctgctatcaaatatgcgaaagttaaaccgattcgtgacgaagacggtctggctgttgacttcgaaatcgaaggcgaatacccgcagtttggtaacaacgatgctcgcgtcgatgacatggccgttgacctggttgaacgtttcatgaagaaaattcagaaactgcacacctaccgcaacgctatcccgactcagtccgttctgaccatcacttctaacgtcgtgtatggtaagaaaaccggtaacaccccagatggtcgtcgcgctggcgcgccgttcggaccaggtgctaacccgatgcacggccgtgaccagaaaggtgctgtagcctctctgacttccgttgctaaactgccgtttgcttacgctaaagatggtatctcttacaccttctctatcgtgccgaacgcgctgggtaaagatgacgaagttcgtaagaccaacctggcgggcctgatggatggttacttccaccacgaagcgtccatcgaaggtggtcagcacctgaacgtgaacgtcatgaaccgcgaaatgctgctcgacgcgatggaaaacccggaaaaatatccgcagctgaccattcgtgtatctggctacgcggtacgttttaactccctgactaaagaacagcagcaggatgttattacccgtaccttcactcagaccatgtaattccctgtctgactgaaaaagcgtacaataaaggccccacatcagtggggcctttttaacacgtgattccctgccccagcctgctttgccagttatctatactttgggtacctgtcaaaacagacttaacacagccggtttgagctgtgcatcacaggccctggagggccgaacccggagatatcaccgcaatgtcaactattggtcgtattcactcctttgaatcctgtggcaccgtcgatggcccaggcatccgctttattaccttcttccagggctgcctgatgcgctgcctttact

SEQ ID NO: 14 - DNA, 60 bp, Artificial Sequence
  Primer for amplifying MVD gene from E.coli MG1655 Ptac-KDyI strain
cggataacaatttcacacaaggagactgccatgaccgtttacacagcatccgttaccgca

SEQ ID NO: 15 - DNA, 90 bp, Artificial Sequence
  Primer for amplifying MVD gene from E.coli MG1655 Ptac-KDyI strain
gttaaaaaggccccactgatgtggggcctttattgtacgctttttcagtcagacagggaattattcctttggtagaccagtctttgcgtc

SEQ ID NO: 16 - DNA, 89 bp, Artificial Sequence
  Primer for amplifying lambda attL-Kmr- lambda attR-Ptac cassett
catcattaatggttgtctcaggcagtaaataaaaaatccacttaagaaggtaggtgttactgaagcctgcttttttatactaagttggc

SEQ ID NO: 17 - DNA, 60 bp, Artificial Sequence
  Primer for amplifying lambda attL-Kmr- lambda attR-Ptac cassette
tgcggtaacggatgctgtgtaaacggtcatggcagtctccttgtgtgaaattgttatccg

SEQ ID NO: 18 - DNA, 20 bp, Artificial Sequence
  Primer for amplifying delta pflB:: lambda attL- Kmr-lambda attR-Ptac-MVD
ggctttgaacacagcatcgc

SEQ ID NO: 19 - DNA, 18 bp, Artificial Sequence
  Primer for amplifying delta pflB:: lambda attL- Kmr-lambda attR-Ptac-MVD
ctgttttgacaggtaccc

SEQ ID NO: 20 - DNA, 1341 bp, Enterobacter aerogenes
atatccgcagctgaccattcgtgtatctggctacgcggtacgttttaactccctgactaaagaacagcagcaggatgttattacccgtaccttcactcagaccatgtaattccctgtctgactgaaaaagcgtacaataaaggccccacatcagtggggcctttttaacacgtgattccctgccccagcctgctttgccagttatctatactttgggtacctgtcaaaacagacttaacacagccggtttgagctgtgcatcacaggccctggagggccgaacccggagatatcaccgcaatgtcaactattggtcgtattcactcctttgaatcctgtggcaccgtcgatggcccaggcatccgctttattaccttcttccagggctgcctgatgcgctgcctttactgccataaccgcgacacctgggatacccatggcggtaaagaaatcaccgttgaagaattgatgaaagaagtggtgacctatcgccactttatgaatgcctcgggcggcggcgtcaccgcctccggcggcgaggcgatcctgcaggctgaatttgtccgcgactggttccgcgcctgtaaaaaagaaggtatccacacctgtctggataccaacggcttcgtacgccgttacgatccggtgattgacgagctgctggaagtcaccgatctggtgatgcttgacctcaagcagatgaacgatgaaattcaccagaacctggtcggcgtttctaaccaccgtacgctggaattcgcccagtatttgtcgaagaaagggattaacgtgtggatccgctacgtggtggttcccggctggtctgatgatgacgattccgcacatcgtctgggtgagtttacccgcgatatgggtaacgtcgagaaaatcgaactcctgccctaccatgagctgggtaaacacaaatgggtggcaatgggcgaagagtacaaacttgacggcgtacacccaccgaagaaaaagaccatggagcgggtaaaaggcatcctggagcaatatggccataaggtgatgtactaaaccggcagcgggccggaggtactctcaccacggcccgcaactataatagtatcaatcccgccgataacgcctcatcatgagcgcgataccgtctgcccgcagaaatattcattaatcaaaacggactacgcgcggccttcgccgcgcgccagattggttacgcgtgtgccaccggcgtcggatgatgcccggctttgcgcagcagggtcagcagatagataaacgatacgctggcgatcatgataaacagcagattatccgagaagttctgcatcaacatcgccgtcagggtcggcccca

SEQ ID NO: 21 - DNA, 81 bp, Artificial Sequence
  Primer for amplifying yIDI gene from E. coli MG1655 Ptac-KDyI strain
cggataacaatttcacacaaggagactgccatgaccgcggataacaacagcatgccccatggtgcagtatctagttacgcc

SEQ ID NO: 22 - DNA, 90 bp, Artificial Sequence
  Primer for amplifying yIDI gene from E. coli MG1655 Ptac-KDyI strai
cgggattgatactattatagttgcgggccgtggtgagagtacctccggcccgctgccggtttatagcattctatgaatttgcctgtcatt

SEQ ID NO: 23 - DNA, 89 bp, Artificial Sequence
  Primer for confirming lambda attL-Kmr-lambda attR-Ptac cassette
cagccggtttgagctgtgcatcacaggccctggagggccgaacccggagatatcaccgcatgaagcctgcttttttatactaagttggc

SEQ ID NO: 24 - DNA, 81 bp, Artificial Sequence
  Primer for confirming lambda attL-Kmr-lambda attR-Ptac cassett
ggcgtaactagatactgcaccatggggcatgctgttgttatccgcggtcatggcagtctccttgtgtgaaattgttatccg

SEQ ID NO: 25 - DNA, 22 bp, Artificial Sequence
  Primer for confirming delta pflA:: lambda attL- Kmr-lambda attR-Ptac-yID
cagttatctatactttgggtac

SEQ ID NO: 26 - DNA, 23 bp, Artificial Sequence
  Primer for confirming delta pflA:: lambda attL- Kmr-lambda attR-Ptac-yIDI
tttgattaatgaatatttctgcg

SEQ ID NO: 27 - DNA, 30 bp, Artificial Sequence
  Primer for amplifying DNA fragment containing promoter region of bud operon and ORF region of BudR
gagccacttcctcgttcaacaaatataaga

SEQ ID NO: 28 - DNA, 30 bp, Artificial Sequence
  Primer for amplifying DNA fragment containing promoter region of bud operon and ORF region of BudR
ttagaacatctctaaaaatcgcttcaccgt

SEQ ID NO: 29 - DNA, 979 bp, Artificial Sequence
  DNA fragment containing promoter region of bud operon (1st to 106th positions) and ORF region of BudR (107th to 979th positions)
gagccacttcctcgttcaacaaatataagaaaggttaaataaaggttgacccgattcagctcacagttccaatatagaaaccatgctggtttgagacgttttcgatatggaacttcgttatctacgctattttgtcgccgtcgcccggacgcggcacttcacccgggcagcgaaagaactgggtatctcgcagccaccgttaagtcagcaaattcagcggcttgagcgagaggttggcactccgctgttgcgtcggctaacccgcggggtggagctgaccgaagccggggagtccttttatgaagatgcctgccaaatcctcgcgctgagcgatgcggcgctggaaaaggccaagggcattgcccgcggaatgaacggcagcctgtcgttaggcattaccagttctgatgctttccatccacaaatcttcaccttgctgcaccgttttcagctcgatcacccaggcgtcgtcctccatcagcgggagggcaacatggcaaatttgatggcggcgctgagcgaggcggagatcgatatcgcctttgtccgattgccgtgtgaaagcagtaaggcgtttaacttgcgtatcattgatgaagagccaatggtcattgcgctgccgcgcgataatccattgtcagcggaaccgacgctggcgctggaacagttgcgggacgtcgcgccgatcctcttcccgcgcgaagtggcgccgggtttgtatgagctggtgttcaatagctgcctgcgcgccggtatcgatatgaaccgcgccaggcagtcgtcgcagatttcatcgtcgctgagtatggttaacgccggatttggtttcgcgctggtgccgcagtcgatgacctgtatcgcgctgcccaacgtcagctatcaatcgatacaggggacgccggtcaagaccgatattgccatcgcctggcggcgttttgagcgctcgcgcacggtgaagcgatttttagagatgttctaa

SEQ ID NO: 30 - DNA, 45 bp, Artificial Sequence
  Primer for amplifying DNA fragment of pMW-Para- mvaES-Ttrp in which arabinose promoter is deleted
acgaggaagtggctcatgaaaaccgtggttattatcgatgcgctg

SEQ ID NO: 31 - DNA, 45 bp, Artificial Sequence
  Primer for amplifying DNA fragment of pMW-Para- mvaES-Ttrp in which arabinose promoter is deleted
ttagagatgttctaaactggccgtcgttttacaacgtcgtgactg

SEQ ID NO: 32 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 33 - DNA, 2412 bp, Enterococcus faecalis
atgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataa

SEQ ID NO: 34 - DNA, 2412 bp, Artificial Sequence
  DNA having modified codons, which encodes mvaE derived from Enterococcus faecalis
atgaaaaccgtggttattatcgatgcgctgcgcacgccgattggtaaatataaaggcagcctgtctcaagtgagcgccgttgatctgggtacgcatgtgaccacgcagctgctgaaacgtcacagcaccatctctgaagaaattgatcaggtgatctttggtaacgttctgcaagccggtaatggtcagaatccggcacgtcagattgcaatcaacagtggcctgagccatgaaattccggcgatgaccgtgaatgaagtttgcggtagcggcatgaaagcggttattctggccaaacagctgatccagctgggtgaagcggaagtgctgattgccggcggtatcgaaaacatgagtcaggcaccgaaactgcaacgttttaattatgaaaccgaaagctacgatgccccgttcagctctatgatgtatgatggcctgaccgatgcatttagcggtcaggcgatgggcctgacggcagaaaacgtggcggaaaaataccatgttacccgcgaagaacaggatcagttttctgttcacagtcagctgaaagcggcccaggcccaggcagaaggtattttcgccgatgaaatcgcaccgctggaagtgtctggtacgctggttgaaaaagatgaaggcattcgtccgaatagtagcgtggaaaaactgggcaccctgaaaacggtgttcaaagaagatggcaccgttacggcgggcaatgcaagcaccatcaatgatggtgcgagtgccctgattatcgcgagccaggaatatgcagaagcgcatggcctgccgtacctggccattatccgcgattctgtggaagttggtattgatccggcatatatgggcattagtccgatcaaagcgattcagaaactgctggcccgtaaccagctgaccaccgaagaaattgatctgtacgaaatcaatgaagcgtttgcagcgaccagtattgtggttcagcgcgaactggccctgccggaagaaaaagttaacatttatggcggtggcatcagcctgggtcacgcaattggtgccaccggtgcacgtctgctgaccagtctgagctatcagctgaatcagaaagagaaaaaatacggtgtggcaagcctgtgtattggtggcggtctgggtctggccatgctgctggaacgtccgcagcagaagaaaaactctcgtttttaccagatgagtccggaagaacgtctggccagtctgctgaacgaaggccagattagcgcagataccaaaaaagaattcgaaaatacggcactgtctagtcagatcgcgaaccatatgattgaaaatcagatcagcgaaaccgaagtgccgatgggtgttggcctgcacctgaccgtggatgaaacggattatctggttccgatggcgacggaagaaccgagcgttattgccgcactgtctaacggtgcaaaaatcgcgcagggctttaaaaccgtgaatcagcagcgtctgatgcgcggccagattgtgttctacgatgttgcggatccggaaagcctgatcgataaactgcaagtgcgcgaagccgaagtttttcagcaggcagaactgagctatccgtctattgtgaaacgtggcggtggcctgcgcgatctgcaataccgtacctttgatgaaagtttcgtgagcgttgatttcctggtggatgttaaagatgccatgggtgcaaacatcgtgaatgcgatgctggaaggcgttgccgaactgtttcgtgaatggttcgcggaacagaaaatcctgttttctatcctgagtaactacgcgaccgaaagcgtggttaccatgaaaacggccattcctgtgagccgcctgtctaaaggtagtaatggccgtgaaattgcggaaaaaatcgttctggcgagccgctatgcctctctggatccgtaccgtgccgtgacccataacaaaggtattatgaatggcatcgaagcagtggttctggcgaccggtaacgatacccgtgccgtgtctgcaagttgccatgcattcgcagttaaagaaggtcgttatcagggcctgaccagctggacgctggatggtgaacagctgatcggcgaaatttctgtgccgctggccctggcaaccgtgggtggcgcgacgaaagttctgccgaaaagccaggcggccgcagatctgctggcggtgaccgatgcaaaagaactgtctcgcgtggttgcggccgttggtctggcacagaatctggcagcgctgcgtgcgctggtgtctgaaggtattcagaaaggccacatggcactgcaagcccgtagtctggccatgaccgtgggtgcaacgggcaaagaagtggaagcagttgcgcagcagctgaaacgccagaaaaccatgaaccaggatcgtgccatggcaatcctgaatgatctgcgcaaacagtaa

SEQ ID NO: 35 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 36 - DNA, 1152 bp, Enterococcus faecalis
atgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaa

SEQ ID NO: 37 - DNA, 1152 bp, Artificial Sequence
  DNA having modified codons, which encodes mvaS derived from Enterococcus faecalis
atgaccattggtatcgataaaattagctttttcgtgccgccgtattacatcgatatgacggcgctggccgaagcacgtaacgttgatccgggcaaatttcatattggcatcggtcaggatcagatggcggtgaacccgatttctcaggatatcgttaccttcgcggccaatgcagcggaagcaattctgacgaaagaagataaagaagcgattgatatggtgatcgttggcaccgaaagctctatcgatgaaagtaaagccgcagcggtggttctgcaccgtctgatgggcattcagccgtttgcgcgcagcttcgaaatcaaagaagcctgctatggcgcgaccgccggtctgcaactggccaaaaaccatgtggcactgcacccggataaaaaagttctggtggttgccgcagatattgcgaaatacggtctgaatagcggcggtgaaccgacccagggtgcaggtgccgtggcaatgctggttgcatctgaaccgcgtattctggcgctgaaagaagataacgtgatgctgacccaggatatctatgatttttggcgtccgaccggtcatccgtacccgatggtggatggcccgctgagtaatgaaacctatattcagagcttcgcccaggtttgggatgaacataaaaaacgtacgggtctggattttgcggattatgatgcactggcgttccacattccgtacaccaaaatgggcaaaaaagcgctgctggccaaaatcagcgatcagacggaagccgaacaggaacgtattctggcacgctatgaagaaagcatcgtgtactctcgtcgcgttggcaacctgtataccggttctctgtacctgggcctgattagtctgctggaaaacgcgaccacgctgacggccggcaatcagatcggtctgttttcttatggcagtggtgccgtggcagaatttttcaccggtgaactggttgccggctaccagaaccatctgcaaaaagaaacccacctggccctgctggataatcgcacggaactgtctattgcagaatatgaagcaatgtttgcggaaaccctggatacggatatcgatcagaccctggaagatgaactgaaatatagtattagcgcgatcaacaatacggtgcgtagttaccgcaattaa

SEQ ID NO: 38 - DNA, 40 bp, Artificial Sequence
  Primer for amplifying a fragment comprising Para composed of araC and ara BAD promoters from E.coli
tgaattcgagctcggtacccactcttcctttttcaatatt

SEQ ID NO: 39 - DNA, 40 bp, Artificial Sequence
  Primer for amplifying a fragment comprising Para composed of araC and ara BAD promoters from E.coli
ataataaccacggttttcattttttataacctccttagag

SEQ ID NO: 40 - DNA, 40 bp, Artificial Sequence
  Primer for amplifying a fragment comprising EFmvaE gene
ctctaaggaggttataaaaaatgaaaaccgtggttattat

SEQ ID NO: 41 - DNA, 55 bp, Artificial Sequence
  Primer for amplifying a fragment comprising EFmvaE gene
ttatcgataccaatggtcatgtttttttacctcctttactgtttgcgcagatcat

SEQ ID NO: 42 - DNA, 55 bp, Artificial Sequence
  Primer for amplifying a fragment comprising EFmvaS gene
atgatctgcgcaaacagtaaaggaggtaaaaaaacatgaccattggtatcgataa

SEQ ID NO: 43 - DNA, 40 bp, Artificial Sequence
  Primer for amplifying a fragment comprising EFmvaS gene
cagcggaactggcggctcccttaattgcggtaactacgca

SEQ ID NO: 44 - DNA, 40 bp, Artificial Sequence
  Primer for amplifying a fragment comprising Ttrp
tgcgtagttaccgcaattaagggagccgccagttccgctg

SEQ ID NO: 45 - DNA, 38 bp, Artificial Sequence
  Primer for amplifying a fragment comprising Ttrp
gtcgactctagaggatccctaatgagaattagtcaaat

SEQ ID NO: 46 - DNA, 42 bp, Artificial Sequence
  Primer (Linker-F)
agctttagggataacagggtaatctcgagctgcaggcatgca

SEQ ID NO: 47 - DNA, 42 bp, Artificial Sequence
  Primer (Linker-R)
agcttgcatgcctgcagctcgagattaccctgttatccctaa

SEQ ID NO: 48 - DNA, 55 bp, Artificial Sequence
  Primer (lldD5′ CAS)
tttttaagctttagggataacagggtaatctcgagatttaaagcggctgctttac

SEQ ID NO: 49 - DNA, 43 bp, Artificial Sequence
  Primer (lldD3′ CAS)
tttttaagcttgcatgcctgcagtatttaatagaatcaggtag

SEQ ID NO: 50 - DNA, 54 bp, Artificial Sequence
  Primer (phoC5′ CAS)
tttttaagctttagggataacagggtaatctcgagtggataacctcatgtaaac

SEQ ID NO: 51 - DNA, 44 bp, Artificial Sequence
  Primer (phoC3′ CAS)
tttttaagcttgcatgcctgcagttgatgtctgattatctctga

SEQ ID NO: 52 - DNA, 54 bp, Artificial Sequence
  Primer (pstS5′ CAS)
tttttaagctttagggataacagggtaatctcgagagcctctcacgcgtgaatc

SEQ ID NO: 53 - DNA, 44 bp, Artificial Sequence
  pstS3′ CAS
tttttaagcttgcatgcctgcagaggggagaaaagtcaggctaa

SEQ ID NO: 54 - DNA, 33 bp, Artificial Sequence
  Primer (n67)
tgcgaagacgtcctcgtgaagaaggtgttgctg

SEQ ID NO: 55 - DNA, 36 bp, Artificial Sequence
  Primer (n68)
tgcgaagggccccgttgtgtctcaaaatctctgatg

SEQ ID NO: 56 - DNA, 70 bp, Artificial Sequence
  Primer (ampH-attL-phi80)
atgcgcactccttacgtactggctctactggtttctttgcgaaaggtcatttttcctgaatatgctcaca

SEQ ID NO: 57 - DNA, 64 bp, Artificial Sequence
  Primer (ampH-attR-phi80)
ttaaggaatcgcctggaccatcatcggcgagccgttctgacgtttgttgacagctggtccaatg

SEQ ID NO: 58 - DNA, 68 bp, Artificial Sequence
  Primer (DampC-phL)
ctgatgaactgtcacctgaatgagtgctgatgaaaatatagaaaggtcatttttcctgaatatgctca

SEQ ID NO: 59 - DNA, 64 bp, Artificial Sequence
  Primer (DampC-phR)
attcgccagcataacgatgccgctgttgagctgaggaacacgtttgttgacagctggtccaatg

SEQ ID NO: 60 - DNA, 18 bp, Artificial Sequence
  Primer (ampH-t1)
gcgaagccctctccgttg

SEQ ID NO: 61 - DNA, 21 bp, Artificial Sequence
  Primer (ampH-t2)
agccagtcagcctcatcagcg

SEQ ID NO: 62 - DNA, 21 bp, Artificial Sequence
  Primer (ampC-t1)
gattcccacttcaccgagccg

SEQ ID NO: 63 - DNA, 21 bp, Artificial Sequence
  Primer (ampC-t2)
ggcaggtatggtgctctgacg

SEQ ID NO: 64 - DNA, 64 bp, Artificial Sequence
  Primer (crtE-attRphi80)
atgacggtctgcgcaaaaaaacacgttcatctcactcgcgcgtttgttgacagctggtccaatg

SEQ ID NO: 65 - DNA, 68 bp, Artificial Sequence
  Primer (crtZ-attLphi80)
atgttgtggatttggaatgccctgatcgttttcgttaccggaaaggtcatttttcctgaatatgctca

SEQ ID NO: 66 - DNA, 21 bp, Artificial Sequence
  Primer (crtZ-test)
ccgtgtggttctgaaagccga

SEQ ID NO: 67 - DNA, 21 bp, Artificial Sequence
  Primer (crtE-test)
cgttgccgtaaatgtatccgt

SEQ ID NO: 68 - DNA, 27 bp, Artificial Sequence
  Primer (phL-test)
ggatgtaaaccataacactctgcgaac

SEQ ID NO: 69 - DNA, 25 bp, Artificial Sequence
  Primer (phR-test)
gattggtggttgaattgtccgtaac

SEQ ID NO: 70 - DNA, 3471 bp, Artificial Sequence
  Sequence of the chemically synthesized DNA fragment retaining artificial KDyI operon with optimized codons
gcatgcaggaggtatgaatgtcagagttgcgtgccttcagtgccccagggaaagcgttactcgctggtggatatttagttttagatacaaaatatgaagcatttgtagtcggattatcggcacgtatgcacgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatcgtaacttgttcgttattgatattttctctgatgatgcctaccattctcaggaggatagcgttaccgaacatcgtggcaaccgccgtttgagttttcattcgcaccgtattgaagaagttcccaaaacagggctgggctcctcggcaggtttagtcacagttttaactacagctttggcctccttttttgtatcggacctggaaaataatgtagacaaatatcgtgaagttattcataatttagcacaagttgctcattgtcaagctcagggtaaaattggaagcgggtttgatgtagcggcggcagcatatggatctatccgttatcgccgtttcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcctcaaaatatatacagaactcgatcatgcaaattctcgttttatggatggactctctaaactcgatcgcttacacgagactcatgacgattacagcgatcagatatttgagtctcttgagcgtaatgactgtacctgtcaaaagtatcctgaaatcacagaagttcgtgatgcagttgccacaattcgtcgttcctttcgtaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttcgtgctcaaaccgctaatgacaaacgtttttctaaggttcaatggctggatgtaactcaggctgactggggtgttcgtaaagaaaaagatccggaaacttatcttgataaataactgcagaggaggtatgaatgaccgtttacacagcatccgttaccgcacccgtcaacatcgcaacccttaagtattgggggaaacgtgacacgaagttgaatctgcccaccaattcgtccatatcagtgactttatcgcaagatgacctccgtacgttgacctctgcggctactgcacctgagtttgaacgcgacactttgtggttaaatggagaaccacacagcatcgacaatgaacgtactcaaaattgtctgcgcgacctccgccaattacgtaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctcgtatagcacgtaaggggtctggttcagcttgtcgttcgttgtttggcggatacgtggcctgggaaatgggaaaagctgaagatggtcatgattccatggcagtacaaatcgcagacagctctgactggcctcagatgaaagcatgtgtccttgttgtcagcgatattaaaaaggatgtgagttccactcagggtatgcaattgaccgtggcaacctccgaactctttaaagaacgtattgaacatgtcgtaccaaagcgttttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgcccgtgtgattttaactcaagtcggttcaggcccacaagaaacaaacgaatctttgattgacgcaaagactggtctcccaaaggaataaggatccaggaggtatgaatgactgccgacaacaatagtatgccccatggtgcagtatctagttacgccaaattagtgcaaaaccaaacacctgaagacattttggaagagtttcctgaaattattccattacaacaacgtcctaatacccgctctagtgagacgtcaaatgacgaaagcggagaaacatgtttttctggtcatgatgaggagcaaattaagttaatgaatgaaaattgtattgttttggattgggacgataatgctattggtgccggcaccaagaaagtttgtcatttaatggaaaatattgaaaagggtttacttcatcgtgcattctccgtctttattttcaatgaacaaggtgaattacttttacaacaacgtgccactgaaaaaataactttccctgatctttggactaacacatgctgctctcatccactttgtattgatgacgaattaggtttgaagggtaagctcgacgataagattaagggcgctattactgcggcggtgcgtaaactcgatcatgaattaggtattccagaagatgaaactaagacacgtggtaagtttcactttttaaaccgtatccattacatggcaccaagcaatgaaccatggggtgaacatgaaattgattacatcctcttttataagatcaacgctaaagaaaacttgactgtcaacccaaacgtcaatgaagttcgtgacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgaccgtcaaattcatcgtatgctctaaggtacc

SEQ ID NO: 71 - DNA, 68 bp, Artificial Sequence
  Primer gcd-attL
ggtcaacattatggggaaaaactcctcatcctttagcgtgtgaagcctgcttttttatactaagttgg

SEQ ID NO: 72 - DNA, 68 bp, Artificial Sequence
  Primer gcd-attR
ttacttctggtcgggcagcgcataggcaatcacgtaatcgcgctcaagttagtataaaaaagctgaac

SEQ ID NO: 73 - DNA, 20 bp, Artificial Sequence
  Primer gcd-t1
tgacaacaatctatctgatt

SEQ ID NO: 74 - DNA, 20 bp, Artificial Sequence
  Primer gcd-t2
tgcgcctggttaagctggcg

SEQ ID NO: 75 - protein, 574 aa, Actinidia arguta
MASFNRFCVSSLLAPNNSPQISNAPRSTAVPSMPTTQKWSITEDLAFISNPSKQHNHQTGYRIFSDEFYLKHENKLKDVRRALREVEETPLEGLVMIDTLQRLGIDYHFQGEIGALLQKQQRISTCDYPEHDLFEVSTRFRLLRQEGHNVPADVFNNFRDKEGRFKSELSRDIRGLMSLYEASQLSIQGEDILDQAADFSSQLLSGWATNLDHHQARLVRNALTHPYHKSLATFMARNFNYDCKGQNGWVNNLQELAKMDLTMVQSMHQKEVLQVSQWWKGRGLANELKLVRNQPLKWYMWPMAALTDPRFSEERVELTKPISFIYIIDDIFDVYGTLEELTLFTDAVNRWELTAVEQLPDYMKICFKALYDITNEIAYKIYKKHGRNPIDSLRRTWASLCNAFLEEAKWFASGNLPKAEEYLKNGIISSGMHVVTVHMFFLLGGCFTEESVNLVDEHAGITSSIATILRLSDDLGSAKDEDQDGYDGSYLECYLKDHKGSSVENAREEVIRMISDAWKRLNEECLFPNPFSATFRKGSLNIARMVPLMYSYDDNHNLPILEEHMKTMLYDSSS

SEQ ID NO: 76 - DNA, 1725 bp, Actinidia arguta
atggccagcttcaacaggttttgtgtctcttctcttcttgctccaaacaacagcccacaaattagcaatgctccccgctccaccgctgtaccctctatgcctaccacccaaaaatggagcatcaccgaagacctagcattcatttctaatccctcgaaacaacacaaccatcaaaccggatatcgcattttctctgatgagttttacctaaagcacgaaaacaaattgaaggacgttaggagagcgttaagggaagtggaggaaaccccattagaaggtctggtcatgatcgacaccctccaacggctaggcattgactaccacttccagggggagattggagccctactacagaaacaacagagaatatctacttgtgattatcccgagcatgatctttttgaggtctctactcgctttcggctgttaaggcaagaaggtcacaatgtgcctgcagatgtgtttaacaacttcagagacaaggagggaaggttcaaatcagaactaagcagagacatcagggggttgatgagtttgtatgaagcttcacagttaagcatacaaggagaagacatacttgatcaagccgcagattttagttcccaactccttagcgggtgggcgacaaatctcgatcatcatcaagctaggcttgtgcgtaatgcactgacacatccctatcacaagagcctagcgacattcatggcaagaaacttcaattatgattgcaagggccaaaatggatgggtcaataacttgcaagaactagcaaaaatggacttaactatggttcagtccatgcatcaaaaagaagtccttcaagtttcccaatggtggaaaggcaggggtttggccaatgaattgaagcttgtgagaaatcagccacttaaatggtacatgtggccaatggcagccctcacagatccaagattctcagaggaaagagttgaactcacaaaaccaatctcttttatctatatcatagatgacatttttgatgtttatgggacattagaagaactcactctcttcacagatgctgtcaatagatgggaacttactgctgttgagcaactacccgactacatgaagatttgctttaaggctctttatgacatcacaaatgaaatcgcctacaagatctacaaaaagcatggacggaaccccatagattctctgcggagaacgtgggcaagtttgtgcaacgcgttcttagaagaagcaaaatggtttgcttctgggaacttgccaaaggcagaagagtacttgaagaatgggatcatcagttcagggatgcatgtggttacggttcacatgttctttctcttgggcggttgtttcaccgaagaaagtgtcaatcttgtggatgaacatgcgggaattacatcttctatagcaacaatccttcgtctttcggatgacttgggaagtgccaaggatgaggatcaagatggctacgatggatcctatttagaatgctatctgaaggaccacaagggctcttcggtagagaatgcaagagaagaagttattcgcatgatttcagatgcatggaagcgcctcaacgaggaatgcctatttccgaatccattttcagcaactttcaggaagggttctcttaatatcgcaaggatggttcctttgatgtacagctatgatgacaatcataacctcccaatccttgaggagcacatgaagacaatgctctatgatagttcttcttga

SEQ ID NO: 77 - DNA, 1650 bp, Artificial Sequence
  DNA having modified codons, which encodes linalool synthase gene derived from Actinidia arguta (opt_AaLINS)
atgtccaccgccgtgccctctatgcccactacccaaaaatggtctattaccgaagacttagcctttattagcaatcccagcaaacaacataatcaccaaaccggctaccggatttttagtgacgaattttacctgaaacatgaaaacaaattgaaagatgtgcggcgcgccttgcgtgaagttgaagaaacccccctggaaggcttggtgatgattgacactttacagcggctgggtattgattaccactttcaaggcgaaattggtgccttgttacagaaacaacagcgcattagtacctgtgactatcccgaacatgatttgtttgaagtgagcactcgctttcgtctgttgcgtcaagaaggtcacaatgtgcccgccgacgtttttaataactttcgcgataaagaagggcgttttaaatctgaactgtcccgggatattcgcggattgatgtccttatacgaagccagtcaactgagcattcagggggaagacattttggatcaagccgctgacttttccagtcagttactgtctggatgggccaccaatttagatcatcaccaagcccgtctggtgcggaacgctttgacccatccctaccacaaaagtctggccacttttatggctcgcaactttaactacgattgcaaagggcaaaacggatgggtgaataacctgcaggaattggccaaaatggatttaaccatggttcaaagtatgcatcagaaagaagtgctgcaagttagccagtggtggaaagggcggggattggccaatgaactgaaattggtgcgcaaccaacccttgaaatggtatatgtggcccatggccgctttaaccgatccccggttttctgaagaacgcgtggaattgactaaacccatttcctttatttacattattgatgacatttttgacgtttatggcaccttagaagaattaaccctgtttactgatgccgtgaatcggtgggaattaactgctgttgaacagctgcccgactacatgaaaatttgttttaaagccttgtacgatattaccaacgaaattgcttacaaaatttacaaaaaacatgggcgcaaccccattgatagtttacgtcggacttgggccagcttatgcaatgcttttctggaagaagccaaatggtttgctagtggcaatttgcccaaagccgaagaatacctgaaaaacgggattattagctctggaatgcatgtggttaccgtgcacatgtttttcttgttaggcggttgttttactgaagaatccgtgaatttggttgatgaacatgccggcattacctccagtattgctactattttgcgtttatctgatgacttaggttccgccaaagatgaagaccaagatggctatgacggtagctacttggaatgttacctgaaagatcataaaggtagctctgtggaaaatgcccgtgaagaagttattcggatgatttccgatgcttggaaacgcttgaatgaagaatgcttatttcccaaccccttttctgccacctttcgcaaagggtccttaaatattgctcgtatggtgcccctgatgtacagttacgatgacaaccataacctgcccattctggaagaacacatgaaaaccatgttgtatgattccagtagctaa

SEQ ID NO: 78 - protein, 590 aa, Coriandrum sativum
MAAITIFPLSYSIKFRRSSPCNPKDVTACKSVIKSVTGMTKVPVPVPEPIVRRSGNYKPCMWDNDFLQSLKTEYTGEAINARASEMKEEVRMIFNNVVEPLNQLELIDQLQRLGLDYHFRDEINHTLKNVHNGQKSETWEKDLHATALEFRLLRQHGHYISPEGFKRFTENGSFNKGIRADVRGLLSLYEASYFSIEGESLMEEAWSFTSNILKECLENTIDLDLQMQVRHALELPLQWRIPRFDAKWYINLYQRSGDMIPAVLEFAKLDFNIRQALNQEELKDLSRWWSRLDMGEKLPFARDRLVTSFFWSLGITGEPHHRYCREVLTKIIEFVGVYDDVYDVYGTLDELELFTNVVKRWDTNAMKELPDYMKLCFLSLINMVNETTYDILKDHNIDTLPHQRKWFNDLFERYIVEARWYNSGYQPTLEEYLKNGFVSIGGPIGVLYSYICTEDPIKKEDLEFIEDLPDIVRLTCEIFRLTDDYGTSSAELKRGDVPSSIYCYMSDTGVTEEVSRKHMMNLIRKKWAQINKLRFSKEYNNPLSWSFVDIMLNIIRAAHFLYNTGDDGFGVEDVAVEATLVSLLVEPIPL

SEQ ID NO: 79 - DNA, 1773 bp, Coriandrum sativum
atggcagcgataactatatttccactttcttattcgatcaaatttaggagatcctccccatgcaatcctaaagatgtgacagcctgcaagtctgtaattaaatccgtcactggaatgactaaggttcctgttccagtaccagagcctatcgtaaggcgatcagggaactacaaaccttgcatgtgggacaacgatttcttgcagtctttgaaaactgaatacaccggggaagcaatcaatgcacgagcttctgagatgaaggaagaggtgaggatgatatttaataatgtggtcgaaccattgaatcagcttgagctgattgatcagttgcagagacttgggttggattatcattttcgtgatgaaatcaaccatactttgaagaacgtacataatggtcagaagagtgagacttgggagaaggacttgcatgctactgctcttgaatttaggcttcttagacaacatggacattatatatcccctgagggcttcaagagatttacagagaatgggagcttcaataaaggtatccgtgcagatgtccggggactattaagtttatatgaagcctcgtacttttctattgaaggagagtccctgatggaggaggcttggtcctttacaagtaacatccttaaagagtgcctcgaaaatactattgatttggatctccagatgcaagtgagacatgctttggaacttccactacaatggaggatcccgagatttgatgcaaagtggtacataaatttgtatcaaagaagtggtgacatgatcccagcggttctggaatttgcaaagttggacttcaacattaggcaagcgttgaaccaagaagagcttaaagatttgtcgaggtggtggagtagattagacatgggagagaaacttccctttgccagagataggttggtaacatcatttttctggagtttggggattactggcgagcctcatcacagatattgcagagaggttttaaccaaaataatagagtttgttggtgtatacgatgatgtttatgatgtatatggtacacttgatgaacttgaactctttacaaatgtcgtgaagaggtgggatacaaatgcaatgaaagagctcccagactacatgaagttgtgcttcctgtcattgatcaacatggtcaatgaaacgacttacgacatcctcaaggaccataacatcgatactttaccacaccaaagaaaatggttcaatgatttattcgagcgttacatagtggaggcgaggtggtataacagtggataccagccaacactagaagaatacttgaaaaatggatttgtgtcaataggaggccccattggagtgctttactcttacatctgtactgaggatccaatcaagaaagaagatttagagtttatcgaggaccttcctgatatagtacgattgacatgtgaaatttttcggttaactgatgattatggaacatcttcggctgagttaaagagaggagatgttccatcttctatatattgctacatgtcggatactggtgttacggaagaagtttcccgtaagcacatgatgaacttgatcaggaagaagtgggcacaaattaacaaactcagattttcaaaggagtataataatcctttatcgtggtcttttgttgatattatgttgaatataatcagggcagcccattttttgtataatactggagacgatggctttggtgttgaagatgttgcagttgaagctacattagtttcgcttcttgtcgagcccattcctctctaa

SEQ ID NO: 80 - DNA, 1659 bp, Artificial Sequence
  DNA having modified codons, which encodes linalool synthase gene derived from Coriandrum sativum (opt_CsLINS)
atgactaaagtgcccgtgcccgtgcccgaacccattgtgcggcggagcggtaactataaaccctgtatgtgggataacgattttctgcaaagcttaaaaaccgaatatactggcgaagccattaatgcccgtgcttctgaaatgaaagaagaagtgcggatgatttttaacaacgtggttgaacccctgaaccaattggaactgattgatcaactgcagcgcctggggttggactaccattttcgtgatgaaattaaccataccttgaaaaacgtgcacaacggacaaaaatccgaaacctgggaaaaagatttacacgccactgctctggaatttcgtttgttacggcagcatggccactatattagccccgaaggttttaaacggtttaccgaaaatgggtcttttaacaaaggcattcgggccgatgtgcggggcctgttgtccctgtacgaagctagctacttttctattgaaggtgaaagtttgatggaagaagcctggtcctttactagtaacattttgaaagaatgtctggaaaacaccattgatttagacctgcaaatgcaggtgcgccatgccttggaattacccctgcaatggcgcattccccgttttgatgctaaatggtacattaacctgtaccagcgcagtggggacatgattcccgccgtgttggaatttgctaaactggattttaacattcgtcaagccttgaaccaggaagaattaaaagacctgagccgctggtggtctcgtctggatatgggcgaaaaattgccctttgctcgggatcgcttggtgacttcctttttctggagtttaggcattaccggtgaaccccatcaccggtactgtcgcgaagttctgaccaaaattattgaatttgtgggggtttacgatgacgtgtatgacgtttacggaaccttggatgaattggaactgtttactaacgtggttaaacgttgggacaccaacgccatgaaagaattacccgattatatgaaactgtgctttctgtccttgattaatatggtgaacgaaaccacttacgatattctgaaagaccataacattgataccttgccccaccaacgcaaatggtttaacgatctgtttgaacggtacattgtggaagcccgctggtataatagtggttaccagcccaccctggaagaatacttgaaaaatgggtttgtgtccattggcggtcccattggagttttgtacagttacatttgtactgaagaccccatcaaaaaagaagatttggaatttattgaagatttacccgacattgtgcgtctgacctgcgaaatttttcggctgaccgatgactatggcacttccagtgccgaattgaaacggggtgacgttcccagctctatttattgctacatgagcgataccggtgtgactgaagaagtttctcggaaacatatgatgaacctgattcgcaaaaaatgggcccaaattaacaaactgcggtttagcaaagaatataataaccccttgtcctggagttttgtggatattatgctgaacattattcgcgccgctcattttctgtacaacactggggatgacgggtttggagttgaagatgtggccgttgaagctaccttagtgagtttactggttgaacccattcccttataa

SEQ ID NO: 81 - DNA, 900 bp, Escherichia coli
atggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 82 - DNA, 900 bp, Artificial Sequence
  DNA having modified codons, which encodes farnesyl diphosphate synthase gene derived from Escherichia coli (ispA gene)
atggattttccccagcagctggaagcctgcgtgaaacaggccaaccaggccctgagccgctttatcgcccccctgccctttcagaacacccccgtggtggaaaccatgcagtacggcgccctgctgggcggcaaacgcctgcgcccctttctggtgtacgccaccggccacatgtttggcgtgagcaccaacaccctggatgcccccgccgccgccgtggaatgcatccacgcctactttctgatccacgatgatctgcccgccatggatgatgatgatctgcgccgcggcctgcccacctgccacgtgaaatttggcgaagccaacgccatcctggccggcgatgccctgcagaccctggcctttagcatcctgagcgatgccgatatgcccgaagtgagcgatcgcgatcgcatcagcatgatcagcgaactggccagcgccagcggcatcgccggcatgtgcggcggccaggccctggatctggatgccgaaggcaaacacgtgcccctggatgccctggaacgcatccaccgccacaaaaccggcgccctgatccgcgccgccgtgcgcctgggcgccctgagcgccggcgataaaggccgccgcgccctgcccgtgctggataaatacgccgaaagcatcggcctggcctttcaggtgcaggatgatatcctggatgtggtgggcgataccgccaccctgggcaaacgccagggcgccgatcagcagctgggcaaaagcacctaccccgccctgctgggcctggaacaggcccgcaaaaaagcccgcgatctgatcgatgatgcccgccagagcctgaaacagctggccgaacagagcctggataccagcgccctggaagccctggccgattacatcatccagcgcaacaaatag

SEQ ID NO: 83 - DNA, 37 bp, Artificial Sequence
  Primer
acgttgttgccattgccctgttgacaattaatcatcg

SEQ ID NO: 84 - DNA, 37 bp, Artificial Sequence
  Primer
atgacttggttgagtttagctactggaatcatacaac

SEQ ID NO: 85 - DNA, 29 bp, Artificial Sequence
  Primer
tgtgaaattagctactggaatcatacaac

SEQ ID NO: 86 - DNA, 45 bp, Artificial Sequence
  Primer
gtagctaatttcacacaggagactgccatggattttccccagcag

SEQ ID NO: 87 - DNA, 37 bp, Artificial Sequence
  Primer
atgacttggttgagtctatttgttgcgctggatgatg

SEQ ID NO: 88 - DNA, 28 bp, Artificial Sequence
  Primer
tgtgaaattataagggaatgggttcaac

SEQ ID NO: 89 - DNA, 45 bp, Artificial Sequence
  Primer
ccttataatttcacacaggagactgccatggattttccccagcag

SEQ ID NO: 90 - protein, 634 aa, Picea sitchensis
MSPVSAIPLAYKLCLPRSLISSSRELNPLHITIPNLGMCRRGKSMAPASMSMILTAAVSDDDRVQRRRGNYHSNLWDDDFIQSLSTPYGEPSYRESAERLKGEIKKMFRSMSKEDEELITPLNDLIQRLWMVDSVERLGIDRHFKNEIKSALDYVYSYWNEKGIGCGRDSVVADLNSTALGFRTLRLHGYNVSSEVLKVFEDQNGQFACSPSKTEGEIRSALNLYRASLIAFPGEKVMEDAEIFSSRYLKEAVQKIPDCSLSQEIAYALEYGWHTNMPRLEARNYMDVFGHPSSPWLKKNKTQYMDGEKLLELAKLEFNIFHSLQQEELQYISRWWKDSGLPKLAFSRHRHVEYYTLGSCIATDPKHRAFRLGFVKTCHLNTVLDDIYDTFGTMDEIELFTEAVRRWDPSETESLPDYMKGVYMVLYEALTEMAQEAEKTQGRDTLNYARKAWEIYLDSYIQEAKWIASGYLPTFQEYFENGKISSAYRAAALTPILTLDVPLPEYILKGIDFPSRFNDLASSFLRLRGDTRCYKADRARGEEASCISCYMKDNPGSTEEDALNHINSMINEIIKELNWELLRPDSNIPMPARKHAFDITRALHHLYKYRDGFSVATKETKSLVSRMVLEPVTL

SEQ ID NO: 91 - DNA, 1905 bp, Picea sitchensis
atgtctcctgtttctgccataccgttggcttacaaattgtgcctgcccagatcgttgatcagttctagtcgtgagcttaatcctctccatataacaatcccaaatcttggaatgtgcaggcgagggaaatcaatggcaccagcttccatgagcatgattttgaccgccgccgtctctgatgatgaccgtgtacaaagacgcagaggcaattatcactcgaacctctgggacgatgatttcatacagtctctttcaacgccttatggggaaccttcttatcgggaaagtgctgagagacttaaaggggaaataaagaagatgttcagatcaatgtcaaaggaggatgaagaattaattactcccctcaatgatctcattcaacgactttggatggtcgatagcgtcgaacgtttggggatcgatagacatttcaaaaatgagataaaatcagcgctggattatgtttacagttattggaatgaaaaaggcattggatgtgggagagatagtgttgttgctgatctcaactccactgccttggggtttcgaactcttcgcctacacggatacaatgtctcctcagaggttttgaaagtttttgaagaccaaaacggacagtttgcatgctctcccagtaaaacagaaggggagatcagaagcgctcttaacttatatcgggcttccctcattgcctttcctggggagaaagttatggaagacgctgaaatcttctcttcaagatatttgaaagaagccgtgcaaaagattccggactgcagtctttcacaagagatagcctatgctttggaatatggttggcacacaaatatgccaagattggaagcaaggaattacatggacgtatttggacatcctagtagcccatggctcaagaagaataagacgcaatatatggacggcgagaaacttttagaactagcaaaattggagttcaatatctttcactccttgcaacaggaggagttacaatatatctccagatggtggaaagattcgggtttgcctaaactggccttcagtcggcatcgtcacgtggaatactacactttggggtcttgcattgcgactgaccccaaacatcgtgcattcagactgggctttgtcaaaacgtgtcatcttaacacggttctggacgatatctacgacacattcggaacgatggacgaaatcgaactcttcacagaagcagtcaggagatgggatccgtcggagacagagagccttccagactatatgaaaggagtgtacatggtactctacgaagccctaactgaaatggctcaagaggcggagaaaacacaaggccgagacacgctcaactatgctcgaaaggcttgggagatttatcttgattcgtatattcaagaagcaaagtggatcgccagtggttatctgccaacatttcaggaatactttgagaacgggaaaattagctctgcttatcgcgcagcggcattgacacccatcctcacattggacgtaccgcttcctgaatacatcttgaagggaattgattttccatcgagattcaatgatttggcatcttccttccttcgactaagaggtgacacacgctgctacaaggcggatagggcccgtggagaagaagcttcgtgcatatcttgttatatgaaagacaatcctggatcaacggaggaagatgctctcaatcatatcaactccatgatcaatgaaataatcaaagaattaaattgggaattactaagacctgatagcaatattccaatgcctgcgaggaaacatgcttttgacataactagagctctccaccacctctataaataccgagatgggttcagcgttgccactaaggaaacgaaaagtctggtcagcagaatggtccttgaacctgtgactttgtaa

SEQ ID NO: 92 - DNA, 1716 bp, Artificial Sequence
  DNA having modified codons, which encodes limonene synthase gene derived from Picea sitchensis (opt_PsLMS)
atgcagcgccgtcgcggcaattaccacagcaacctgtgggacgatgactttatccagagtctgagcaccccgtatggtgaacccagttaccgtgaaagcgcggagcgcctgaaaggcgagattaaaaagatgtttcgcagtatgagcaaggaagatgaagagctgatcacgccgctgaatgacctgattcagcgcctgtggatggtcgatagcgttgagcgtctgggcatcgaccgccattttaaaaatgaaattaagagtgccctggattacgtctatagctactggaacgaaaaaggcatcggttgtggccgcgatagtgttgtggcggacctgaatagcaccgccctgggttttcgtacgctgcgcctgcacggctacaatgttagtagcgaggtgctgaaagtctttgaagatcagaacggccagtttgcgtgcagtccgagcaagaccgaaggcgagatccgtagtgccctgaacctgtatcgcgcgagcctgattgcctttcccggtgaaaaagttatggaagacgcggagatttttagtagccgctacctgaaagaagccgtgcagaagatccccgattgtagtctgagccaggagattgcgtatgccctggaatacggctggcataccaatatgcctcgtctggaagcccgcaactatatggacgtttttggtcaccccagtagcccttggctgaaaaagaataagacgcagtacatggatggcgaaaaactgctggagctggcgaagctggaatttaacatctttcatagcctgcagcaggaagagctgcagtacattagtcgttggtggaaagacagcggcctgcctaagctggcctttagccgtcatcgccacgtggaatactataccctgggtagctgtatcgcgacggatccgaaacatcgtgcctttcgcctgggctttgtgaagacctgccacctgaatacggtcctggatgacatctatgatacctttggcacgatggacgaaattgagctgtttaccgaagcggtccgtcgctgggatccgagtgaaacggagagcctgcccgactacatgaaaggtgtttatatggtgctgtacgaagccctgaccgagatggcgcaggaagccgagaaaacccagggccgtgacacgctgaattatgcgcgcaaggcctgggagatttatctggatagttacatccaggaagcgaaatggattgccagcggttatctgccgacgtttcaggaatactttgagaacggcaagatcagtagcgcctatcgtgcggccgccctgacccctattctgaccctggatgtgccgctgcccgaatacattctgaaaggcatcgattttccgagccgttttaatgacctggccagtagctttctgcgtctgcgcggtgatacccgctgctataaggccgaccgtgcccgcggcgaagaggcgagttgtattagctgctacatgaaggataatcccggcagtacggaagaggacgccctgaaccatatcaacagcatgatcaacgaaatcatcaaggagctgaactgggaactgctgcgccctgatagcaacatccctatgccggcgcgtaaacacgcctttgacattacccgcgcgctgcatcacctgtataagtaccgtgatggctttagcgttgccaccaaagaaacgaagagtctggtcagccgcatggttctggaacccgtgacgctgtaa

SEQ ID NO: 93 - protein, 637 aa, Abies grandis
MALLSIVSLQVPKSCGLKSLISSSNVQKALCISTAVPTLRMRRRQKALVINMKLTTVSHRDDNGGGVLQRRIADHHPNLWEDDFIQSLSSPYGGSSYSERAETVVEEVKEMFNSIPNNRELFGSQNDLLTRLWMVDSIERLGIDRHFQNEIRVALDYVYSYWKEKEGIGCGRDSTFPDLNSTALALRTLRLHGYNVSSDVLEYFKDEKGHFACPAILTEGQITRSVLNLYRASLVAFPGEKVMEEAEIFSASYLKKVLQKIPVSNLSGEIEYVLEYGWHTNLPRLEARNYIEVYEQSGYESLNEMPYMNMKKLLQLAKLEFNIFHSLQLRELQSISRWWKESGSSQLTFTRHRHVEYYTMASCISMLPKHSAFRMEFVKVCHLVTVLDDIYDTFGTMNELQLFTDAIKRWDLSTTRWLPEYMKGVYMDLYQCINEMVEEAEKTQGRDMLNYIQNAWEALFDTFMQEAKWISSSYLPTFEEYLKNAKVSSGSRIATLQPILTLDVPLPDYILQEIDYPSRFNELASSILRLRGDTRCYKADRARGEEASAISCYMKDHPGSIEEDALNHINAMISDAIRELNWELLRPDSKSPISSKKHAFDITRAFHHVYKYRDGYTVSNNETKNLVMKTVLEPLAL

SEQ ID NO: 94 - DNA, 1914 bp, Abies grandis
atggctctcctttctatcgtatctttgcaggttcccaaatcctgcgggctgaaatcgttgatcagttccagcaatgtgcagaaggctctctgtatctctacagcagtcccaacactcagaatgcgtaggcgacagaaagctctggtcatcaacatgaaattgaccactgtatcccatcgtgatgataatggtggtggtgtactgcaaagacgcatagccgatcatcatcccaacctgtgggaagatgatttcatacaatcattgtcctcaccttatgggggatcttcgtacagtgaacgtgctgagacagtcgttgaggaagtaaaagagatgttcaattcaataccaaataatagagaattatttggttcccaaaatgatctccttacacgcctttggatggtggatagcattgaacgtctggggatagatagacatttccaaaatgagataagagtagccctcgattatgtttacagttattggaaggaaaaggaaggcattgggtgtggcagagattctacttttcctgatctcaactcgactgccttggcgcttcgaactcttcgactgcacggatacaatgtgtcttcagatgtgctggaatacttcaaagatgaaaaggggcattttgcctgccctgcaatcctaaccgagggacagatcactagaagtgttctaaatttatatcgggcttccctggtcgcctttcccggggagaaagttatggaagaggctgaaatcttctcggcatcttatttgaaaaaagtcttacaaaagattccggtctccaatctttcaggagagatagaatatgttttggaatatggttggcacacgaatttgccgagattggaagcaagaaattatatcgaggtctacgagcagagcggctatgaaagcttaaacgagatgccatatatgaacatgaagaagcttttacaacttgcaaaattggagttcaatatctttcactctttgcaactaagagagttacaatctatctccagatggtggaaagaatcaggttcgtctcaactgacttttacacggcatcgtcacgtggaatactacactatggcatcttgcatttctatgttgccaaaacattcagctttcagaatggagtttgtcaaagtgtgtcatcttgtaacagttctcgatgatatatatgacacttttggaacaatgaacgaactccaactttttacggatgcaattaagagatgggatttgtcaacgacaaggtggcttccagaatatatgaaaggagtgtacatggacttgtatcaatgcattaatgaaatggtggaagaggctgagaagactcaaggccgagatatgctcaactatattcaaaatgcttgggaagccctatttgatacctttatgcaagaagcaaagtggatctccagcagttatctcccaacgtttgaggagtacttgaagaatgcaaaagttagttctggttctcgcatagccacattacaacccattctcactttggatgtaccacttcctgattacatactgcaagaaattgattatccatccagattcaatgagttagcttcgtccatccttcgactacgaggtgacacgcgctgctacaaggcggatagggcccgtggagaagaagcttcagctatatcgtgttatatgaaagaccatcctggatcaatagaggaagatgctctcaatcatatcaacgccatgatcagtgatgcaatcagagaattaaattgggagcttctcagaccggatagcaaaagtcccatctcttccaagaaacatgcttttgacatcaccagagctttccatcatgtctacaaatatcgagatggttacactgtttccaacaacgaaacaaagaatttggtgatgaaaaccgttcttgaacctctcgctttgtaa

SEQ ID NO: 95 - DNA, 1713 bp, Artificial Sequence
  DNA having modified codons, which encodes limonene synthase gene derived from Abies grandis (opt_AgLMS)
atgcagcgtcgcatcgcggatcatcaccccaatctgtgggaagatgactttattcagagcctgagtagcccttatggtggcagtagctacagtgaacgcgccgagaccgttgtggaagaggttaaggaaatgtttaacagcatccccaacaaccgtgagctgtttggcagtcagaacgatctgctgacgcgcctgtggatggtggatagcatcgaacgtctgggtattgaccgccactttcagaatgaaatccgcgttgcgctggactacgtgtatagctactggaaagaaaaagaaggcattggctgtggtcgcgatagcacctttcctgacctgaatagtacggccctggccctgcgtaccctgcgtctgcatggctataacgtcagtagcgatgttctggaatactttaaagacgagaagggccactttgcctgccccgcgatcctgaccgagggtcagattacgcgtagcgttctgaatctgtatcgcgccagtctggtggcgtttcctggcgaaaaagtcatggaagaggccgagatctttagcgcgagttacctgaaaaaggtcctgcagaagatccctgttagcaacctgagtggcgaaattgagtatgtgctggaatacggttggcataccaatctgccgcgtctggaagcccgcaactatattgaagtctacgagcagagcggctatgaaagtctgaatgagatgccgtacatgaacatgaaaaagctgctgcagctggcgaaactggaatttaatatctttcacagcctgcagctgcgtgaactgcagagcattagtcgttggtggaaggagagcggtagtagccagctgacctttacgcgtcatcgccacgtggaatactatacgatggccagctgtatcagtatgctgcctaaacatagcgcgtttcgcatggaatttgtcaaggtttgccacctggtgaccgtcctggatgacatctacgatacctttggcacgatgaatgaactgcagctgtttacggatgccattaaacgttgggacctgagcaccacccgttggctgcccgaatacatgaagggcgtttatatggacctgtaccagtgtattaacgaaatggtggaagaggccgagaaaacccagggtcgcgatatgctgaattacatccagaacgcctgggaagcgctgtttgacacctttatgcaggaagccaagtggattagtagcagttatctgcccacgtttgaagagtacctgaaaaatgccaaagtcagcagcggtagccgtattgccaccctgcagccgattctgacgctggatgttccgctgcccgactatattctgcaggaaatcgattaccccagccgttttaacgagctggccagcagtattctgcgtctgcgcggcgatacgcgctgttataaagcggaccgtgcccgcggtgaagaggccagcgcgatcagttgctacatgaaggatcatccgggcagcattgaagaggacgcgctgaatcacattaacgccatgatcagtgatgcgattcgtgaactgaattgggagctgctgcgccctgacagcaaaagtccgatcagcagtaaaaagcatgcctttgatattacccgtgcgtttcatcacgtctataagtaccgcgacggctacaccgttagcaataacgaaacgaaaaacctggtgatgaagacggtcctggaacccctggcgctgtaa

SEQ ID NO: 96 - protein, 599 aa, Mentha spicata
MALKVLSVATQMAIPSNLTTCLQPSHFKSSPKLLSSTNSSSRSRLRVYCSSSQLTTERRSGNYNPSRWDVNFIQSLLSDYKEDKHVIRASELVTLVKMELEKETDQIRQLELIDDLQRMGLSDHFQNEFKEILSSIYLDHHYYKNPFPKEERDLYSTSLAFRLLREHGFQVAQEVFDSFKNEEGEFKESLSDDTRGLLQLYEASFLLTEGETTLESAREFATKFLEEKVNEGGVDGDLLTRIAYSLDIPLHWRIKRPNAPVWIEWYRKRPDMNPVVLELAILDLNIVQAQFQEELKESFRWWRNTGFVEKLPFARDRLVECYFWNTGIIEPRQHASARIMMGKVNALITVIDDIYDVYGTLEELEQFTDLIRRWDINSIDQLPDYMQLCFLALNNFVDDTSYDVMKEKGVNVIPYLRQSWVDLADKYMVEARWFYGGHKPSLEEYLENSWQSISGPCMLTHIFFRVTDSFTKETVDSLYKYHDLVRWSSFVLRLADDLGTSVEEVSRGDVPKSLQCYMSDYNASEAEARKHVKWLIAEVWKKMNAERVSKDSPFGKDFIGCAVDLGRMAQLMYHNGDGHGTQHPIIHQQMTRTLFEPFA

SEQ ID NO: 97 - DNA, 1800 bp, Mentha spicata
atggctctcaaagtgttaagtgttgcaactcaaatggcgattcctagcaacctaacgacatgtcttcaaccctcacacttcaaatcttctccaaaactgttatctagcactaacagtagtagtcggtctcgcctccgtgtgtattgctcctcctcgcaactcactactgaaagacgatccggaaactacaacccttctcgttgggatgtcaacttcatccaatcgcttctcagtgactataaggaggacaaacacgtgattagggcttctgagctggtcactttggtgaagatggaactggagaaagaaacggatcaaattcgacaacttgagttgatcgatgacttgcagaggatggggctgtccgatcatttccaaaatgagttcaaagaaatcttgtcctctatatatctcgaccatcactattacaagaacccttttccaaaagaagaaagggatctctactccacatctcttgcatttaggctcctcagagaacatggttttcaagtcgcacaagaggtattcgatagtttcaagaacgaggagggtgagttcaaagaaagccttagcgacgacaccagaggattgttgcaactgtatgaagcttcctttctgttgacggaaggcgaaaccacgctcgagtcagcgagggaattcgccaccaaatttttggaggaaaaagtgaacgagggtggtgttgatggcgaccttttaacaagaatcgcatattctttggacatccctcttcattggaggattaaaaggccaaatgcacctgtgtggatcgaatggtataggaagaggcccgacatgaatccagtagtgttggagcttgccatactcgacttaaatattgttcaagcacaatttcaagaagagctcaaagaatccttcaggtggtggagaaatactgggtttgttgagaagctgcccttcgcaagggatagactggtggaatgctacttttggaatactgggatcatcgagccacgtcagcatgcaagtgcaaggataatgatgggcaaagtcaacgctctgattacggtgatcgatgatatttatgatgtctatggcaccttagaagaactcgaacaattcactgacctcattcgaagatgggatataaactcaatcgaccaacttcccgattacatgcaactgtgctttcttgcactcaacaacttcgtcgatgatacatcgtacgatgttatgaaggagaaaggcgtcaacgttataccctacctgcggcaatcgtgggttgatttggcggataagtatatggtagaggcacggtggttctacggcgggcacaaaccaagtttggaagagtatttggagaactcatggcagtcgataagtgggccctgtatgttaacgcacatattcttccgagtaacagattcgttcacaaaggagaccgtcgacagtttgtacaaataccacgatttagttcgttggtcatccttcgttctgcggcttgctgatgatttgggaacctcggtggaagaggtgagcagaggggatgtgccgaaatcacttcagtgctacatgagtgactacaatgcatcggaggcggaggcgcggaagcacgtgaaatggctgatagcggaggtgtggaagaagatgaatgcggagagggtgtcgaaggattctccattcggcaaagattttataggatgtgcagttgatttaggaaggatggcgcagttgatgtaccataatggagatgggcacggcacacaacaccctattatacatcaacaaatgaccagaaccttattcgagccctttgcatga

SEQ ID NO: 98 - DNA, 1635 bp, Artificial Sequence
  DNA having modified codons, which encodes limonene synthase gene derived from Mentha spicata (opt_MsLMS)
atggaacgccgtagcggcaattataaccccagtcgttgggatgttaattttattcagagcctgctgagtgattacaaagaggacaagcacgtgatccgcgcgagcgaactggttaccctggtgaaaatggaactggagaaggaaacggatcagatccgtcagctggaactgattgatgacctgcagcgcatgggcctgagcgaccattttcagaatgagtttaaggaaatcctgagcagtatctacctggatcatcactactacaagaacccgtttcccaaggaagagcgcgacctgtatagcacgagtctggcctttcgtctgctgcgcgagcatggctttcaggtggcgcaggaagtctttgatagctttaaaaacgaagagggtgaatttaaggaaagcctgagtgatgacacccgcggcctgctgcagctgtacgaagccagttttctgctgacggagggtgaaaccacgctggagagcgcccgcgaatttgcgaccaaatttctggaagagaaggttaatgaaggcggtgtggatggtgacctgctgacccgtatcgcctacagcctggatattccgctgcactggcgtatcaaacgccctaacgcgccggtctggattgagtggtatcgtaagcgccccgatatgaatcctgttgtgctggaactggccatcctggacctgaacattgtgcaggcgcagtttcaggaagagctgaaagagagctttcgttggtggcgcaatacgggctttgtcgaaaagctgccgtttgcccgtgatcgcctggttgagtgttacttttggaataccggtattatcgaaccccgtcagcatgccagcgcgcgcattatgatgggcaaagtcaacgcgctgattacggttatcgatgacatttacgacgtttatggcaccctggaagagctggaacagtttacggatctgatccgccgttgggacatcaatagcattgatcagctgcctgactacatgcagctgtgctttctggcgctgaataactttgttgatgacaccagttacgatgtgatgaaagaaaagggtgtcaacgttatcccgtatctgcgtcagagctgggtggatctggccgacaaatacatggtcgaagcgcgctggttttatggcggtcacaagcccagcctggaagagtatctggaaaatagttggcagagcattagtggcccttgtatgctgacccacattttctttcgcgttacggatagctttaccaaagaaacggtggatagtctgtacaagtatcatgacctggtgcgttggagcagttttgtcctgcgcctggccgatgacctgggtacgagcgtcgaagaggtttcacgcggcgatgtgcccaaaagcctgcagtgttacatgagcgactataatgcgagtgaggccgaagcgcgtaaacacgtcaagtggctgattgccgaggtttggaaaaagatgaacgcggaacgcgtgagcaaagatagtccttttggcaaggactttattggttgtgcggttgatctgggtcgtatggcgcagctgatgtaccataatggcgacggtcatggcacccagcaccctattatccatcagcagatgacccgcacgctgtttgaaccgtttgcctaa

SEQ ID NO: 99 - protein, 608 aa, Citrus unshiu
MSSCINPSTLATSVNGFKCLPLATNRAAIRIMAKNKPVQCLVSTKYDNLTVDRRSANYQPSIWDHDFLQSLNSNYTDETYKRRAEELKGKVKTAIKDVTEPLDQLELIDNLQRLGLAYHFEPEIRNILRNIHNHNKDYNWRKENLYATSLEFRLLRQHGYPVSQEVFSGFKDDKVGFICDDFKGILSLHEASYYSLEGESIMEEAWQFTSKHLKEMMITSNSKEEDVFVAEQAKRALELPLHWKKVPMLEARWFIHVYEKREDKNHLLLELAKLEFNTLQAIYQEELKDISGWWKDTGLGEKLSFARNRLVASFLWSMGIAFEPQFAYCRRVLTISIALITVIDDIYDVYGTLDELEIFTDAVARWDINYALKHLPGYMKMCFLALYNFVNEFAYYVLKQQDFDMLLSIKHAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVAREHIKDMMRQMWKKVNAYTADKDSPLTRTTAEFLLNLVRMSHFMYLHGDGHGVQNQETIDVGFTLLFQPIPLEDKDMAFTASPGTKG

SEQ ID NO: 100 - DNA, 1827 bp, Citrus unshiu
atgtcttcttgcattaatccctcaaccttggctacctctgtaaatggtttcaaatgtcttcctcttgcaacaaatagagcagccatcagaatcatggcaaaaaataagccagtccaatgccttgtcagcaccaaatatgataatttgacagttgataggagatcagcaaactaccaaccttcaatttgggaccatgattttttgcagtcactgaatagcaactatacggatgaaacatacaaaagacgagcagaagagctgaagggaaaagtgaagacagcgattaaggatgtaaccgagcctctggatcagttggagctgattgataatttgcaaagacttggattggcttatcattttgagcctgagattcggaacatattgcgtaatatccacaaccataataaagattataattggagaaaagaaaatctgtatgcaacctcccttgaattcagacttcttagacaacatggctatcctgtttctcaagaggttttcagtggttttaaagacgacaaggtaggcttcatttgtgatgatttcaagggaatactgagcttgcatgaagcctcgtattacagcttagaaggagaaagcatcatggaggaggcctggcaattcaccagtaagcatcttaaagaaatgatgatcaccagcaacagcaaggaagaggatgtatttgtagcagaacaagcgaagcgggcgctggagctccctctgcattggaaaaaagtgcctatgttagaggcaaggtggttcatacacgtttatgagaaaagagaggacaagaaccaccttttacttgagctcgctaagttggagtttaacactttgcaggcaatttaccaggaagaacttaaagacatttcagggtggtggaaggatacaggtcttggagagaaattgagctttgcgaggaacaggttggtagcgtccttcttatggagcatggggatcgcgtttgagcctcaattcgcctactgcaggagagtgctcacaatctcgatagccctaattacagtgattgatgacatttatgatgtctatggaacattggatgaacttgagatattcactgatgctgttgcgaggtgggacatcaattatgctttgaagcaccttccgggctatatgaaaatgtgttttcttgccctttacaactttgttaatgaatttgcttattacgttctcaaacaacaggattttgatatgcttctgagcataaaacatgcatggcttggcttaatacaagcctacttggtggaggcgaaatggtaccatagcaagtacacaccgaaactggaagaatacttggaaaatggattggtatcaataacgggccctttaattataacgatttcatatctttctggtacaaatccaatcattaagaaggaactggaatttctagaaagtaatccagatatagttcactggtcatccaagattttccgtctgcaagatgatttgggaacttcatcggacgagatacagagaggggatgttccgaaatcaatccagtgttacatgcatgaaactggtgcctcggaggaagttgctcgtgaacacatcaaggatatgatgagacagatgtggaagaaggtgaatgcatacacagccgataaagactctcccttgactcgaacaactgctgagttcctcttgaatcttgtgcgaatgtcccattttatgtatctacatggagatgggcatggtgttcaaaaccaagagactatcgatgtcggctttacattgctttttcagcccattcccttggaggacaaagacatggctttcacagcatctcctggcaccaaaggctga

SEQ ID NO: 101 - DNA, 1677 bp, Artificial Sequence
  DNA having modified codons, which encodes limonene synthase gene derived from Citrus unshiu (opt_CuLMS)
atggaccgccgtagcgccaactatcagccgagtatttgggatcatgactttctgcagagcctgaatagtaactacaccgatgaaacgtataaacgccgtgcggaagagctgaaaggcaaggttaaaaccgccatcaaggatgtgacggaaccgctggaccagctggagctgattgataatctgcagcgcctgggcctggcgtatcattttgaacccgagattcgcaatatcctgcgtaacatccataatcacaacaaggattacaattggcgcaaagaaaacctgtatgccacgagcctggagtttcgtctgctgcgtcagcatggctaccccgtgagccaggaagtctttagtggttttaaggatgacaaagtcggctttatctgcgatgactttaaaggtattctgagcctgcatgaagcgagctactatagtctggaaggcgagagcatcatggaagaggcctggcagtttaccagtaagcacctgaaggaaatgatgatcacgagcaacagtaaagaagaggacgtttttgttgcggaacaggccaagcgtgccctggagctgcctctgcattggaaaaaggtcccgatgctggaagcccgctggtttatccatgtttatgaaaagcgtgaggataaaaatcacctgctgctggaactggcgaaactggagtttaacaccctgcaggccatctaccaggaagagctgaaggacattagcggttggtggaaagatacgggcctgggtgaaaagctgagttttgcgcgcaatcgtctggttgccagctttctgtggagtatgggcattgcgtttgaaccgcagtttgcctactgtcgccgtgtgctgaccattagcatcgcgctgattacggtcatcgatgacatttacgacgtttatggtaccctggatgaactggagatctttacggacgccgtggcgcgctgggatattaactacgccctgaaacacctgcccggctatatgaagatgtgctttctggcgctgtacaattttgtgaacgaatttgcctactatgtcctgaaacagcaggattttgacatgctgctgagcatcaagcatgcgtggctgggcctgattcaggcgtacctggtcgaagccaaatggtaccacagcaagtatacccctaaactggaagagtatctggagaatggtctggttagcatcaccggccccctgattatcacgattagctacctgagtggcacgaatcctattatcaaaaaggaactggagtttctggaaagcaacccggacatcgtgcattggagcagtaaaatttttcgcctgcaggatgacctgggtaccagcagtgacgaaatccagcgcggcgatgtccccaagagcattcagtgttacatgcatgaaaccggtgccagtgaagaggtggcccgtgagcacattaaagatatgatgcgtcagatgtggaaaaaggtcaatgcgtataccgccgataaagacagccctctgacccgtaccaccgccgaatttctgctgaatctggttcgtatgagtcactttatgtacctgcatggcgacggtcacggcgttcagaaccaggaaaccatcgatgtgggctttacgctgctgtttcagccgattcccctggaggataaagacatggcgtttaccgccagcccgggtacgaagggctaa

SEQ ID NO: 102 - protein, 606 aa, Citrus limon
MSSCINPSTLVTSVNAFKCLPLATNKAAIRIMAKYKPVQCLISAKYDNLTVDRRSANYQPSIWDHDFLQSLNSNYTDEAYKRRAEELRGKVKIAIKDVIEPLDQLELIDNLQRLGLAHRFETEIRNILNNIYNNNKDYNWRKENLYATSLEFRLLRQHGYPVSQEVFNGFKDDQGGFICDDFKGILSLHEASYYSLEGESIMEEAWQFTSKHLKEVMISKNMEEDVFVAEQAKRALELPLHWKVPMLEARWFIHIYERREDKNHLLLELAKMEFNTLQAIYQEELKEISGWWKDTGLGEKLSFARNRLVASFLWSMGIAFEPQFAYCRRVLTISIALITVIDDIYDVYGTLDELEIFTDAVERWDINYALKHLPGYMKMCFLALYNFVNEFAYYVLKQQDFDLLLSIKNAWLGLIQAYLVEAKWYHSKYTPKLEEYLENGLVSITGPLIITISYLSGTNPIIKKELEFLESNPDIVHWSSKIFRLQDDLGTSSDEIQRGDVPKSIQCYMHETGASEEVARQHIKDMMRQMWKKVNAYTADKDSPLTGTTTEFLLNLVRMSHFMYLHGDGHGVQNQETIDVGFTLLFQPIPLEDKHMAFTASPGTKG

SEQ ID NO: 103 - DNA, 1821 bp, Citrus limon
atgtcttcttgcattaatccctcaaccttggttacctctgtaaatgctttcaaatgtcttcctcttgcaacaaataaagcagccatcagaatcatggccaaatataagccagtccaatgccttatcagcgccaaatatgataatttgacagttgataggagatcagcaaactaccaaccttcaatttgggaccacgattttttgcagtcattgaatagcaactatacggatgaagcatacaaaagacgagcagaagagctgaggggaaaagtgaagatagcgattaaggatgtaatcgagcctctggatcagttggagctgattgataacttgcaaagacttggattggctcatcgttttgagactgagattaggaacatattgaataatatctacaacaataataaagattataattggagaaaagaaaatctgtatgcaacctcccttgaattcagactacttagacaacatggctatcctgtttctcaagaggttttcaatggttttaaagacgaccagggaggcttcatttgtgatgatttcaagggaatactgagcttgcatgaagcttcgtattacagcttagaaggagaaagcatcatggaggaggcctggcaatttactagtaaacatcttaaagaagtgatgatcagcaagaacatggaagaggatgtatttgtagcagaacaagcgaagcgtgcactggagctccctctgcattggaaagtgccaatgttagaggcaaggtggttcatacacatttatgagagaagagaggacaagaaccaccttttacttgagctcgctaagatggagtttaacactttgcaggcaatttaccaggaagaactaaaagaaatttcagggtggtggaaggatacaggtcttggagagaaattgagctttgcgaggaacaggttggtagcgtccttcttatggagcatggggatcgcgtttgagcctcaattcgcctactgcaggagagtgctcacaatctcgatagccctaattacagtgattgatgacatttatgatgtctatggaacattggatgaacttgagatattcactgatgctgttgagaggtgggacatcaattatgctttgaagcaccttccgggctatatgaaaatgtgttttcttgcgctttacaactttgttaatgaatttgcttattacgttctcaaacaacaggattttgatttgcttctgagcataaaaaatgcatggcttggcttaatacaagcctacttggtggaggcgaaatggtaccatagcaagtacacaccgaaactggaagaatacttggaaaatggattggtatcaataacgggccctttaattataacgatttcatatctttctggtacaaatccaatcattaagaaggaactggaatttctagaaagtaatccagatatagttcactggtcatccaagattttccgtctgcaagatgatttgggaacttcatcggacgagatacagagaggggatgttccgaaatcaatccagtgttacatgcatgaaactggtgcctcagaggaagttgctcgtcaacacatcaaggatatgatgagacagatgtggaagaaggtgaatgcatacacagccgataaagactctcccttgactggaacaactactgagttcctcttgaatcttgtgagaatgtcccattttatgtatctacatggagatgggcatggtgttcaaaaccaagagactatcgatgtcggttttacattgctttttcagcccattcccttggaggacaaacacatggctttcacagcatctcctggcaccaaaggctga

SEQ ID NO: 104 - DNA, 1671 bp, Artificial Sequence
  DNA having modified codons, which encodes limonene synthase gene derived from Citrus limon (opt_ClLMS)
atggaccgccgtagcgcgaattaccagcccagtatttgggatcatgactttctgcagagcctgaacagtaattacacggacgaagcgtataagcgccgtgccgaagagctgcgcggtaaagttaagattgcgatcaaagatgtgatcgaaccgctggaccagctggagctgattgataacctgcagcgcctgggcctggcccatcgttttgaaaccgagatccgcaacatcctgaacaacatctacaacaacaacaaggattacaactggcgcaaggaaaatctgtacgccacgagcctggagtttcgcctgctgcgtcagcacggttatcccgtgagtcaggaagtctttaacggctttaaggatgaccagggcggttttatctgcgatgactttaaaggtattctgagcctgcatgaagcgagctactatagtctggaaggcgagagtatcatggaagaggcctggcagtttaccagcaaacacctgaaggaagttatgattagtaagaacatggaagaggatgtttttgtggccgaacaggcgaagcgtgccctggagctgcctctgcattggaaagtgccgatgctggaagcccgctggtttattcatatctatgaacgccgtgaggacaagaaccacctgctgctggaactggcgaaaatggagtttaatacgctgcaggccatctaccaggaagagctgaaagaaattagcggttggtggaaggataccggcctgggcgagaaactgagttttgcgcgcaaccgtctggttgccagctttctgtggagtatgggcatcgcgtttgaaccccagtttgcctactgtcgccgtgtgctgacgattagcatcgcgctgattaccgtcatcgatgacatttacgacgtttatggtacgctggatgaactggagatctttaccgacgcggttgaacgctgggatattaattacgccctgaagcacctgcctggctatatgaaaatgtgctttctggcgctgtacaactttgtgaatgaatttgcctactatgtcctgaagcagcaggattttgacctgctgctgagcatcaaaaacgcctggctgggcctgattcaggcgtacctggtcgaagccaaatggtaccatagcaaatatacccctaagctggaagagtatctggaaaatggtctggttagcatcacgggccccctgattatcaccattagctacctgagtggcacgaaccctattatcaaaaaggaactggagtttctggaaagcaatccggacatcgtgcactggagcagtaagatttttcgtctgcaggatgacctgggtacgagcagtgacgaaatccagcgcggcgatgtccctaaaagcattcagtgttacatgcatgagacgggtgccagtgaagaggtggcccgtcagcacattaaggatatgatgcgccagatgtggaaaaaggtcaatgcgtatacggccgataaagacagcccgctgaccggcaccacgaccgaatttctgctgaacctggtgcgcatgagccattttatgtacctgcatggcgacggtcacggcgttcagaatcaggaaacgatcgatgtgggctttaccctgctgtttcagccgattcccctggaggataagcacatggcgtttacggccagcccgggtaccaaaggctaa

SEQ ID NO: 105 - DNA, 67 bp, Artificial Sequence
  primer for amplifying opt_PsLMS
gtgtggaatcgtgagcggataacaatttcacacaaggagactgccatgcagcgccgtcgcggcaatt

SEQ ID NO: 106 - DNA, 36 bp, Artificial Sequence
  primer for amplifying opt_PsLMS
gtctcctgtgtgaaattacagcgtcacgggttccag

SEQ ID NO: 107 - DNA, 75 bp, Artificial Sequence
  primer for amplifying tac promoter region
cgttgttgccattgctgcaccctgttgacaattaatcatcggctcgtataatgtgtggaatcgtgagcggataac

SEQ ID NO: 108 - DNA, 23 bp, Artificial Sequence
  primer for amplifying ispA*
tttcacacaggagactgccatgg

SEQ ID NO: 109 - DNA, 42 bp, Artificial Sequence
  primer for amplifying ispA*
atgacttggttgagtctatttgttgcgctggatgatgtaatc

SEQ ID NO: 110 - DNA, 67 bp, Artificial Sequence
  primer for amplifying opt_AgLMS
gtgtggaatcgtgagcggataacaatttcacacaaggagactgccatgcagcgtcgcatcgcggatc

SEQ ID NO: 111 - DNA, 34 bp, Artificial Sequence
  primer for amplifying opt_AgLMS
gtctcctgtgtgaaattacagcgccaggggttcc

SEQ ID NO: 112 - DNA, 72 bp, Artificial Sequence
  primer for amplifying opt_MsLMS
gtgtggaatcgtgagcggataacaatttcacacaaggagactgccatggaacgccgtagcggcaattataac

SEQ ID NO: 113 - DNA, 38 bp, Artificial Sequence
  primer for amplifying opt_MsLMS
gtctcctgtgtgaaattaggcaaacggttcaaacagcg

SEQ ID NO: 114 - DNA, 66 bp, Artificial Sequence
  primer for amplifying opt_CuLMS
gtgtggaatcgtgagcggataacaatttcacacaaggagactgccatggaccgccgtagcgccaac

SEQ ID NO: 115 - DNA, 35 bp, Artificial Sequence
  primer for amplifying opt_CuLMS
gtctcctgtgtgaaattagcccttcgtacccgggc

SEQ ID NO: 116 - DNA, 69 bp, Artificial Sequence
  primer for amplifying opt_ClLMS
gtgtggaatcgtgagcggataacaatttcacacaaggagactgccatggaccgccgtagcgcgaattac

SEQ ID NO: 117 - DNA, 35 bp, Artificial Sequence
  primer for amplifying opt_ClLMS
gtctcctgtgtgaaattagcctttggtacccgggc